SEQUENCE LISTING (4 sequences)

SEQ ID NO: 1 - DNA, 21 bp, Artificial sequence
  Synthetic polynucleotide
  misc_feature: PD-L1-F primer
tggcatttgctgaacgcattt

SEQ ID NO: 2 - DNA, 22 bp, Artificial sequence
  Synthetic polynucleotide
  misc_feature: PD-L1-R primer
tgcagccaggtctaattgtttt

SEQ ID NO: 3 - DNA, 20 bp, Artificial sequence
  Synthetic polynucleotide
  misc_feature: Primer-PD-L1 promoter-F
tcagtcaccttgaagaggct

SEQ ID NO: 4 - DNA, 20 bp, Artificial sequence
  Synthetic polynucleotide
  misc_feature: Primer-PD-L1 promoter-R
tttcaccgggaagagtttcg

(19) United States
(12) Patent Application Publication
(10) Pub. No.: US 2022/0249564 A1
(43) Pub. Date: Aug. 11, 2022

(54) PDL1 POSITIVE NK CELL CANCER TREATMENT

(71) Applicant: City of Hope, Duarte, CA (US)

(72) Inventors: Jianhua Yu, Duarte, CA (US); Michael A. Caligiuri, Duarte, CA (US); Wenjuan Dong, Duarte, CA (US)

(73) Assignee: City of Hope, Duarte, CA (US)

(21) Appl. No.: 17/621,909

(22) PCT Filed: Jun. 24, 2020
(86) PCT No.: PCT/US20/39449

§ 371 Date: Dec. 22, 2021

Related U.S. Application Data
(60) Provisional application No. 62/866,511, filed on Jun. 25, 2019.

(51) Int. Cl. A61K 35/17; G01N 33/50; A61P 35/02; C12N 5/0783; A61K 45/06; A61K 39/395; C07K 16/28

(52) U.S. Cl. CPC A61K 35/17; A61K 2039/505; A61P 35/02; C12N 5/0646; A61K 45/06; A61K 39/39558; C07K 16/2827; C07K 2317/76; C12N 2502/30; C12N 2501/2312; C12N 2501/2315; C12N 2501/2318; C07K 2317/24; G01N 2333/70532; G01N 33/5047

(57) ABSTRACT

Provided herein are methods of treating cancer in a subject including detecting an amount of PD-L1(+) natural killer (NK) cells in a biological sample from the subject and treating the subject with an anti cancer therapy. Provided herein are methods of treating cancer in a patient including isolating natural killer (NK) cells from a subject, producing a population of PD-L1(+) NK cell from the isolated NK cells, and administering the population of PD-L1(+) NK cells into the patient.

### CROSS-REFERENCED APPLICATION

This application claims priority benefit to U.S. Ser. No. 62/866,511 filed Jun. 25, 2019, and is incorporated herein in its entirety.

### STATEMENT AS TO RIGHTS TO INVENTIONS MADE UNDER FEDERALLY SPONSORED RESEARCH AND DEVELOPMENT

This invention was made with government support under grant nos. CA210087, AI129582, and NS106170 awarded by the National Institutes of Health. The government has certain rights in the invention.

### BACKGROUND

Inhibition of the programmed death-1/programmed death ligand-1 (PD-1/PD-L1) pathway has become a very powerful therapeutic strategy for patients with cancer, and has shown unprecedented clinical responses in advanced liquid and solid tumors. Two PD-1 monoclonal antibodies (mAbs), pembrolizumab (Keytruda) and nivolumab (Opdivo) are FDA-approved to treat melanoma, kidney cancer, head and neck cancers, and Hodgkin's lymphoma. Three PD-L1 mAbs, atezolizumab (Tecentriq), avelumab (Bavencio), and durvalumab (Imfinzi), are FDA-approved to treat non-small cell lung cancer (NSCLC), bladder cancer, and Merkel cell carcinoma of the skin. However, the overall response rate to anti-PD-L1 therapy is still very low in patients with melanoma (26%), NSCLC (21%), and renal cell carcinoma (13%). In addition, anti-PD-L1 therapy can also show an unexplained clinical response in the absence of PD-L1 expression on the tumor cells.

Tumor cells in the tumor microenvironment (TME) can upregulate PD-L1 after encountering activated T cells via their secretion of interferon gamma (IFN-γ). Upon binding to PD-1, PD-L1 delivers a suppressive signal to T cells and an anti-apoptotic signal to tumor cells, leading to T cell dysfunction and tumor survival. Therefore, anti-PD-1/PD-L1 therapy aims to remove this immune suppression and activate the T cell response against cancer. It has been reported that PD-L1 is not only expressed on tumor cells but is also found on immune cells such as T cells, natural killer (NK) cells and macrophages within the TME. However, the function and the mechanism of action of PD-L1 on NK cells remain unexplored. It is also unknown as to whether and how anti-PD-L1 mAbs can modulate the function of these NK cells expressing PD-L1. Unraveling these mechanisms will likely play an important role in the clinical effectiveness of anti-PD-1/PD-L1 therapy.

NK cells comprise a group of innate cytolytic effector cells that participate in immune surveillance against cancer and viral infection. NK cells become cytolytic without prior activation especially when they encounter cells lacking self-MHC Class I molecules. Downregulation of MHC occurs in the setting of cancer, allowing NK cells to recognize and lyse malignant cells. Activated NK cells exert strong cytotoxic effects via multiple mechanisms involving perforin, granzyme B, TRAIL, or FASL. NK cells also produce IFN-γ, which not only directly affects target cells, but also activates macrophages and T cells to kill tumor cells or enhance the antitumor activity of other immune cell. However, the function of PD-L1 on NK cells and the underlying mechanisms in the normal or disease setting, as well as the involvement of PD-L1⁺ NK cells in anti-PD-L1 therapy has not been explored. (See for example 1-18)

Provided herein are, inter alia, solutions to these and other problems in the art.

### BRIEF SUMMARY

In an aspect, provided herein are methods of treating cancer in a subject including detecting an amount of PD-L1(+) natural killer (NK) cells in a biological sample from the subject and treating the subject with an anticancer therapy.

In an aspect, provided herein are methods of treating cancer in a patient including isolating natural killer (NK) cells from a subject thereby producing a population of isolated NK cells, deriving a population of PD-L1(+) NK cell from the population of isolated NK cells, and administering the population of PD-L1(+) NK cells into the patient.

In an aspect, provided herein are methods of treating cancer in a subject including administering an NK cell activating agent and an immunotherapeutic (e.g. an effective amount of an NK cell activating agent and an immunotherapeutic) to the subject.

### BRIEF DESCRIPTION OF THE DRAWINGS

FIGS. 1A-1G present data showing that PD-L1 expression was increased on natural killer (NK) cells following incubation with K562 myeloid leukemia cells in the presence of IL-2. FIG. 1A shows representative flow cytometry plots and summary data (n=17) showing PD-L1 expression on enriched healthy donor-derived NK cells incubated with or without K562 myeloid leukemia cells in the presence of IL-2 (10 ng/mL). FIG. 1B shows NK cells incubated with or without K562 myeloid leukemia cells in the presence of IL-2 (10 ng/mL), and relative PD-L1 mRNA expression measured by qRT-PCR. The experiment was repeated three times. FIG. 1C shows representative immunoblot and summary data (n=3) showing total PD-L1 protein in NK cells incubated with or without K562 myeloid leukemia cells in the presence of IL-2 (10 ng/mL). Total PD-L1 protein was measured by immunoblot and the relative expression rate was calculated by Image J. FIG. 1D shows immunofluorescence of unstimulated NK cells and sorted PD-L1⁺ NK cells following stimulation with K562 myeloid leukemia cells and then stained with PD-L1, CD56, and DAPI (nuclear stain). Images are shown at 10× magnification (scale bar, 5 μm) with the white square inset at further magnification. FIG. 1E shows NK cells were incubated with or without K562 myeloid leukemia cells in the presence of IL-2 (10 ng/mL), and secreted PD-L1 protein was measured by ELISA (n=6). FIG. 1F shows FACS-purified NK cells (purity >96%) isolated by fluorescence-activated cell sorting were incubated in the presence or absence of K562 cells. PD-L1 expression was measured by flow cytometry (n=5). FIG. 1G shows representative flow cytometry plots and summary data (n=4) showing the percentages of PD-L1⁺ NK cells from enriched NK cells incubated alone, incubated with K562 myeloid leukemia cells in transwell, or co-incubated directly with K562 myeloid leukemia cells in the presence of 10 ng/mL IL-2. Two paired groups were compared by paired t-test. One-way ANOVA with repeated measures or linear mixed model was used to compare 3 or more donor-matched groups. P values were adjusted by the Holm-Sidak method. NS=not significant.

FIGS. 2A-2M present data showing that PD-L1 expression on natural killer (NK) cells was associated with dynamic changes of NK cell function in a time dependent manner. FIG. 2A shows representative flow cytometry plots and summary data (n=5) showing the expression of CD107α and IFN-γ in PD-L1⁻ and PD-L1⁺ NK cells induced by co-incubation with K562 myeloid leukemia cells for 24 hours in the presence of 10 ng/mL IL-2. FIG. 2B shows PD-L1⁻ and PD-L1⁺ NK cells induced by co-incubation with K562 myeloid leukemia cells for 24 hours, were sorted to >96% purity from three (3) healthy donors and were then incubated with K562 myeloid leukemia cells at different effector/target ratios. Cytotoxicity was measured by ⁵¹Cr release assay. Giemsa staining (as shown in FIG. 2C, top panel) of PD-L1⁻ and PD-L1⁺ NK cells purified by fluorescence-activated cell sorting. Representative images are shown at 20× magnification (scale bar, 5 μm). Transmission electron microscopy images (as shown in FIG. 2C, bottom panel) of PD-L1⁻ and PD-L1⁺ NK cells. Left images: 17,000× magnification. Right image: 11,500× magnification (scale bar, 500 nm) with further magnification in the squared area. Arrows a and b: thickness of cytoplasm; Arrow c: liposome; Arrow d: mitochondria. FIG. 2D shows representative flow cytometry plots and summary data (n=4) of NK cells cultured with K562 myeloid leukemia cells for 72 hours in the presence of IL-2. Live and early apoptotic PD-L1⁻ and PD-L1⁺ NK cells were measured by Sytox blue and Annexin V staining. FIG. 2E shows PD-L1⁻ and PD-L1⁺ NK cells were sorted from three (3) healthy donors and cultured with feeder cells (feeder cells were K562 cells with membrane-bound IL-21, treated with 100 Gy radiation) for 20 days in the presence of IL-2. Cells were counted by trypan blue exclusion. Provided are representative flow cytometry plots and summary data (n=5) of cleaved-Caspase 3 (as shown in FIG. 2F) and Ki67 (as shown in FIG. 2G) in PD-L1⁻ and PD-L1⁺ NK cells. Percentages of PD-L1+NK cells in the peripheral blood of 48 healthy donors and 79 patients with AML at time of initial diagnosis (as shown in FIG. 2H). FIG. 2I shows PD-L1 expression on NK cells from healthy donors incubated with primary patient AML blasts (n=4). Percentages of PD-L1⁺ NK cells at time of diagnosis and at time of evaluation for response in AML patients who achieved a complete remission (CR) (as shown in FIG. 2J, n=paired groups of 31) and those who did not achieve a CR (as shown in FIG. 2K, n=paired groups of 16) following induction chemotherapy.

FIG. 2L shows percentages of PD-L1⁺ NK cells at time of evaluation for response in AML patients who did and did not achieved a CR following induction chemotherapy. FIG. 2M shows percentage change of PD-L1⁺ NK cells (calculated by comparing PD-L1⁺ NK cells at diagnosis and after treatment) in patients who achieve a CR and those who did not achieve a CR. Two paired groups were compared by paired t-test. One-way ANOVA with repeated measures or linear mixed model was used to compare three (3) or more donor-matched groups. P values were adjusted by the Holm-Sidak method. HD=healthy donor; AD=after diagnosis; CR=complete response; NCR=no complete response. MFI=mean fluorescence intensity.

FIGS. 3A-3D present data showing that anti-PD-L1 monoclonal antibody atezolizumab (AZ) activates PD-L1 signaling in NK cells and enhances NK cell function. FIG. 3A shows representative flow cytometry plots and summary data (n=5) showing the expression of CD107α and IFN-γ in fresh human NK cells from healthy donors stimulated with PD-L1 knock-out K562 cells for 20 hours and then treated with 20 μg/mL AZ for 4 hours. * P<0.05 by linear mixed model. The P values were corrected for multiple comparisons by Holm's method. FIG. 3B shows fresh human NK cells from healthy donors were transduced with empty vector (EV) or PD-L1 overexpression vector with or without AZ treatment prior to measure the expression of IFN-γ by flow cytometry. The experiment was repeated three times with three different donors. FIG. 3C shows NK cells enriched from healthy donors (n=6) were co-incubated with PD-L1 knock-out K562 cells for 20 hours and then treated with 20 μg/mL AZ at indicated time. NK cells were sorted and the relative levels of PD-L1 mRNA expression were measured by qRT-PCR. The experiment was repeated three times. FIG. 3D shows representative flow cytometry plots and summary data (n=5) demonstrating the expression of PD-L1 on NK cells increases in a time dependent manner after treatment with AZ. Two paired groups were compared by paired t-test. One-way ANOVA with repeated measures or linear mixed model was used to compare three (3) or more donor-matched groups. P values were adjusted by the Holm-Sidak method.

FIGS. 4A-4E present data showing that PD-L1 knockout (KO) mice and NK cell depletion show impaired anti-tumor activity in a YAC-1 tumor model with or without anti-PD-L1 mAb. Representative flow cytometry plots and summary data (n=5) of murine NK cell PD-L1 expression (as shown in FIG. 4A) and NK cell CD107α expression (as shown in FIG. 4B) in the spleen and lung of BALB/c mice after being challenged with PD-L1-knockout YAC-1 cells. Representative flow cytometry plots and summary data (n=5) of NK cell CD107α expression in the spleen (as shown in FIG. 4C) and lung (as shown in FIG. 4D) of wild-type (WT) and PD-L1^{-/-} BALB/c mice challenged with PD-L1-KO YAC-1 cells treated with or without anti-PD-L1 mAb. FIG. 4E shows number of PD-L1-KO YAC-1 cells in spleen in WT (wild type), NK cell depleted and PD-L1^{-/-} BALB/c mice (n=5) treated with or without anti-PD-L1 mAb. Two paired groups were compared by paired t-test. One-way ANOVA with repeated measures or linear mixed model was used to compare three (3) or more donor-matched groups. P values were adjusted by the Holm-Sidak method.

FIGS. 5A-5D present data showing the effects of the anti-PD-L1 mAb AZ and/or NK-activating cytokines on anti-tumor efficacy in vivo. FIG. 5A shows fresh human primary NK cells were injected intravenously (i.v.) into NOD scid gamma (NSG) mice without or with PD-L1-KO K562 myeloid leukemia cells followed by intraperitoneal (i.p.) injection of 1 μg IL-12 and 1 μg IL-15 per mouse every other day. After 6 days, mice were sacrificed and NK cells were isolated and assessed for PD-L1 expression by flow cytometry. Representative figures and summary data are shown (n=5). FIG. 5B shows fresh human primary NK cells and PD-L1-KO K562 myeloid leukemia cells were injected i.v. into NSG mice, followed by treatment with i.p. injection of AZ or PBS every other day. PBS instead of IgG1 was used as placebo because AZ lacks antibody-dependent cellular cytotoxicity activity. After 6 days (three treatments), mice were sacrificed and human NK cells were examined for their expression of granzyme B, CD107α, and IFN-γ, while the number of PD-L1-knockout K562 myeloid leukemia cells (as shown in FIG. 5C) was enumerated by flow cytometry. Representative figures and summary data (n=5) are shown. FIG. 5D shows survival curve of NSG mice intravenously (i.v.) injected with human primary NK cells and PD-L1-KO K562 myeloid leukemia cells followed by treatment with IL-2 plus IgG1, or IL-2 plus AZ, or IL-12, IL-15 and IL-18 plus IgG1, or IL-12, IL-15 and IL-18 plus AZ every other day for two weeks. Two paired groups were compared by paired t-test. One-way ANOVA with repeated measures or linear mixed model was used to compare three (3) or more donor-matched groups. P values were adjusted by the Holm-Sidak method. Kaplan-Meier method was used to estimate survival functions and log-rank test was applied to group comparisons.

FIGS. 6A-6J present data showing signaling pathways activating PD-L1⁺ NK cells. FIG. 6A shows gene expression profile using RNA microarray of PD-L1⁻ and PD-L1⁺ NK cells sorted from three healthy donors (D1, D2 and D3) following incubation with K562 myeloid leukemia cells as described in the Materials and Methods section. “D1+” represents PD-L1⁺ NK cells from donor 1, while “D1−” represents PD-L1⁻ NK cells from donor 1, each purified by FACS sorting. Similar definitions are applied to “D2+”, “D2−”, “D3+”, and “D3−”. Expression of Cd274 (PD-L1), Cd226, Tbx21, Eomes, Smad3 and Aka are highlighted by black arrows. FIG. 6B shows NK cells were incubated with or without K562 myeloid leukemia cells and in the presence or absence of AKT-pan inhibitor Afureserlib, PI3K-specific inhibitor wortmannin and P65-specific inhibitor TPCK at the concentration of 1 μM or 10 μM. The percentages of PD-L1+ NK cells were measured by flow cytometry. FIG. 6C shows inhibition rate of PD-L1⁺ NK cells was measured as the relative proportion of PD-L1⁺ cells in each treatment condition compared to untreated control (no inhibition). Data were generated from five (5) independent donors, and the experiment was repeated three times. FIG. 6D shows 293T cells were co-transfected with the PD-L1 promoter and the genes for each of the indicated proteins. Relative promoter activity was measured by luciferase assay after 48 hours. FIG. 6E shows the chromatin immunoprecipitation (ChIP) assay was employed to assess binding to the PD-L1 promoter with AKT (as shown in FIG. 6E) and p65 (as shown in FIG. 6F). The experiments were repeated three times. FIG. 6G shows expression of p-AKT and p-p38 were examined by immunoblot using β-actin as internal control. FIG. 6H shows PD-L1 expression on NK cells incubated with K562 cells and subsequent treatment with the anti-PD-L1 mAb AZ in the absence or presence of 1 μM of the p38 inhibitor SB202190 or 1 μM of the p38 inhibitor SB2035880 (n=4) as examined by flow cytometry. FIG. 6I shows the chromatin immunoprecipitation (ChIP) assay was employed to assess binding to the PD-L1 promoter with p38. The experiments were repeated three times.

FIG. 6J shows a representative example and summary data (n=5) quantifying the expression of CD107α and IFN-γ in NK cells following their incubation with K562 cells and subsequent treatment with the anti-PD-L1 mAb AZ in the absence or presence of 1 μM of the p38 inhibitor SB202190 or 1 μM of the p38 inhibitor SB2035880. Two paired groups were compared by paired t-test. One-way ANOVA with repeated measures or linear mixed model was used to compare three (3) or more donor-matched groups. P values were adjusted by the Holm-Sidak method.

FIGS. 7A-7F present data showing induction of PD-L1 expression on NK cells by K562 cells and/or PBMCs in the presence of IL-2. FIG. 7A shows representative flow cytometric plots illustrating the gating strategy used to gate on or to sort purified PD-L1⁺ NK cells by fluorescence-activated cell sorting (FACS) when primary human NK cells were incubated with carboxyfluorescein succinimidyl ester (CFSE)-labeled K562 myeloid leukemia cell. The induction of PD-L1 surface expression on the NK cells following 24 hour incubation with K562 cells is shown. FIG. 7B presents data showing NK cells were incubated with IL-2 (10 ng/ml, same for all panels) alone or with the supernatant taken from with K562 cells (Sup) or with the supernatant taken from K562 cells that had been incubated with NK cells in the presence of IL-2 (Co-Sup). PD-L1 surface density expression on NK cells cultured under these conditions was then compared with PD-L1 surface density expression on primary human NK cells incubated with K562 cells plus IL-2. PD-L1 expression was measured by flow cytometry. Representative FACS plot and summary data (n=4) are shown. FIG. 7C-G present data demonstrating PBMCs were incubated with K562 myeloid leukemia cells for 24 hour in the presence of IL-2, followed by assessment for PD-L1 surface density expression on various cells CD3⁻CD56⁺ NK cells (FIG. 7C), CD3⁺CD56⁺ NKT cells (FIG. 7D), CD3⁺CD8⁺ T cells (FIG. 7E), CD3⁺CD4⁺ T cells (FIG. 7F), and CD3⁻CD19⁺ B cells (FIG. 7G), as measured by flow cytometry (n=5). Two paired groups were compared by paired t-test. One-way ANOVA with repeated measures or linear mixed model was used to compare 3 or more donor-matched groups. P values were adjusted by the Holm-Sidak method. *, P<0.05; **, P<0.01; ****, P<0.0001; NS=not significant; FSC=forward scatter; SSC=side scatter; PBMCs=peripheral blood mononuclear cells; Sup=supernatant; Co-Sup=Co-culture supernatant.

FIGS. 8A-8F show the temporal relationship between NK cell activation and PD-L1 expression during incubation with K562 myeloid leukemia cells and the correlation between PD-L1+NK cells and treatment outcomes. FIG. 8A shows representative flow cytometry plots and summary data (n=4) showing the expression of CD107α, IFN-γ and PD-L1 in primary human NK cells when incubated with K562 myeloid leukemia cells at indicated time points in the presence of 10 ng/mL IL-2. FIG. 8B shows representative flow cytometry plots and summary data (n=4) of surface markers on primary NK cells isolated from healthy donors and incubated without or with K562 cells for 24 hours. FIGS. 8C-D demonstrate percentages of total NK cells at time of diagnosis and at time of evaluation for response following standard induction chemotherapy in AML patients who achieved a complete remission (CR) (n=paired groups of 31) as shown in FIG. 8C and those who did not achieve a CR (NCR; n=paired groups of 16 as shown in FIG. 8D. FIG. 8E shows the percentages of total NK cells at time of evaluation for response following standard induction chemotherapy in AML patients who did (CR) and did not achieve a CR (NCR). FIG. 8F shows percentage change of total NK cells (calculated by comparing total NK cells at diagnosis and at the time of evaluation for response following standard induction chemotherapy) in patients who achieve a CR and those who did not achieve a CR (NCR). Two paired groups were compared by paired t-test. One-way ANOVA with repeated measures was used to compare 3 or more donor-matched groups. P values were adjusted by Holm-Sidak method. Multiple comparison test is adjusted by the Holm-Sidak method. *, P<0.05; **, P<0.01; ***, P<0.001; ****, P<0.0001; NS, not significant.

FIGS. 9A-9B present data showing PD-L1 expression on NK cells induced by K562 cells and PD-L1 KO K562 cells. FIG. 9A shows histograms assessing PD-L1 expression on WT and PD-L1 KO K562 cells by flow cytometry, confirming that the PD-L1 KO K562 cells are negative for PD-L1 expression. The experiment was repeated three times. FIG. 9B shows data showing PD-L1 expression on NK cells incubated with K562 cells or PD-L1 KO K562 cells examined by flow cytometry with data summarized in the right panel (n=5). Two paired groups were compared by paired t-test. Linear mixed model was used to compare three (3) or more groups and P values were adjusted by the Holm's method. *, P<0.05; **, P<0.01; NS=not significant.

FIGS. 10A-10D present data showing the effects of PD-L1 on NK cells in PD-L1 KO YAC-1 tumor-bearing mice. FIG. 10A shows histograms and summary data showing flow cytometry of PD-L1 KO YAC-1 cells, confirming the cells were negative for PD-L1 expression. The experiment was repeated three times. FIG. 10B shows the number of PD-L1 KO YAC-1 tumor cells in the lungs of wild type (WT) and PD-L1^{-/-} mice treated without or with anti-PD-L1 mAb. Summary data are provided for n=5. FIG. 10C presents data showing the percentage of total NK cells was not significantly different in WT or PD-L1^{-/-} mice, each bearing PD-L1 KO YAC-1 tumors and each treated with either placebo or anti-PD-L1-mAb. Summary data are provided for n=5. FIG. 10D presents data showing the percentage of total NK cells following NK cell depletion was not significantly different in WT mice with each bearing PD-L1 KO YAC-1 tumors and each treated with either placebo or anti-PD-L1-mAb. Summary data are provided for n=5. Two paired groups were compared by paired t-test. One-way ANOVA with repeated measures or linear mixed model was used to compare three (3) or more donor-matched groups. P values were adjusted by the Holm-Sidak method. *, P<0.05; **, P<0.01; ***, P<0.001; ****, P<0.0001; NS=not significant.

FIGS. 11A-11F present data showing the induction of PD-L1 expression on NK cells by NK cell-activating cytokines. FIG. 11A shows flow cytometry plots and summary data (n=3) showing the percentage of PD-L1⁺ human NK cells under different conditions of cytokine stimulation (10 ng/mL for each cytokine) in the absence (top row) or presence (bottom row) of K562 myeloid leukemia cells. FIG. 11B shows flow cytometry plots and summary data (n=3) showing the expression of PD-L1 on human NK cells induced by IL-12 plus IL-18 (10 ng/mL for each cytokine) in a time dependent manner. FIGS. 11C-D show PD-L1+ and PD-L1⁻ NK cells were fractionated from bulk primary human NK cells (n=3) treated with IL-12 plus IL-18 (10 ng/mL for each) overnight were quantified for cytotoxicity as in shown in FIG. 11C using 4-hr standard ⁵¹Cr release assay. The cytotoxicity levels of total NK cells incubated with medium alone were served as control. The cytokine-treated bulk NK cells were permeabilized and gated PD-L1+ and PD-L1⁻ cells to measure the MFI of IFN-γ by flow cytometry assay as shown in FIG. 11D. FIG. 11E shows PD-L1 expression on NK cells treated with IL-12 plus IL-18 (10 ng/mL for each cytokine) with or without IFN-γ receptor 1 neutralizing mAb (αIFN-γR1 Nab) at 10 μg/mL or IFN-γ receptor 2 neutralizing mAb (αIFN-γR2 Nab) at 10 μg/mL or in combination of αIFN-γR1 Nab and αIFN-γR2 Nab at 10 μg/mL each. Summary graph (n=3) is shown at the bottom. FIG. 11F shows PD-L1 expression on NK cells induced by IFN-γ or IFN-γ in combination with indicated cytokines at 10 ng/mL for 24 hour. Summary graph (n=3) is shown at the bottom. Two-sample t test was used for 2-group comparisons. One-way ANOVA was used to compare three (3) or more groups and P values were adjusted by the Holm-Sidak method. **, P<0.01; ***, P<0.001; ****, P<0.0001; NS=not significant.

FIGS. 12A-12D present data showing that PD-L1 expression was associated with the susceptibility of target cells to NK cell lysis. FIG. 12A shows the expression of MHC Class I (HLA-A, B, C) molecules on various human leukemic cell lines as examined by flow cytometry. The experiment was repeated three times and summarized in graphical form to the right. FIGS. 12B-C demonstrate human NK cells isolated from healthy donors were incubated with indicated cell lines in the presence of 10 ng/mL IL-2 for 24 hour and assessed for NK cell expression of CD107α (n=4) as shown in FIG. 12B and PD-L1 (n=6) as shown in FIG. 12C as measured by flow cytometry and summarized in graphical form to the right. FIG. 12D presents data showing primary human NK cells were incubated with MV-4-11 human myeloid leukemia cells for the indicated time periods ranging from 24 hours to 96 hours, while the same NK cells were incubated with K562 myeloid leukemia cells for 24 hours. Each culture of NK cells had their expression of PD-L1 quantified by flow cytometry, and the data are summarized (n=4) in graphical form to the right. One-way ANOVA was used to compare three (3) or more groups and P values were adjusted by the Holm-Sidak method. *, P<0.05; **, P<0.01; ****, P<0.0001; NS=not significant.

FIG. 13 presents data showing induction of p38-NF-κB signaling in primary human NK cells by the anti-PD-L1 antibody AZ. FIG. 13 shows quantification of downstream kinase phosphorylation following NK cell activation when incubated with K562 myeloid leukemia cells without or with anti-PD-L1 mAb in the presence of IL-2 (10 ng/mL). The histograms provided in the upper panel represent quantification of various phosphorylated kinases expressed in NK cells, followed by a graphical summary of the data (n=3) below each histogram. One-way ANOVA with repeated measures or linear mixed model was used to compare three (3) or more donor-matched groups and P values were adjusted by the Holm-Sidak method. ****, P<0.0001; NS=not significant.

FIGS. 14A-B present flow cytometry data showing NK cells enriched using EasySep™ Human NK Cell Enrichment Kit (stemcell) from PBMC were treated with 10 ng/ml IL-12 and IL-18 for 16 hours, to induce PD-L1 expression. These NK cells were then incubated with Naïve T cells, activated T cells (by stimulation of CD3/CD28 beads) and activated T cells in the presence of 20 μg/ml atezolizumab (AZ) at 1:1 ratio for 72 hours. FIG. 14A shows the percentage of CD8⁺ T cells as examined by flow cytometry. FIG. 14B shows the apoptosis of CD8⁺ T cells as examined by flow cytometry (SYTOX™ staining shows dead cells). For FIGS. 14A-B, P values of multiple comparison were analyzed using one-way ANOVA. Multiple comparison test was adjusted by Holm-Sidak method. **, P<0.01; ***, P<0.001; ****, P<0.0001.

FIG. 15 presents flow cytometry data showing that expanded human primary NK cells express PD-L1 and the expression of PD-L1 can be further enhanced by anti-PD-L1 mAb (AZ). The human primary NK cells were expanded by adding the K562 feeder cells (feeder cells were K562 cells with membrane-bound IL21 (62), treated with 100 Gy radiation) in the presence of 10 ng/ml IL-2. Expanded human primary NK cells for 7 days with indicated medium (R10: RPMI1640+10% FBS; MACS: MACS medium+5% human serum; SCGM: SCGM medium+5% human serum) were treated with or without 5 ng/ml IL-12 and IL-18 for 20 h in the presence of AZ or not. The expression of PD-L1 were examined by flow cytometry. P values of multiple comparison were analyzed using one-way ANOVA. Multiple comparison test was adjusted by Holm-Sidak method. ****, P<0.0001.

FIG. 16 presents flow cytometry data showing that NK cells expressed PD-L1 in lung cancer patients. PBMC from lung cancer patients were isolated and examined for PD-L1 expression by flow cytometry. P values of paired groups were compared by paired t-test. *, P<0.05.

FIG. 17 shows a schematic illustration of NK cell activation via both encounter with a NK cell-susceptible tumor target such as the K562 myeloid leukemia cell line or an-anti-PD-L1 mAb binding to PD-L1. The K562 myeloid leukemia tumor cells activate NK cells via the PI3K/AKT signaling pathway, which activates NK-κB. NK-κB binds to the PD-L1 promoter and induces the expression of PD-L1. The binding of anti-PD-L1 mAb to PD-L1 activates p38, which further activates NK-κB to also induce the expression of PD-L1, in which the presence of excess anti-PD-L1 mAb forms a positive feedback-signaling loop.

### DETAILED DESCRIPTION

### I. Definitions

Before the present invention is further described, it is to be understood that this invention is not strictly limited to particular embodiments described, and as such may of course vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

As used herein, the term “cancer” is used in accordance with its plain ordinary meaning and refers to all types of cancer, neoplasm or malignant tumors found in mammals (e.g. humans), including leukemias, lymphomas, carcinomas and sarcomas. Examples of cancers that may be treated with a compound, composition, or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is lung cancer. In embodiments, the cancer is leukemia.

The term “leukemia” is used in accordance with its plain ordinary meaning and refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Examples of leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia. In embodiments, the cancer is acute myeloid leukemia.

The term “patient” or “subject in need thereof” is used in accordance with its plain ordinary meaning and refers to a living organism suffering from or prone to a disease or condition that can be treated by administration of a composition, compound, or method as provided herein. Non-limiting examples include humans, other mammals, bovines, rats, mice, dogs, monkeys, goat, sheep, cows, deer, and other non-mammalian animals. In some embodiments, a patient is human. In embodiments, the subject has, had, or is suspected of having cancer.

As used herein, the terms “control” or “control experiment” are used in accordance with its plain ordinary meaning and refer to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

As used herein, the terms “treating” or “treatment” are used in accordance with its plain ordinary meaning and refer to to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating includes preventing. In embodiments, treating does not include preventing.

As used herein, the term “prevent” is used in accordance with its plain ordinary meaning and refers to a decrease in the occurrence of disease symptoms in a patient. The prevention may be complete (no detectable symptoms) or partial, such that fewer symptoms are observed than would likely occur absent treatment.

By “an effective amount,” “a therapeutically effective amount.” “therapeutically effective dose or amount” and the like is intended an amount of cells, agents, or compounds described herein that brings about a positive therapeutic response in a subject in need of, such as an amount that restores function and/or results in the elimination and/or reduction of tumor and/or cancer cells. The exact amount (of cells or agents) required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein. A “combined therapeutically effective amount” or “combined therapeutically effective dose or amount dose” refers a combination of therapies that together brings about a positive therapeutic response in a subject in need of, such as an amount that restores function and/or results in the elimination and/or reduction of tumor and/or cancer cells.

As used herein, the term “immune response” is used in accordance with its plain ordinary meaning and refers to a response by an organism that protects against disease. The response can be mounted by the innate immune system or by the adaptive immune system, as well known in the art.

As used herein, the terms “natural killer cells” and “INK cells” are used in accordance with their plain ordinary meaning and refer to a type of cytotoxic lymphocyte involved in the innate immune system. The role NK cells play is typically analogous to that of cytotoxic T cells in the vertebrate adaptive immune response. NK cells may provide rapid responses to virus-infected cells, acting at around 3 days after infection, and respond to tumor formation. Typically, immune cells detect major histocompatibility complex (MHC) presented on infected cell surfaces, triggering cytokine release, causing lysis or apoptosis. NK cells typically have the ability to recognize stressed cells in the absence of antibodies and MHC, allowing for a much faster immune reaction.

As used herein, the term “PD-L1(+) natural killer (NK) cells” are natural killer cells that express PD-L1 protein.

As used herein, the term “T cells” or “T lymphocytes” are used in accordance with their plain ordinary meaning and refer to a type of lymphocyte (a subtype of white blood cell) involved in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

As used herein, the terms “tumor microenvironment”, “TME”, and “cancer microenvironment” are used in accordance with its plain ordinary meaning and refer to the non-neoplastic cellular environment of a tumor, including blood vessels, immune cells, fibroblasts, cytokines, chemokines, non-cancerous cells present in the tumor, and proteins produced

As defined herein, the terms “activation”, “activate”, “activating”, “activator” and the like are used in accordance with its plain ordinary meaning and refer to an interaction that positively affects (e.g. increasing) the activity or function of a protein or cell relative to the activity or function of the protein or cell in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein that is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

As used herein, the terms “agonist,” “activator,” “upregulator,” etc. are used in accordance with its plain ordinary meaning and refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As used herein, the terms “inhibition”, “inhibit”, “inhibiting” and the like are used in accordance with its plain ordinary meaning and refer to an interaction that negatively affecting (e.g. decreasing) the activity or function of the protein or cell relative to the activity or function of the protein or cell in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein or cell from an indirect interaction (e.g. an inhibitor binds to a protein that activates the target protein, thereby preventing target protein activation or cell activations).

As used herein, the terms “inhibitor,” “repressor” or “antagonist” or “downregulator” are used in accordance with its plain ordinary meaning and refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

As used herein, the term “expression” is used in accordance with its plain ordinary meaning and refers to any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

As used herein, the term “signaling pathway” is used in accordance with its plain ordinary meaning and refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

As used herein, the term “cytokine” is used in accordance with its plain ordinary meaning and refers to a broad category of small proteins (˜5-20 kDa) that are important in cell signaling. Cytokines are peptides, and cannot cross the lipid bilayer of cells to enter the cytoplasm. Cytokines are involved in autocrine signaling, paracrine signaling and endocrine signaling as immunomodulating agents. Cytokines

include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

As used herein, the terms “IFN-γ” and “interferon gamma” are used herein according to its plain and ordinary meaning and refer to a dimerized soluble cytokine that is the only member of the type II class of interferons. It plays a role in innate and adaptive immunity against viral, some bacterial and protozoal infections. IFNγ is an important activator of macrophages and inducer of Class II major histocompatibility complex (MEW) molecule expression. The importance of IFNγ in the immune system stems in part from its ability to inhibit viral replication directly and from its immunostimulatory and immunomodulatory effects. IFNγ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops.

As used herein, the terms “CD107α”, “CD107-alpha” “lysosomal-associated membrane protein 1”, “LAMP-1” and “lysosome-associated glycoprotein 1” are used in accordance with their plain ordinary meaning and refer to a glycoprotein from a family of lysosome-associated membrane glycoproteins. CD107α is a type I transmembrane protein which is expressed at high or medium levels in at least 76 different normal tissue cell types. It resides primarily across lysosomal membranes, and functions to provide selectins with carbohydrate ligands. CD107α has also been shown to be a marker of degranulation on lymphocytes such as CD8+ and NK cells.

As used herein, the terms “IL-12”, “IL12”, and “interleukin-12” are used in accordance with their plain ordinary meaning and refer to an interleukin that is naturally produced by dendritic cells, macrophages, neutrophils, and human B-lymphoblastoid cells in response to antigenic stimulation plays an important role in the activities of natural killer cells and T lymphocytes. IL-12 mediates enhancement of the cytotoxic activity of NK cells and CD8+ cytotoxic T lymphocytes. There may be a link between IL-2 and the signal transduction of IL-12 in NK cells. IL-2 stimulates the expression of two IL-12 receptors, IL-12R-β1 and IL-12R-β2, maintaining the expression of a critical protein involved in IL-12 signaling in NK cells. Enhanced functional response is demonstrated by IFN-γ production and killing of target cells.

As used herein, the terms “IL-15”, “interleukin-15” and “IL15” are used in accordance with their plain ordinary meaning and refer to a cytokine with structural similarity to interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells. As a pleiotropic cytokine, it plays an important role in innate and adaptive immunity.

As used herein, the terms “IL-18”, “interleukin-18”, “IL18”, “interferon-gamma inducing factor” are used in accordance with their plain ordinary meaning and refer to a proinflammatory cytokine that belongs to the IL-1 superfamily and is produced by macrophages and other cells. IL-18 works by binding to the interleukin-18 receptor, and together with IL-12, it induces cell-mediated immunity following infection with microbial products like lipopolysaccharide (LPS). After stimulation with IL-18, natural killer (NK) cells and certain T cells release another important cytokine called interferon-γ (IFN-γ) or type II interferon that plays an important role in activating the macrophages or other cells.

As used herein, the term “immunotherapy,” “immunotherapeutic” and “immunotherapeutic agent” are used in accordance with their plain ordinary meaning and refer to the treatment of disease by activating or suppressing the immune system. Immunotherapies designed to elicit or amplify an immune response are classified as activation immunotherapies, while immunotherapies that reduce or suppress are classified as suppression immunotherapies. Such immunotherapeutic agents include antibodies and cell therapy.

As used herein, the term “checkpoint inhibitor” is used in accordance with its plain ordinary meaning and refers to a drug, often made of antibodies, that unleashes an immune system attack on cancer cells. An important part of the immune system is its ability to tell between normal cells in the body and those it sees as “foreign.” This lets the immune system attack the foreign cells while leaving the normal cells alone. To do this, it uses “checkpoints” which are molecules on certain immune cells that need to be activated (or inactivated) to start an immune response, Cancer cells sometimes find ways to use these checkpoints to avoid being attacked by the immune system. Drugs that target these checkpoints are known as checkpoint inhibitors.

As used herein, the term “PD-1” is used in accordance with its plain ordinary meaning and refers to a checkpoint protein on immune cells called T cells. It acts as a type of “off switch” that helps keep the T cells from attacking other cells in the body. It does this when it attaches to PD-L1, a protein on some normal (and cancer) cells. When PD-1 binds to PD-L1, it basically tells the T cell to leave the other cell alone. Some cancer cells have large amounts of PD-L1, which helps them evade immune attack.

As used herein, the term “PD-L1” or “programmed death-ligand 1 (PD-L1)” is a 40 kDa type 1 transmembrane protein that plays a role in suppressing the adaptive arm of immune system during particular events such as pregnancy, tissue allografts, autoimmune disease and other disease states. It appears that upregulation of PD-L1 may allow cancers to evade the host immune system.

As used herein, the term “feeder cell” or “feeders” are used in accordance with their plain ordinary meaning and refer to adherent growth-arrested, but viable and bioactive, cells. These cells may be used as a substratum to condition the medium on which other cells, particularly at low or clonal density, are grown. In embodiments, the cells of the feeder layer are irradiated or otherwise treated so that they will not proliferate.

As used herein, the terms “K562 cell” and“K562 cell line” are used in accordance with their plain ordinary meaning and refer to a human immortalised myelogenous leukemia cell line derived from a 53-year-old female chronic myelogenous leukemia patient in blast crisis. K562 cells are of the erythroleukemia type. The cells are non-adherent and rounded, are positive for the bcr:abl fusion gene, and bear some proteomic resemblance to both undifferentiated granulocytes and erythrocytes.

As used herein, the term “anticancer agent” and “anticancer therapy” are used in accordance with their plain ordinary meaning and refer to a molecule or composition (e.g. compound, peptide, protein, nucleic acid, drug, antagonist, inhibitor, modulator) or regimen used to treat cancer through destruction or inhibition of cancer cells or tissues. Anticancer therapy includes chemotherapy, radiation therapy, surgery, targeted therapy, immunotherapy, and cell therapy Anticancer agents and/or anticancer therapy may be selective for certain cancers or certain tissues. In some embodiments, an anti-cancer therapy is an immunotherapy. In embodiments, anticancer agent or therapy may include a checkpoint inhibitor (e.g. administration of an effective amount of a checkpoint inhibitor). In embodiments, the anti-cancer agent or therapy is a cell therapy.

In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenyl acetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin (including recombinant interleukin II, or r1L.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™) erlotinib (Tarceva™), cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pembrolizumab nivolumab, atezolizumab, avelumab, durvalumab or the like.

As used herein, the terms “cell therapy” and “cellular therapy” are used in accordance with their plain ordinary meaning and refer to therapy in which cellular material such as for example cells is injected, grafted or implanted into a patient. The cells may be living cells. In embodiments, the cells are NK cells expressing PD-L1 protein.

As used herein the terms, “HLA”, “HLA type”, “human leukocyte antigen system”, and “human leukocyte antigen complex” are used in accordance with their plain ordinary meaning and refer to a gene complex encoding the major histocompatibility complex (MHC) proteins in humans. These cell-surface proteins are responsible for the regulation of the immune system in humans.

### II. Methods of Use

In an aspect, provided herein are methods of treating cancer in a subject including detecting an amount of PD-L1(+) natural killer (NK) cells in a biological sample from the subject and treating the subject with an anticancer therapy. In embodiments, PD-L1+ natural killer cells are NK cells that express PD-L1 protein.

In embodiments, the cancer is a neoplasm or malignant tumor. In embodiments, the cancer is a leukemia, lymphoma, carcinoma or sarcoma. In embodiments, the cancer is brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, thyroid cancer, breast cancer, cervical cancer, head & neck cancer, liver cancer, kidney cancer, lung cancer, ovarian cancer, uterine cancer, Hodgkin's Disease, or Non-Hodgkin's Lymphoma. In embodiments, the lung cancer is lung adenocarcinoma, lung squamous cell carcinoma, or non-small cell lung carcinoma. In embodiments, the cancer is leukemia. In embodiments, the cancer or leukemia is acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia. In embodiments, the cancer is acute myeloid leukemia.

In embodiments, the cancer includes PDL1-negative tumor cells. In embodiments, the cancer includes PDL1-positive tumor cells.

In embodiments, the methods provided herein include detecting an amount of PD-L1(+) natural killer (NK) cells in a biological sample from a subject. In embodiments, methods of detecting include flow cytometry, fluorescence-activated cell sorting, antibody cell staining, immunohistochemistry (IHC), reverse transcriptase-quantitative polymerase chain reaction (RT-qPCR), immunofluorescent assay, and a combination thereof. In embodiments, the method of detecting is flow cytometry. In embodiments, the method of detecting is fluorescence-activated cell sorting. In embodiments, the method of detecting is antibody cell staining. In embodiments, the method of detecting is immunohistochemistry (IHC). In embodiments, the method of detecting is reverse transcriptase-quantitative polymerase chain reaction (RT-qPCR). In embodiments, the method of detecting is immunofluorescent assay. In embodiments, the method of detecting is a combination of flow cytometry, fluorescence-activated cell sorting, antibody cell staining, immunohistochemistry (IHC), reverse transcriptase-quantitative polymerase chain reaction (RT-qPCR), and/or immunofluorescent assay.

In embodiments, the amount of PD-L1(+) NK cells is about equal to or greater than the amount of PD-L1(−) NK cells. In embodiments, the amount of PD-L1(+) NK cells is about equal to the amount of PD-L1(−) NK cells. In embodiments, the amount of PD-L1(+) NK cells is greater than the amount of PD-L1(−) NK cells. In embodiments, the amount of PD-L1⁺ NK cells in the biological sample from the subject is compared to the amount of PD-L1(−) NK cells in the same sample. In embodiments, the amount of PD-L1⁺ NK cells in the biological sample from the subject is compared to a control. In embodiments, the control is an amount of PD-L1(+) NK cells (e.g. average amount) found in healthy patients, cancer patients or the general population. In embodiments, the control is an amount of PD-L1(+) NK cells (e.g. average amount) found in healthy patients. In embodiments, the control is an amount of PD-L1(+) NK cells (e.g. average amount) found in cancer patients. In embodiments, the control is an amount of PD-L1(+) NK cells (e.g. average amount) found in general population.

In embodiments, the amount of PD-L1(+) NK cells correlates with response to anti-cancer therapy in that higher amounts of PD-L1(+) NK cells in a subject correlates to a higher probably that the subject will respond to anti-cancer therapy (e.g experience a decrease in the number o cancer cells or tumor size). In embodiments, more PD-L1(+) NK cells than PD-L1(−) NK cells correlates with increased response to anti-cancer therapy. In embodiments, more PD-L1(+) NK cells than PD-L1(−) NK cells correlates with a better response to anti-cancer therapy.

In embodiments, the methods provided herein include administration of an anticancer therapy. In embodiments, the anticancer therapy is selected from chemotherapy, radiation therapy, surgery, targeted therapy, immunotherapy, and cell therapy. In embodiments, the anticancer therapy is chemotherapy. In embodiments, the anticancer therapy is radiation therapy. In embodiments, the anticancer therapy is surgery. In embodiments, the anticancer therapy is targeted therapy. In embodiments, the anticancer therapy is immunotherapy. In embodiments, the anticancer therapy is cell therapy.

In embodiments, immunotherapy includes a checkpoint inhibitor (e.g. administration of an effective amount of a checkpoint inhibitor to the subject). In embodiments, the checkpoint inhibitor is a PD-1 inhibitor (e.g. administration of an effective amount of a PD-1 inhibitor to the subject). In embodiments, the PD-1 inhibitor is selected from pembrolizumab and nivolumab (e.g. administration of an effective amount of pembrolizumab or nivolumab to the subject). In embodiments, the PD-1 inhibitor is pembrolizumab (e.g. administration of an effective amount of pembrolizumab to the subject). In embodiments, the PD-1 inhibitor is nivolumab (e.g. administration of an effective amount of nivolumab to the subject). In embodiments, the checkpoint inhibitor is a PD-L1 inhibitor (e.g. administration of an effective amount of a PD-L1 inhibitor to the subject). In embodiments, the PD-L1 inhibitor is selected from atezolizumab, avelumab, and durvalumab (e.g. administration of an effective amount of atezolizumab, avelumab or durvalumab to the subject). In embodiments, the PD-L1 inhibitor is atezolizumab (e.g. administration of an effective amount of atezolizumab to the subject). In embodiments, the PD-L1 inhibitor is avelumab (e.g. administration of an effective amount of avelumab to the subject). In embodiments, the PD-L1 inhibitor is durvalumab (e.g. administration of an effective amount of to the subject).

In embodiments, the cell therapy includes PD-L1(+) NK cells. In embodiments, cell therapy includes administering cells such as NK cells directly into a subject. In embodiments, the NK cells express PD-L1 (denoted PD-L1(+)). In embodiments, the PD-L1(+) NK cells are enriched or purified. In embodiments, the PD-L1(+) NK cells are enriched. In embodiments, the PD-L1(+) NK cells are purified. In embodiments, enrichment and/or purification is achieved by obtaining NK cells from a mixture. Methods for enrichment and/or purification include but are not limited to cell separation based on cell density, size, and/or affinity for antibody-coated beads. The methods include, for example, adherence, filtration, centrifugation, panning, MACS (magnetic-activated cell sorting), and FACS (fluorescence activated cell sorting). In embodiments, the cell therapy includes bulk NK cells. In embodiments, the bulk NK cells include PD-L1(+) NK cells.

In embodiments, the anticancer therapy includes a checkpoint inhibitor and cell therapy. In embodiments, the anticancer therapy includes PD-L1 inhibitor and PD-L1(+) NK cells. In embodiments, the PD-L1 inhibitor is selected from atezolizumab, avelumab, and durvalumab and the PD-L1(+) NK cells are enriched or purified. In embodiments, the PD-L1 inhibitor is atezolizumab and the PD-L1(+) NK cells are enriched. In embodiments, the PD-L1 inhibitor is atezolizumab and the PD-L1(+) NK cells are purified. In embodiments, the anticancer therapy includes a checkpoint inhibitor and a cell therapy including bulk NK cells that include PD-L1(+) NK cells. In embodiments, the anticancer therapy includes a PD-L1 inhibitor and a cell therapy including bulk NK cells that include PD-L1(+) NK cells. In embodiments, the anticancer therapy includes atezolizumab and bulk NK cells that include PD-L1(+) NK cells.

In embodiments, the anticancer therapy includes a checkpoint inhibitor and an NK cell activating agent (e.g. administration of an effective amount of a checkpoint inhibitor and an NK cell activating agent to the subject). In embodiments, the checkpoint inhibitor is a PD-1 inhibitor. In embodiments, the PD-1 inhibitor is selected from pembrolizumab and nivolumab. In embodiments, the PD-1 inhibitor is pembrolizumab. In embodiments, the PD-1 inhibitor is nivolumab. In embodiments, the checkpoint inhibitor is a PD-L1 inhibitor. In embodiments, the PD-L1 inhibitor is selected from atezolizumab, avelumab, and durvalumab. In embodiments, the PD-L1 inhibitor is atezolizumab. In embodiments, the NK cell-activating agent is a cytokine. In embodiments, the NK cell-activating agent is a cytokine selected from IL-2, IL-12, IL-15, IL-18, and a combination thereof. In embodiments, the cytokine is IL-2. In embodiments, the cytokine is IL-12. In embodiments, the cytokine is IL-15. In embodiments, the cytokine is IL-18. In embodiments, the cytokine is a combination of L-2, IL-12, IL-15, and/or IL-18. In embodiments, the anticancer therapy includes pembrolizumab and IL-2. In embodiments, the anticancer therapy includes pembrolizumab and IL-12. In embodiments, the anticancer therapy includes pembrolizumab and IL-15. In embodiments, the anticancer therapy includes pembrolizumab and IL-18. In embodiments, the anticancer therapy includes pembrolizumab and a combination of IL-2, IL-12, IL-15, and/or IL-18. In embodiments, the anticancer therapy includes nivolumab and IL-2. In embodiments, the anticancer therapy includes nivolumab and IL-12. In embodiments, the anticancer therapy includes nivolumab and IL-15. In embodiments, the anticancer therapy includes nivolumab and IL-18. In embodiments, the anticancer therapy includes nivolumab and a combination of IL-2, IL-12, IL-15, and/or IL-18. In embodiments, the anticancer therapy includes avelumab and IL-2. In embodiments, the anticancer therapy includes avelumab and IL-12. In embodiments, the anticancer therapy includes avelumab and IL-15. In embodiments, the anticancer therapy includes avelumab and IL-18. In embodiments, the anticancer therapy includes avelumab and a combination of IL-2, IL-12, IL-15, and/or IL-18. In embodiments, the anticancer therapy includes durvalumab and IL-2. In embodiments, the anticancer therapy includes durvalumab and IL-12. In embodiments, the anticancer therapy includes avelumab and IL-15. In embodiments, the anticancer therapy includes durvalumab and IL-18. In embodiments, the anticancer therapy includes durvalumab and a combination of IL-2, IL-12, IL-15, and/or IL-18. n embodiments, the anticancer therapy includes atezolizumab and IL-2. In embodiments, the anticancer therapy includes atezolizumab and IL-12. In embodiments, the anticancer therapy includes atezolizumab and IL-15. In embodiments, the anticancer therapy includes atezolizumab and IL-18. In embodiments, the anticancer therapy includes atezolizumab and a combination of IL-2, IL-12, IL-15, and/or IL-18.

In an aspect, provided herein are methods of treating cancer in a patient including isolating natural killer (NK) cells from a subject thereby producing a population of isolated NK cells, deriving a population of PD-L1(+) NK cell from the population of isolated NK cells, and administering the population of PD-L1(+) NK cells (e.g. an effective amount of PD-L1(+) NK cells) into the patient. In embodiments, methods of isolating natural killer cells include obtaining NK cells from a biological sample from a subject. Methods for isolating natural killer cells include but are not limited to cell separation based on cell density, size, and/or affinity for antibody-coated beads. The methods include, for example, adherence, filtration, centrifugation, panning, MACS (magnetic-activated cell sorting), and FACS (fluorescence activated cell sorting). In embodiments, deriving a population of PD-L1(+) NK cells from the isolated natural killer cells include isolating, enriching, and/or purifying PD-L1(+) cells. Such methods include but are not limited to cell separation based on cell density, size, and/or affinity for antibody-coated beads. The methods include, for example, adherence, filtration, centrifugation, panning, MACS (magnetic-activated cell sorting), and FACS (fluorescence activated cell sorting). In embodiments, the PD-L1(+) NK cells are administered to the patient. In embodiments, deriving a population of PD-L1(+) cells from a population of NK cells includes genetically engineering expression of PD-L1 in the NK cells. Such methods of genetic engineering are known and include recombinant protein expression in human cells. Specifically, NK cells may be transfected with an expression vector capable of expressing functional PD-L1, thereby producing PD-L1(+) NK cells.

In embodiments, the cancer is a cancer or tumor as described above.

In embodiments, the patient is selected from a patient diagnosed with cancer, a cancer patient relapsed from a treatment, or a cancer patient that has undergone hematopoietic stem cell transplantation. In embodiments, the patient is a patient diagnosed with cancer. In embodiments, the patient is a cancer patient relapsed from a treatment. In embodiments, the patient is a cancer patient that has undergone hematopoietic stem cell transplantation.

In embodiments, the patient has PD-L1 (+) NK cells, has no PD-L1 (+) NK cells, has an NK cell deficiency, or has NK cell suppression. In embodiments, the patient has PD-L1 (+) NK cells. In embodiments, the patient has no PD-L1 (+) NK cells. In embodiments, having no PD-L1 (+) NK cells includes having no detectable levels of PD-L1(+) NK cells. In embodiments, have no PD-L1 (+) NK cells includes having low levels of PD-L1(+) cells compared to a control. In embodiments, the control is a reference number of PD-L1(+) cells. In embodiments, the control is the average or mean number of PD-L1(+) cells in a healthy individual. In embodiments, the patient has an NK cell deficiency. In embodiments, the patient has NK cell suppression. In embodiments, NK cell suppression includes reduced NK cell activity, reduced NK cell number, and or reduced NK cell function.

In embodiments, the methods provided herein include isolating natural killer (NK) cells from a subject thereby producing a population of isolated NK cells. In embodiments, methods of isolating NK cells includes obtaining a population of cells from a subject where the population of cells includes NK cells. In embodiments, NK cells are isolated from the population of cells by any known method including but not limited to fluorescence-activated cell sorting, magnetic bead separation, and/or column purification. In embodiments, the method of isolating NK cells is fluorescence-activated cell sorting. In embodiments, the method of isolating NK cells is magnetic bead separation. In embodiments, the method of isolating NK cells is column purification. In embodiments, the method of isolating NK cells is a combination of fluorescence-activated cell sorting, magnetic bead separation, and/or column purification.

In embodiments, the methods include isolating NK cells from a subject. In embodiments, the subject is selected from an autologous cancer patient, a healthy donor, a matched heterologous hematopoietic stem cell donor, and a partially matched heterologous hematopoietic stem cell donor. In embodiments, the subject is an autologous cancer patient. The term “autologous cancer patient” refers to a cancer subject who is to be treated with methods of treating cancer described herein. In embodiments, the subject is a healthy donor. In embodiments, the healthy donor is a blood donor. In embodiments, the healthy donor is a PBMC (peripheral blood mononuclear cell) donor. In embodiments, the subject is a matched heterologous hematopoietic stem cell donor. The term “matched heterologous hematopoietic stem cell donor” refers to a subject from which NK cells are isolated has matching tissue type as the patient to be treated. Matching tissue type can be HLA type. In embodiments, the subject is a partially matched heterologous hematopoietic stem cell donor. The term “partially matched heterologous hematopoietic stem cell donor” refers to a subject from which NK cells are isolated has partially matching tissue type as the patient to be treated. Matching tissue type can be HLA type.

In embodiments, the methods provided herein include deriving a population of PD-L1(+) NK cells from the population of isolated NK cells. In embodiments, the method of deriving includes expanding PD-L1(+) NK cells by exposing the isolated NK cells to a feeder cell thereby producing a population of PD-L1(+) NK cell. In embodiments, the feeder cell is a K562 cell. In embodiments, the feeder cell is a K562 cell expressing IL-15 and IL-21.

In embodiments, the method of deriving a population of PD-L1(+) NK cells includes fluorescence-activated cell sorting, magnetic bead separation, and/or column purification thereby producing a population of PD-L1(+) NK cell. The methods include obtaining PD-L1(+) cells from a mixture of cells in a sample. The methods may be based on separation by cell density, size, and/or affinity for antibody-coated beads. The methods include, for example, adherence, filtration, centrifugation, panning, MACS (magnetic-activated cell sorting), and FACS (Fluorescence activated cell sorting. In embodiments, the method of deriving is fluorescence-activated cell sorting. In embodiments, the method of deriving is magnetic bead separation. In embodiments, the method of deriving is column purification.

In embodiments, the methods of deriving a population of PD-L1(+) NK cells include exposing the isolated NK cells to an NK activating agent to induce PD-L1 expression thereby producing a population of PD-L1(+) NK cell. In embodiments, the NK cell-activating agent is a feeder cell. In embodiments, exposing includes co-culturing isolated NK cells with a feeder cell. In embodiments, the feeder cell is a K562 cell. In embodiments, the feeder cell is a K562 cell expressing IL-15 and IL-21. In embodiments, exposing includes adding NK cell-activating an agent. In embodiments, the NK cell-activating agent is a cytokine. In embodiments, the NK cell-activating agent is a cytokine selected from IL-2, IL-12, IL-15, IL-18, and a combination thereof. In embodiments, the cytokine is IL-2. In embodiments, the cytokine is IL-12. In embodiments, the cytokine is IL-15. In embodiments, the cytokine is IL-18. In embodiments, the cytokine is a combination of L-2, IL-12, IL-15, and/or IL-18.

In embodiments, the population of PD-L1(+) NK cell is expanded prior to administering into the patient. Methods of expanding PD-L1(+) NK cells include exposing the PD-L1(+) NK cells to NK activating agents as described herein.

In embodiments, the methods of deriving a population of PD-L1(+) NK cells include genetically engineering PD-L1 expression in the population of isolated NK cells thereby producing a population of PD-L1(+) NK cell. Such methods of genetic engineering are known and include recombinant protein expression in human cells. Specifically, NK cells may be transfected with an expression vector capable of expressing functional PD-L1, thereby producing PD-L1(+) NK cells.

In embodiments, the methods provided herein further include administering an anticancer therapy (e.g. administration of an effective amount of an anticancer compound or chemotherapeutic agent to the subject). The anticancer therapy may include chemotherapy, radiation therapy, surgery, targeted therapy, immunotherapy, cell therapy, and/or a combination thereof. In embodiments, the methods provided herein further include administering chemotherapy (e.g. administration of an effective amount of the therapy to the subject). In embodiments, the methods provided herein further include administering radiation therapy. In embodiments, the methods provided herein further include administering surgery. In embodiments, the methods provided herein further include administering targeted therapy. In embodiments, the methods provided herein further include administering immunotherapy (e.g. administration of an effective amount of an immunotherapeutic agent to the subject). In embodiments, the methods provided herein further include administering cell therapy (e.g. administration of an effective amount of therapeutic cells to the subject). In embodiments, the methods provided herein further include administering a combination of chemotherapy, radiation therapy, surgery, targeted therapy, immunotherapy and cell therapy.

In embodiments, the immunotherapy includes administering an effective amount of a checkpoint inhibitor (e.g. administration of an effective amount of a checkpoint inhibitor to the subject). In embodiments, the checkpoint inhibitor is a PD-1 inhibitor (e.g. administration of an effective amount of a PD-1 inhibitor to the subject). In embodiments, the PD-1 inhibitor is selected from pembrolizumab and nivolumab (e.g. administration of an effective amount of pembrolizumab or nivolumab to the subject). In embodiments, the PD-1 inhibitor is pembrolizumab (e.g. administration of an effective amount of pembrolizuma to the subject). In embodiments, the PD-1 inhibitor is nivolumab (e.g. administration of an effective amount of nivolumab to the subject). In embodiments, the checkpoint inhibitor is a PD-L1 inhibitor (e.g. administration of an effective amount of a PD-L1 inhibitor to the subject). In embodiments, the PD-L1 inhibitor is selected from atezolizumab, avelumab, and durvalumab (e.g. administration of an effective amount of atezolizumab, avelumab, or durvalumab to the subject). In embodiments, the PD-L1 inhibitor is atezolizumab (e.g. administration of an effective amount of atezolizumab to the subject). In embodiments, the PD-L1 inhibitor is avelumab (e.g. administration of an effective amount of avelumab to the subject). In embodiments, the PD-L1 inhibitor is durvalumab (e.g. administration of an effective amount of to the subject).

In embodiments, the anticancer therapy includes administering an effective amount of an NK cell-activating agent. In embodiments, the NK cell-activating agent cytokine. In embodiments, the NK cell-activating agent is a cytokine selected from IL-2, IL-12, IL-15, IL-18, and a combination thereof. In embodiments, the cytokine is IL-2. In embodiments, the cytokine is IL-12. In embodiments, the cytokine is IL-15. In embodiments, the cytokine is IL-18. In embodiments, the cytokine is a combination of L-2, IL-12, IL-15, and/or IL-18.

In an aspect, provided herein are methods of treating cancer in a subject including administering an NK cell-activating agent and an immunotherapeutic agent in a combined effective amount to the subject.

In embodiments, the cancer is a cancer or tumor as described above.

In embodiments, the NK cell-activating agent is a cytokine. In embodiments, the NK cell-activating agent is a cytokine selected from IL-2, IL-12, IL-15, IL-18, and a combination thereof. In embodiments, the cytokine is IL-2. In embodiments, the cytokine is IL-12. In embodiments, the cytokine is IL-15. In embodiments, the cytokine is IL-18. In embodiments, the cytokine is a combination of L-2, IL-12, IL-15, and/or IL-18.

In embodiments, immunotherapy includes a checkpoint inhibitor (e.g. administration of an effective amount of a checkpoint inhibitor to the subject). In embodiments, the checkpoint inhibitor is a PD-1 inhibitor (e.g. administration of an effective amount of a PD-1 inhibitor to the subject). In embodiments, the PD-1 inhibitor is selected from pembrolizumab and nivolumab (e.g. administration of an effective amount of pembrolizumab or nivolumab to the subject). In embodiments, the PD-1 inhibitor is pembrolizumab (e.g. administration of an effective amount of pembrolizumab to the subject). In embodiments, the PD-1 inhibitor is nivolumab (e.g. administration of an effective amount of nivolumab to the subject). In embodiments, the checkpoint inhibitor is a PD-L1 inhibitor (e.g. administration of an effective amount of a PD-L1 inhibitor to the subject). In embodiments, the PD-L1 inhibitor is selected from atezolizumab, avelumab, and durvalumab (e.g. administration of an effective amount of atezolizumab, avelumab or durvalumab to the subject). In embodiments, the PD-L1 inhibitor is atezolizumab (e.g. administration of an effective amount of atezolizumab to the subject). In embodiments, the PD-L1 inhibitor is avelumab (e.g. administration of an effective amount of avelumab to the subject). In embodiments, the PD-L1 inhibitor is durvalumab (e.g. administration of an effective amount of to the subject).

In embodiments, the methods of treating cancer in a subject include administering an NK cell-activating cytokine and an immunotherapeutic agent in combined effective amount. In embodiments, methods of treating cancer in a subject include administering an NK cell-activating cytokine and a checkpoint inhibitor in a combined effective amount. In embodiments, methods of treating cancer in a subject include administering an NK cell-activating cytokine selected from IL-2, IL-12, IL-15, IL-18, and a combination thereof and a checkpoint inhibitor selected from a PD-1 inhibitor and a PD-L1 inhibitor in a combined effective amount.

In embodiments, methods of treating cancer in a subject including administering an NK cell-activating cytokine selected from IL-2, IL-12, IL-15, IL-18, and a combination thereof and a PD-1 inhibitor selected from pembrolizumab and nivolumab, in a combined effective amount. In embodiments, methods of treating cancer in a subject include administering IL-2, IL-12, IL-15, or IL-18 and pembrolizumab or nivolumab, in a combined effective amount. In embodiments, methods of treating cancer in a subject including administering IL-2 and pembrolizumab, in a combined effective amount. In embodiments, methods of treating cancer in a subject include administering IL-2 and nivolumab, in a combined effective amount. In embodiments, methods of treating cancer in a subject include administering IL-12 and pembrolizumab, in a combined effective amount. In embodiments, methods of treating cancer in a subject include administering IL-12 and nivolumab, in a combined effective amount. In embodiments, methods of treating cancer in a subject include administering IL-15 and pembrolizumab, in a combined effective amount. In embodiments, methods of treating cancer in a subject include administering IL-15 and nivolumab, in a combined effective amount. In embodiments, methods of treating cancer in a subject include administering IL-18 and pembrolizumab, in a combined effective amount. In embodiments, methods of treating cancer in a subject include administering IL-18 and nivolumab, in a combined effective amount. In embodiments, methods of treating cancer in a subject include administering a combination of IL-2, IL-12, IL-15, and/or IL-18 and pembrolizumab or nivolumab, in a combined effective amount.

In embodiments, methods of treating cancer in a subject include administering IL-2, IL-12, IL-15, or IL-18 and atezolizumab, avelumab, or durvalumab, in a combined effective amount. In embodiments, methods of treating cancer in a subject include administering IL-2 and atezolizumab, in a combined effective amount. In embodiments, methods of treating cancer in a subject include administering IL-2 and avelumab, in a combined effective amount. In embodiments, methods of treating cancer in a subject include administering IL-2 and durvalumab, in a combined effective amount. In embodiments, methods of treating cancer in a subject include administering IL-12 and atezolizumab, in a combined effective amount. In embodiments, methods of treating cancer in a subject include administering IL-12 and avelumab, in a combined effective amount. In embodiments, methods of treating cancer in a subject include administering IL-12 and durvalumab. In embodiments, methods of treating cancer in a subject include administering IL-15 and atezolizumab, in a combined effective amount. In embodiments, methods of treating cancer in a subject including administering IL-15 and avelumab, in a combined effective amount. In embodiments, methods of treating cancer in a subject include administering IL-15 and durvalumab, in a combined effective amount. In embodiments, methods of treating cancer in a subject include administering IL-18 and atezolizumab, in a combined effective amount. In embodiments, methods of treating cancer in a subject include administering IL-18 and avelumab, in a combined effective amount. In embodiments, methods of treating cancer in a subject include administering IL-18 and durvalumab, in a combined effective amount. In embodiments, methods of treating cancer in a subject include administering a combination of IL-2, IL-12, IL-15, and/or IL-18 and atezolizumab, avelumab, or durvalumab, in a combined effective amount.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: The Mechanism of Anti-PD-L1 Efficacy Against PD-L1 Negative Tumors Identifies NK Cells Expressing PD-L1 as a Cytolytic Effector

The experimental results provided herein demonstrate that some tumor cells can induce PD-L1 expression on natural killer (NK) cells via AKT signaling, resulting in enhanced NK cell function and preventing cell exhaustion. Anti-PD-L1 monoclonal antibody (mAb) directly acts on PD-L1⁺ NK cells against PD-L1⁻ tumors. Combination therapy of anti-PD-L1 mAb and NK cell activating cytokines significantly improved therapy efficacy in Non-SCID gamma (NSG) mice bearing human NK cells and PD-L1⁻ human leukemia when compared to monotherapy. The discovery of a PD-1-independent mechanism of anti-tumor efficacy via the activation of PD-L1⁺ NK cells with anti-PD-L1 mAb offers new insights into NK cell activation and may provide a potential explanation as to why patients lacking PD-L1 expression on tumor cells still respond to anti-PD-L1 mAb therapy.

Targeting PD-L1 to overcome T-cell exhaustion is successful to treat cancer, while patients lacking tumor PD-L1 expression can respond to anti-PD-L1 therapy. Data herein provides a possible explanation for this by characterizing PD-L1⁺ NK cells in acute myeloid leukemia (AML) patients and in animal models, which unraveled a PD-1 independent mechanism directly involving NK cells.

The experimental study herein demonstrated that some tumor cell lines and acute myeloid leukemia (AML) blasts from patients can upregulate PD-L1 on NK cells. PD-L1⁺ NK cells are activated effectors exerting enhanced cytotoxic activity against target cells in vitro compared to PD-L1⁻ NK cells. NK cells from a majority of AML patients expressed moderate to high levels PD-L1 and the change in its level of expression following chemotherapy correlated with clinical response. Further, in vivo, anti-PD-L1 mAb treatment in combination with NK cell-activating cytokines significantly enhanced NK cell antitumor activity against myeloid leukemia lacking PD-L1 expression, suggesting that anti-PD-L1 therapy may have a unique therapeutic role in select patients with AML who lack PD-L1 expression and are distinct from patients with PD-L1⁺ tumors. This mechanism of direct immune cell activation with anti-PD-L1 therapy that is PD-1-independent may explain the efficacy of the anti-PD-L1 checkpoint inhibitor in some tumors that lack PD-L1 expression.

Results

PD-L1 Expression on NK Cells after Encountering Tumor Cells

Expression of PD-L1 has been extensively reported on tumor cells and its binding to PD-1 on T cells suppresses the function of PD-1⁺ T cells [see, for example, 19]. The expression of PD-L1 on immune cells has also been reported on macrophages, T cells and NK cells [for example, 11-14]. However, the mechanism of induction and function of PD-L1 on NK cells remains unknown.

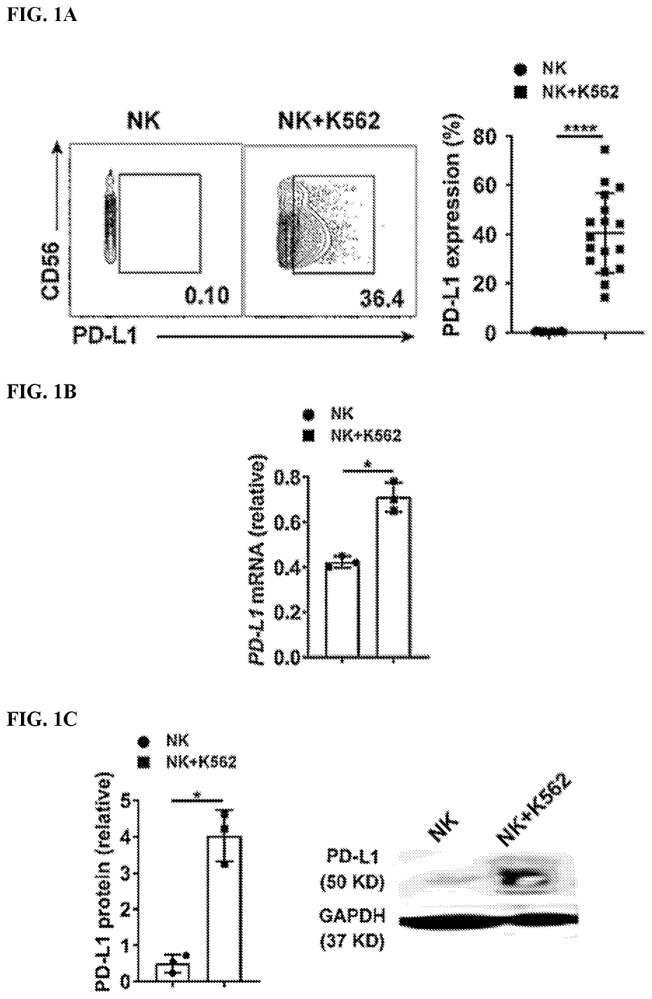

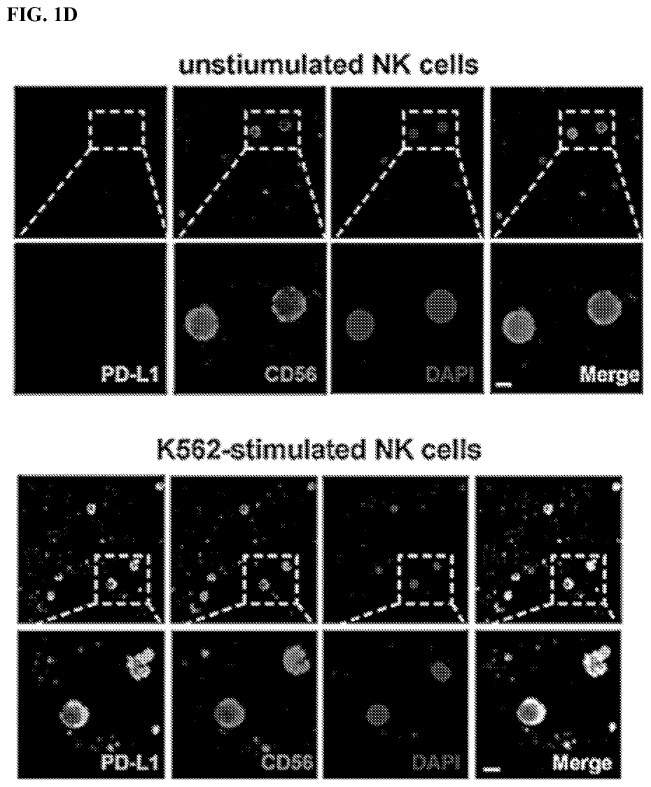

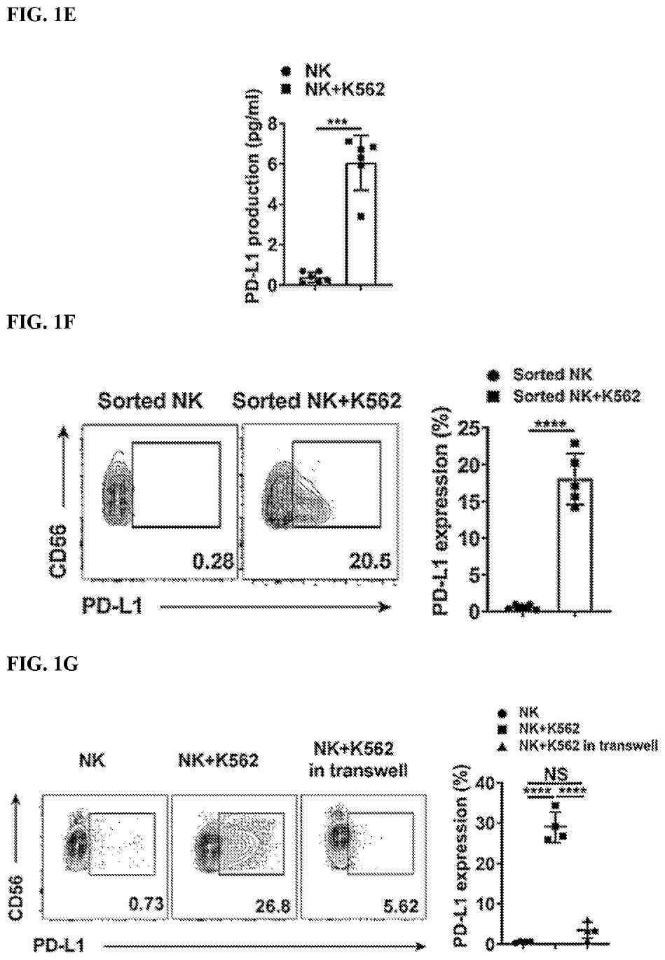

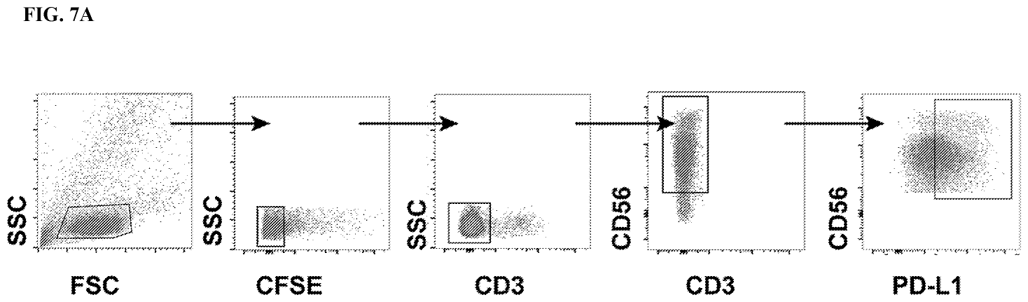

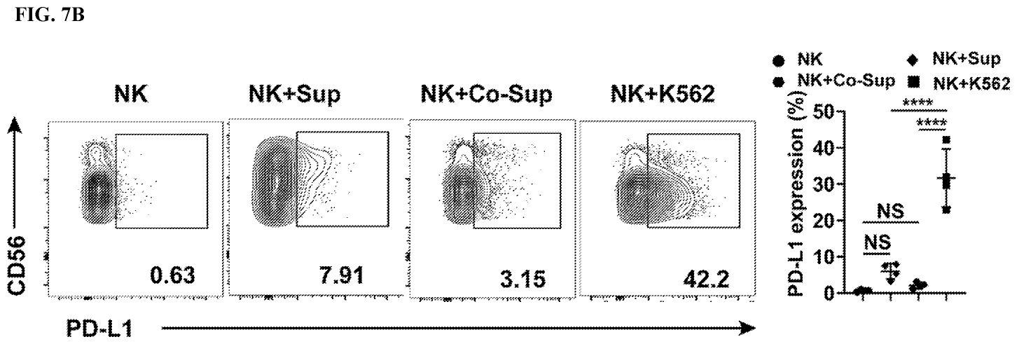

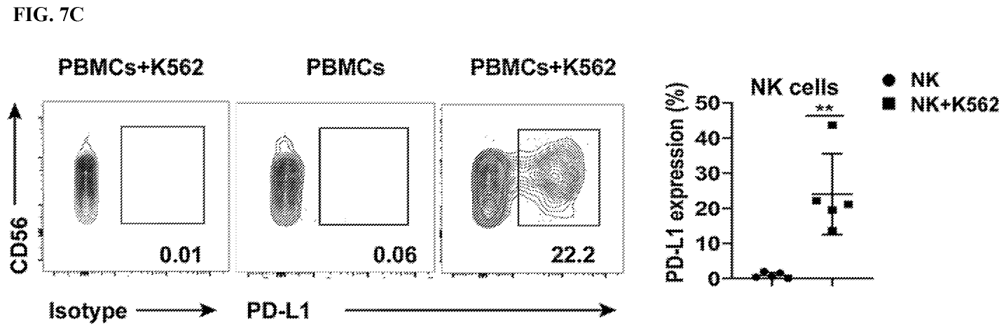

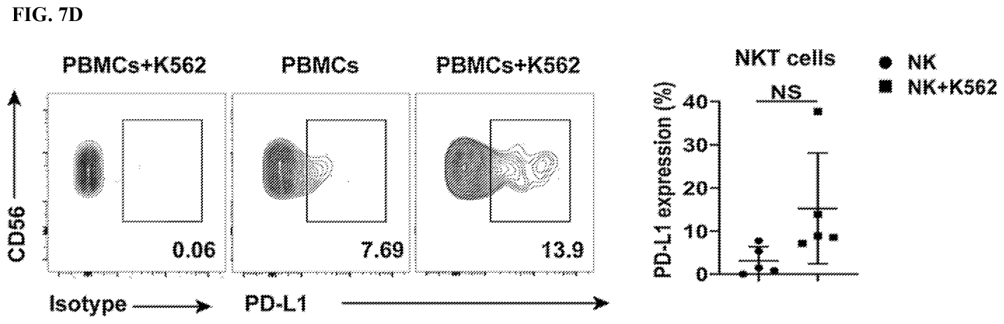

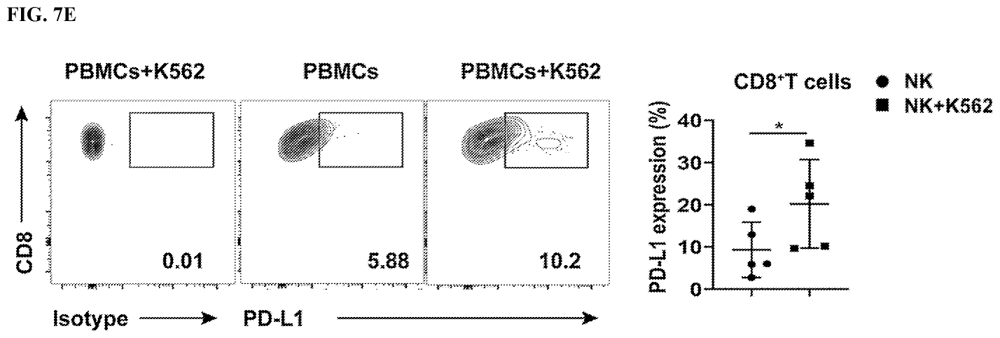

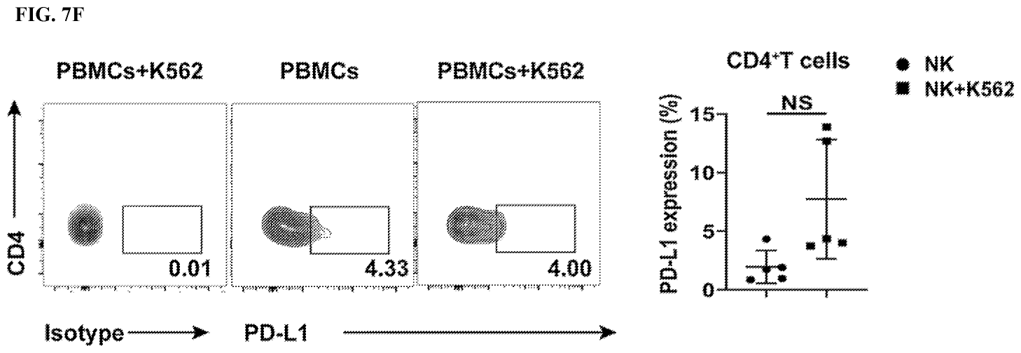

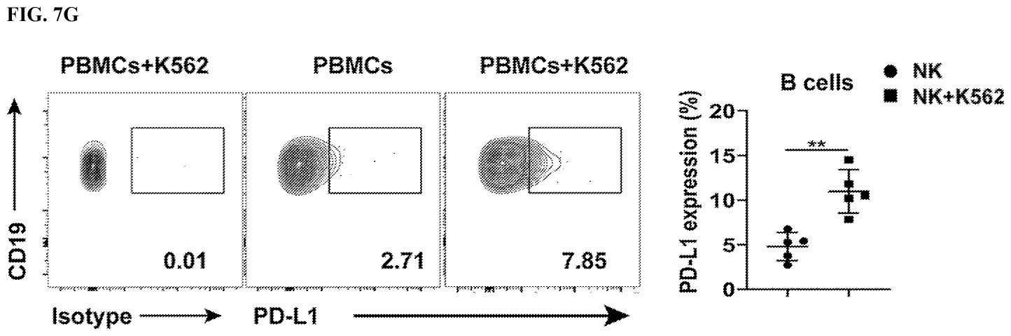

Experiments were conducted to enrich fresh human NK cells from healthy donors and co-cultured them with PD-L1^{lo/-} target tumor cells, the K562 myeloid leukemia cell line. Data demonstrated that anywhere from 14.2-74.4% of NK cells expressed PD-L1 after encountering K562 cells (FIG. 1A and FIG. 7A). The RNA and protein levels for PD-L1 were both markedly increased (FIGS. 1B and 1C). To confirm the expression of PD-L1 on NK cells, both PD-L1⁻ and PD-L1⁺ NK cells were stained with human NK cell surface marker CD56. Immunofluorescence images showed that PD-L1 localized with CD56 on PD-L1⁺ NK cells (FIG. 1D). In addition to its expression on the NK cell surface, PD-L1 can also be secreted (FIG. 1E). To further understand the mechanism of K562-induced NK cell expression of PD-L1, NK cells were FACS-purified to repeat the experiment of enriched NK cells. It was observed that PD-L1 was induced by specific interactions between K562 cells and purified NK cells (FIG. 1F). Experiments were undertaken to test whether direct cell contact was required for PD-L1 induction. For this purpose, NK cells were cultured in the supernatants from K562 cells alone or in the supernatants from K562 cells co-incubated with NK cells. The conditioned media marginally induced PD-L1, significantly less so when compared to NK cells directly incubated with K562 cells (FIG. 7B). K562 cells incubated in transwells did not induce PD-L1 on NK cells (FIG. 1G). Of note, PD-L1 expression could also be more modestly induced on CD8⁺ T cells and B cells when co-incubated with K562 cells, but not in NK-T cells or CD4⁺ T cell (FIG. 7C-7G). Collectively, these results show that direct interaction between NK cells and K562 myeloid leukemia cells alone is sufficient to promote PD-L1 expression on NK cells.

PD-L1 Expression Marks NK Cell Activation and Positively Correlates with Clinical Outcome of AML Patients

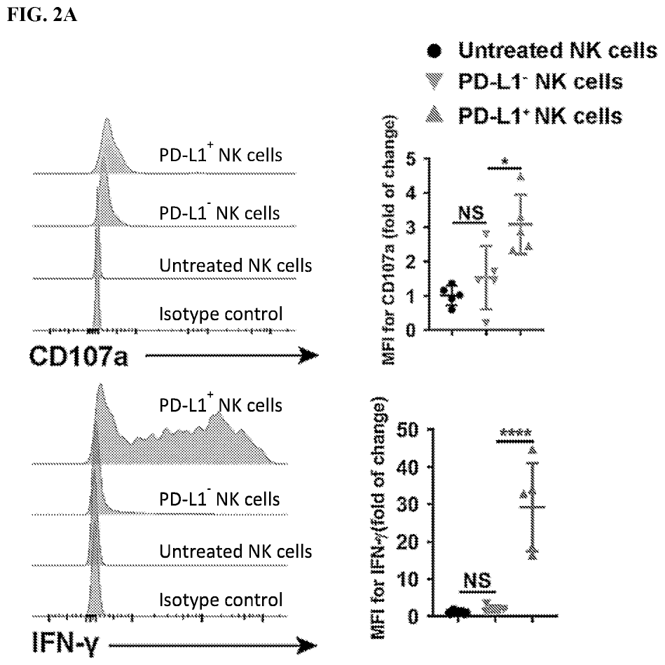

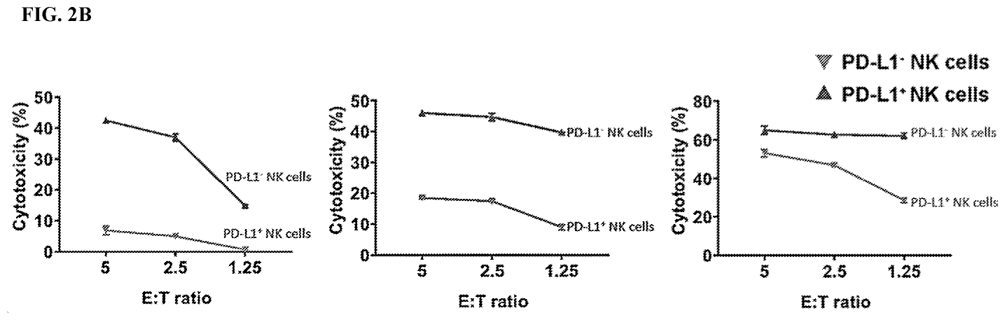

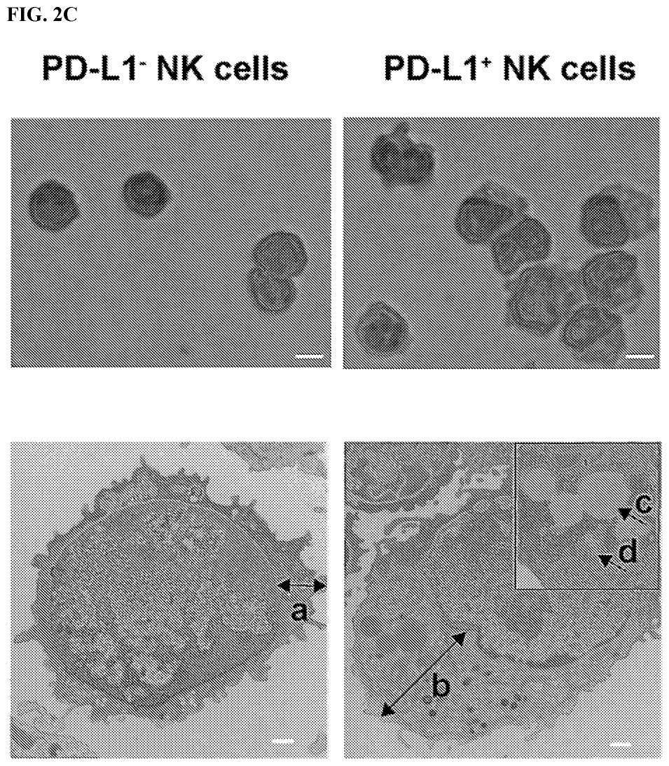

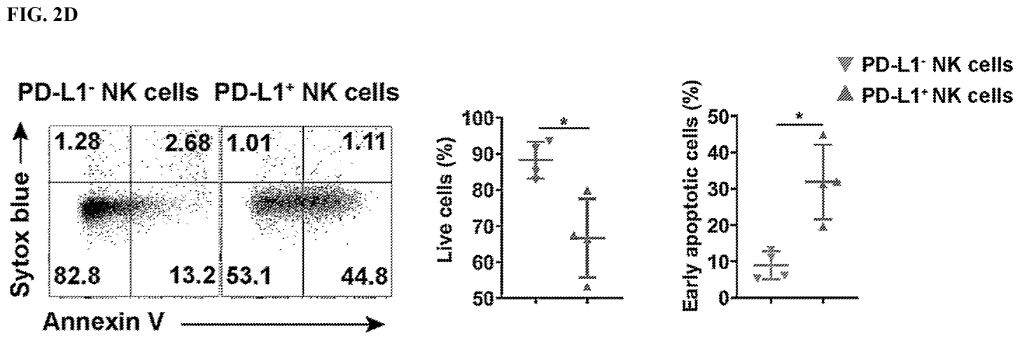

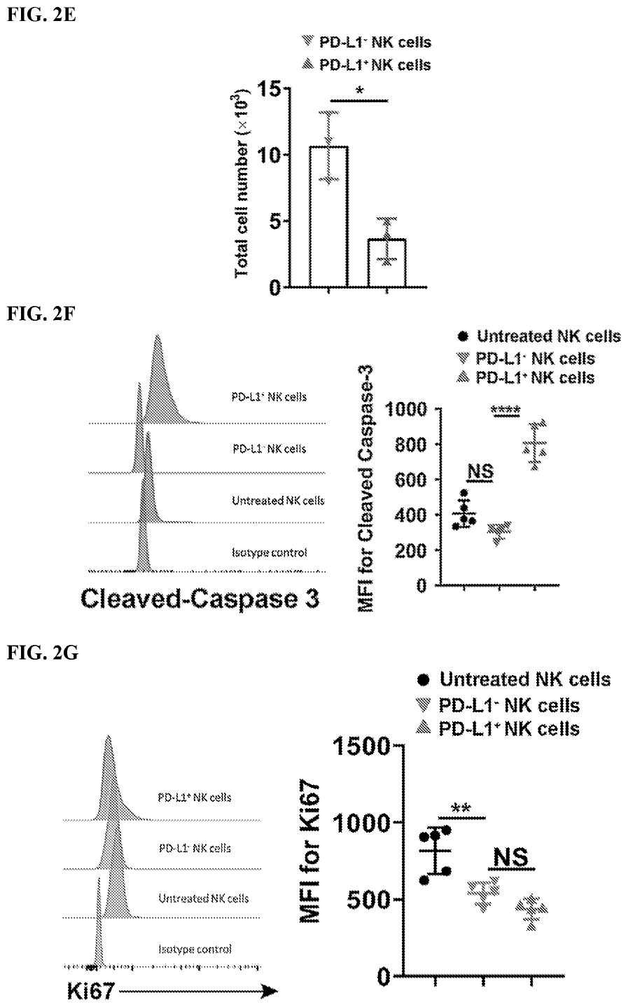

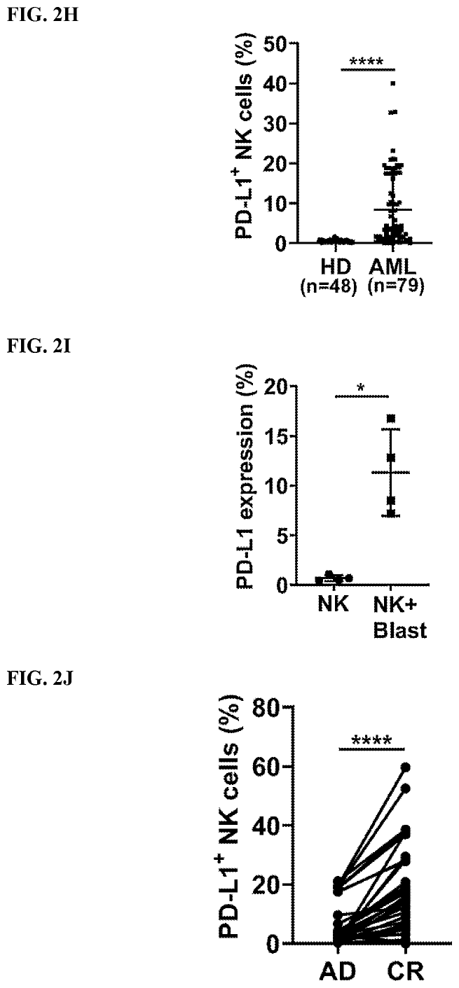

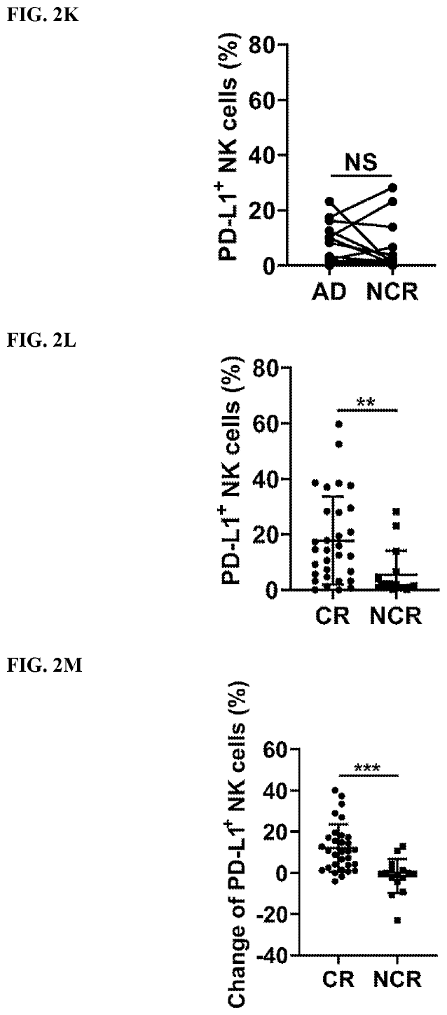

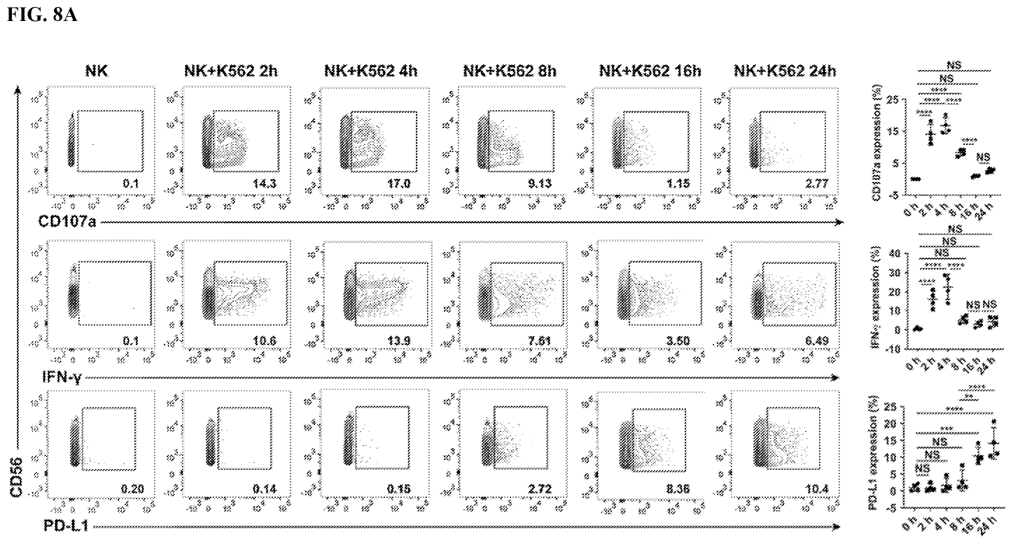

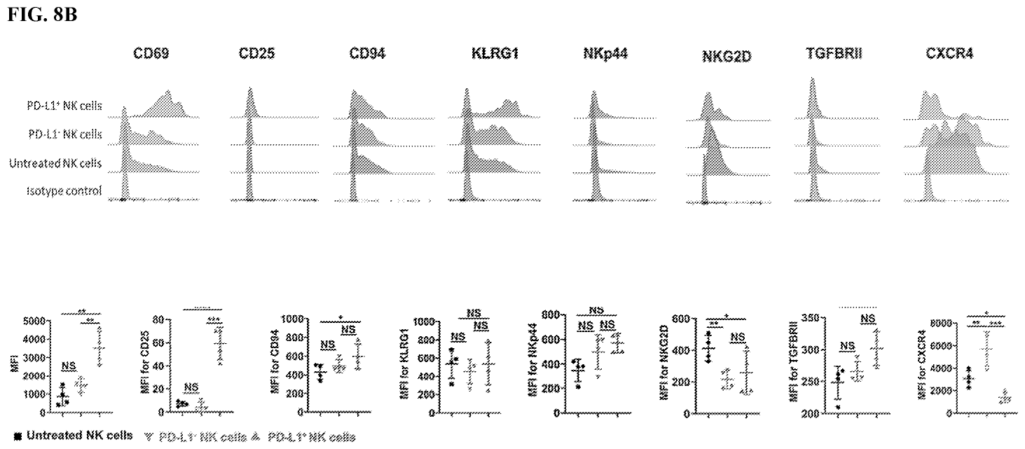

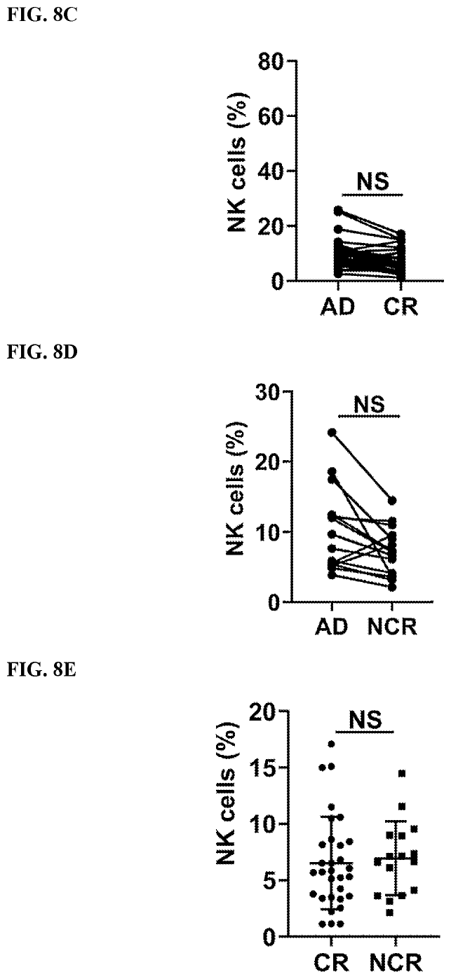

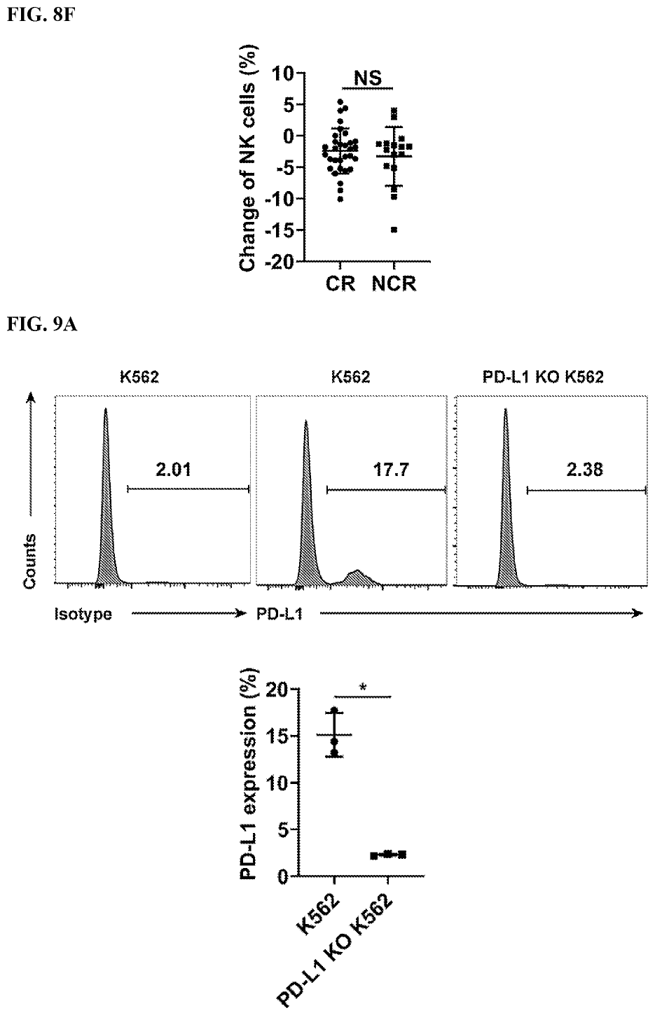

Next, the function of PD-L1 expression on NK cells was investigated. NK cell expression of CD107α and IFN-γ production are commonly used as functional markers for NK cell degranulation and cytokine production, respectively, following NK cell activation [see, for example, 17, 20]. While degranulation and IFN-γ production occurred within 2 hours of NK cells encountering K562 cells (FIG. 8A, top and middle panel), PD-L1 upregulation on NK cells did not significantly increased until 16 h (FIG. 8A, bottom panel). These data suggest that PD-L1 upregulation on NK cells might not be the driver of NK cell activation but rather the result of NK cell activation. The functional phenotype of PD-L1⁺ and PD-L1⁻ NK cells following co-culture with K562 cells were compared. Data showed that the expression of CD107α and that of IFN-γ were significantly increased in PD-L1⁺ NK cells compared to PD-L1⁻ NK cells (FIG. 2A). The ⁵¹Cr release assay confirmed the cytotoxicity of PD-L1⁺ NK cells was dramatically increased compared to PD-L1⁻ NK cells (FIG. 2B). These results further suggest that PD-L1⁺ NK cells are highly activated immune effector cells. Giemsa staining showed that PD-L1⁺ NK cells were larger in size and had a thicker cytoplasm (FIG. 2C, top panel). These observations of the PD-L1⁺ NK cells appearing larger and functionally more activated than the PD-L1⁻ NK cells were confirmed by transmission electron microscopy (FIG. 2C, bottom panel). The cytoplasm of freshly isolated PD-L1⁺ NK cells contains more mitochondria and liposomes, which may account for their larger size (FIG. 2C, bottom panel). The survival and proliferation capacity of PD-L1⁺ NK cells was also examined. Compared to PD-L1⁻ NK cells, PD-L1⁺ NK cells are more apoptotic (FIG. 2D-F). The proliferation in PD-L1⁻ NK cells and in PD-L1⁺ NK cells showed no significant difference (FIG. 2G). Using flow cytometry, the expression of specific surface markers present on these two NK cell subsets were measured. Experiments showed that the expression of the two activation antigens, CD69 and CD25, were significantly increased on PD-L1⁺ NK cells compared to PD-L1⁻ NK cells, while the receptors CD94, KLRG1, NKp44, NKG2D and TGFβRII did not show a significant difference in expression between PD-L1⁺ and PD-L1⁻ NK cells (FIG. 8B). CXCR4 expression was decreased on PD-L1⁺ NK cells compared to PD-L1⁻ NK cells, which could promote their egress from the bone marrow niche [See, for example, 21] (FIG. 8B). These data demonstrate that PD-L1 can be induced in NK cells when encountering tumor cells and compared to PD-L1⁻ NK cells, PD-L1⁺ NK cells appear to possess higher levels of effector functions against tumor cells.

Experiments were conducted to address whether PD-L1⁺ NK cells exist in cancer patients and whether this NK cell subset is correlated to standard chemotherapy outcomes. For this purpose, 79 patients with AML were recruited and results showed that the PD-L1⁺ NK cell population existed in the majority of AML patients but not in the healthy donors (FIG. 2H). The percentages of PD-L1⁺ NK cell population in the AML patients are up to 40% with 77% (61/79) of the patients expressing PD-L1 on NK cells above the level of healthy donors (FIG. 2H). The induction of PD-L1 on NK cells during the ex vivo incubation of AML blasts with NK cells from healthy donors was confirmed (FIG. 2I). When comparing the percentage of PD-L1⁺ NK cells at the time of evaluation for response to standard induction chemotherapy of two cycles, it was observed that AML patients who achieved complete remission (CR; n=31 of 47) had a significantly higher percentage of PD-L1⁺ NK cells at CR compared to the percentage of PD-L1⁺ NK cells at the time of diagnosis (FIG. 2J). In contrast, AML patients who did not achieve CR (n=16 of 47) showed no significant difference in percentage of PD-L1⁺ NK cells between the time of diagnosis and the time of assessment for CR (FIG. 2K). At the time of evaluating treatment responses post chemotherapy, AML patients who achieved CR had a significantly higher percentage of PD-L1⁺ NK cells compared to AML patients who did not achieve CR (FIG. 2L). When the data were reanalyzed and presented as percentage changes of PD-L1⁺ NK cells from the time at diagnosis to post chemotherapy, a significant difference was also observed between the patients with CR and those without CR (FIG. 2M). However, these differences were not observed in the percentage of total NK cells at diagnosis when compared to the time of CR evaluation regardless of whether or not the AML patients achieved CR (FIG. 8C-8F). These data suggest that the percentage of PD-L1⁺ NK cells at the time of CR evaluation is correlated with attainment of CR, rather than the percentage of total NK cells. Taken together, the data presented thus far suggest that the activated NK cells as identified by their expression of PD-L1 may possess anti-leukemic activity in vivo.

Targeting PD-L1 with the Humanized Anti-PD-L1 mAb Atezolizumab Enhances NK Cell Function

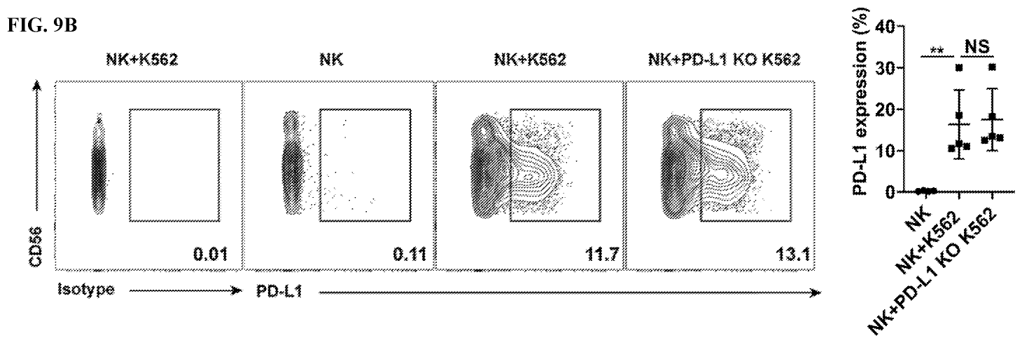

Results showed that PD-L1 expression or lack thereof could divide NK cells into two morphologically and functionally distinct populations with a higher level of cytotoxicity and IFN-γ production in the PD-L1⁺ subset compared to the PD-L1⁻ subset. To further evaluate the function of PD-L1 on NK cells, atezolizumab (AZ, trade name Tecentriq), one of the humanized mAb against PD-L1 that has been approved by the Food and Drug Administration for the treatment of non-small cell lung cancer (NSCLC) was used [see, for example, 22]. The K562 myeloid leukemia cells express a very low level of PD-L1 (FIG. 9A), consistent with a previous report [see 23], To ensure no contribution from PD-L1 expression on K562 cells, a PD-L1 knockout (KO) K562 cells using the CRISPR-Cas9 system was generated (FIG. 9A). Results showed that the lack of PD-L1 expression on K562 cells did not affect their ability to induce the expression of PD-L1 on NK cells (FIG. 9B).

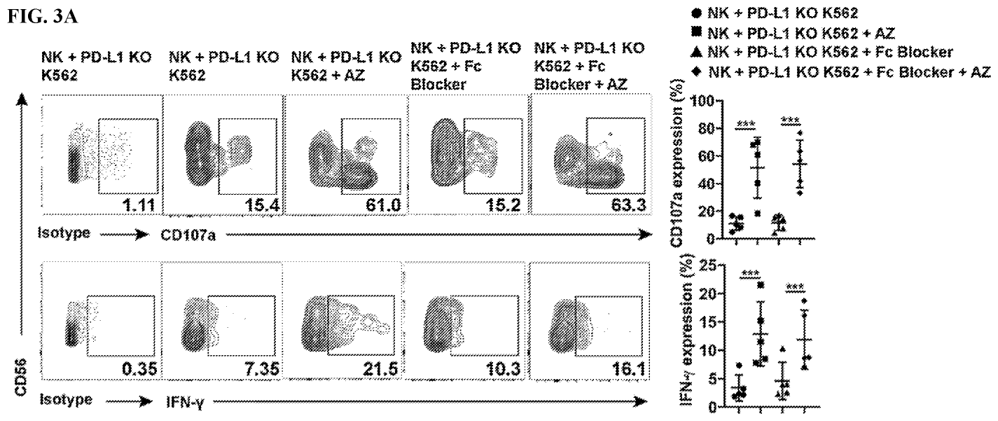

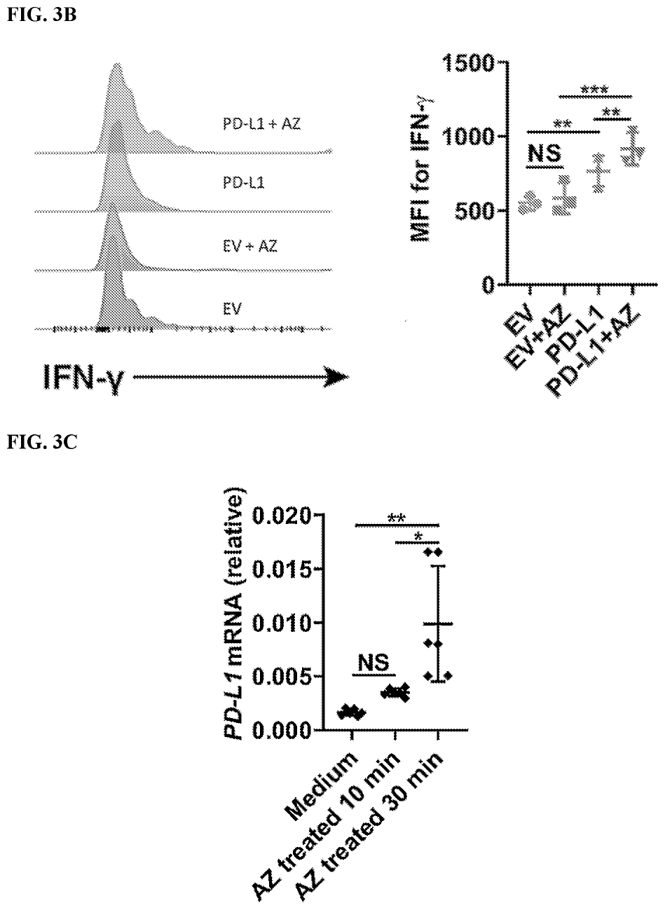

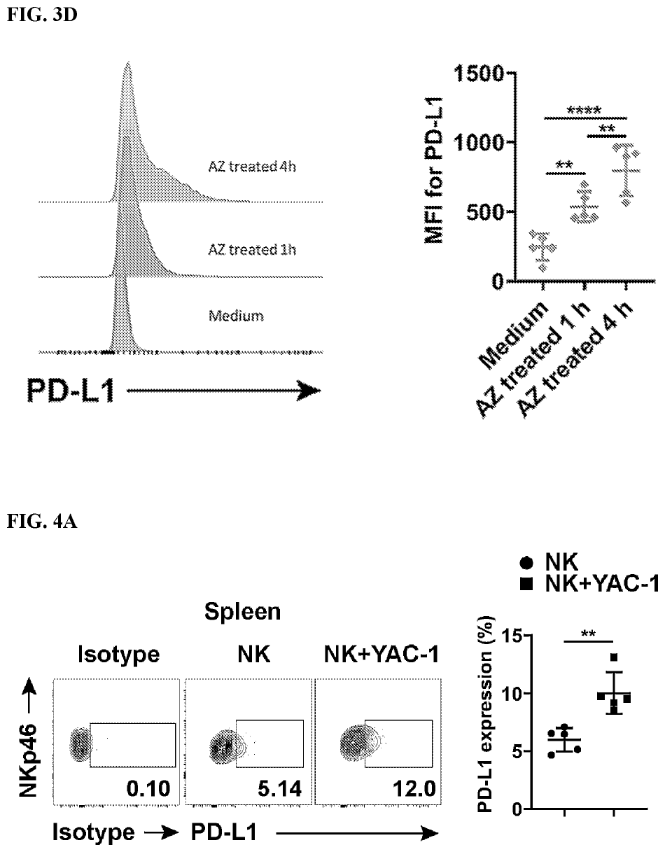

AZ is an IgG1 mAb engineered with a modification in the Fc domain that eliminates mAb-dependent cellular cytotoxicity (ADCC) [see 9]. Results showed that AZ treatment increased the expression of CD107a and IFN-γ in PD-L1⁺ NK cells in an ADCC-independent fashion (FIG. 3A), consistent with AZ's absence of an ADCC effect [9]. To further confirm the finding of PD-L1 positively regulating NK cell function, lentiviral transduction of NK cells with PD-L1 was undertaken. The PD-L1-transduced NK cells showed a significant increase in their IFN-γ production, compared to control-transduced NK cells (FIG. 3B), This effect was even more dramatically increased after treatment of the PD-L1-transduced NK cells with AZ (FIG. 3B).

In addition, results showed that PD-L1 expression of NK cells pretreated with K562 were further elevated at both the mRNA and protein levels in a time dependent manner after treatment with AZ (FIGS. 3C and D), suggesting that PD-L1 signaling by AZ induces continuously upregulating of PD-L1 expression that becomes available for additional activation by AZ.

Mouse PD-L1⁺ NK Cells Show Enhanced Antitumor Activity In Vivo

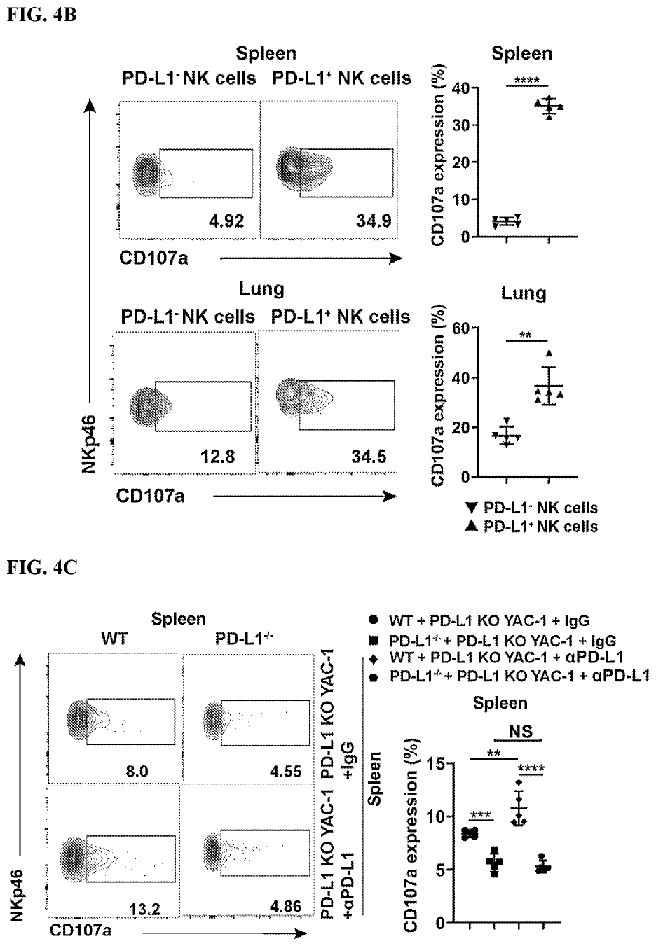

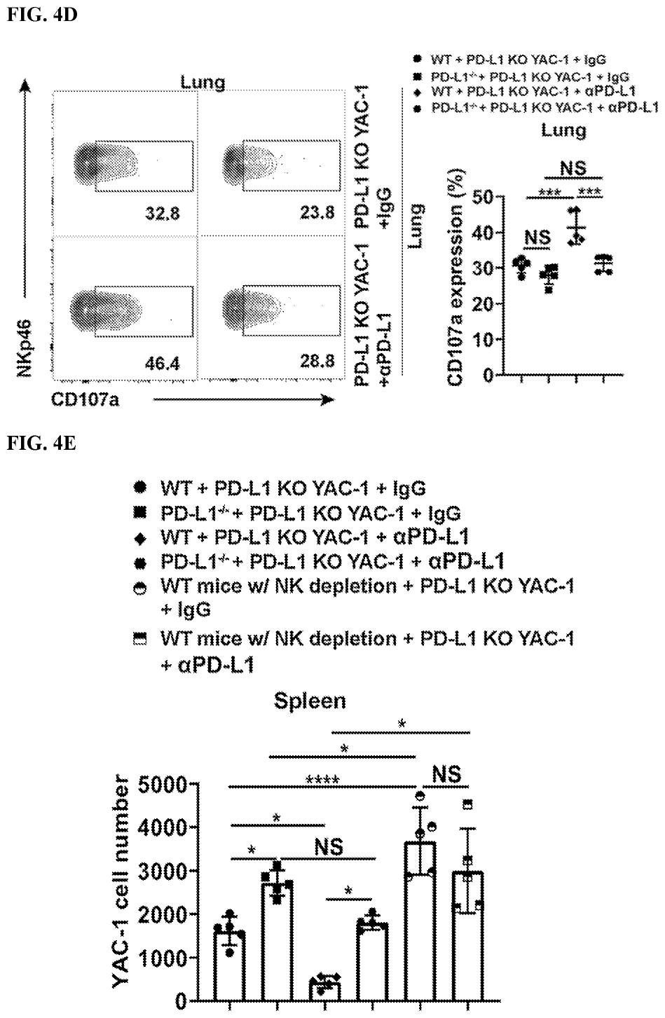

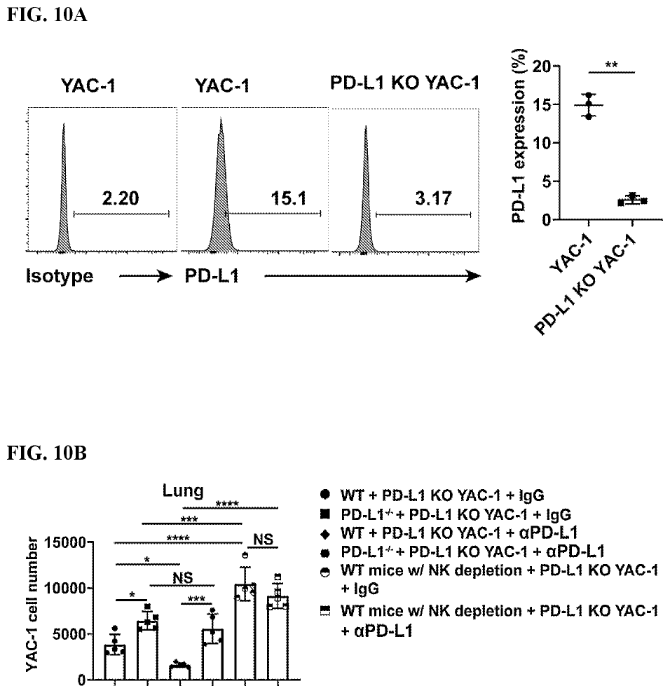

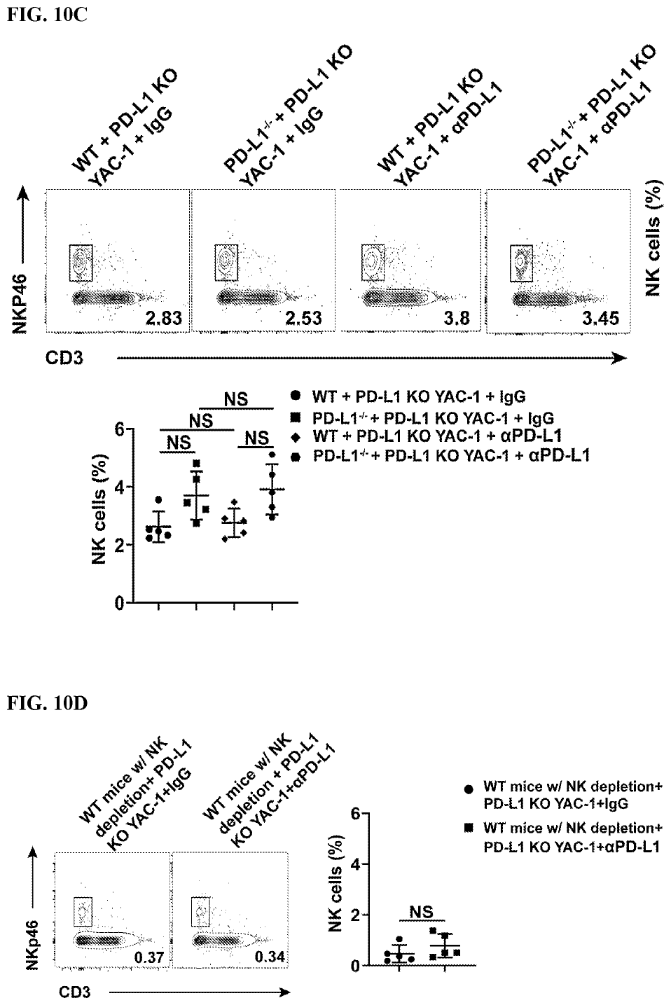

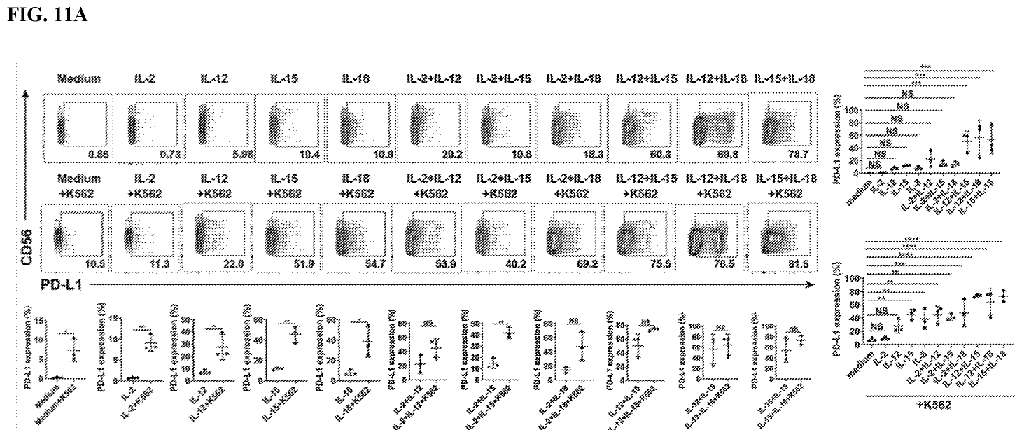

Experiments were conducted to test whether NK cells could be induced to express PD-L1 in the presence of tumor in animal models, and whether PD-L1⁺ murine NK cells display similar functional activity as seen thus far with human NK cells ex vivo. Results showed that mouse NK cells constitutively express PD-L1, which is consistent with a previous report [see 24]; results also showed that its expression could be significantly increased in mice bearing the lymphoid tumor YAC-1 (FIG. 4A). For further in vivo functional study, PD-L1 knockout YAC-1 cells (PD-L1 KO YAC-1) using the CRISPR-CAS9 system were generated (FIG. 10A). PD-L1+ NK cells in mice bearing PD-L1 KO YAC-1 tumors showed enhanced degranulation compared to their PD-L1⁻ NK cells (FIG. 4B). To further study the function of PD-L1 on mouse NK cells, PD-L1^{-/-} mice were used and results showed that CD107α expression was significantly decreased on splenic NK cells in PD-L1^{-/-} mice and showed a similar trend in lungs compared to NK cells in WT mice engrafted with the PD-L1 KO YAC-1 tumor cells (FIGS. 4C and 4D). In vivo anti-PD-L1 mAb treatment of mice bearing PD-L1 KO YAC-1 tumors increased CD107α expression on NK cells in WT mice but not on NK cells in PD-L1^{-/-} mice (FIGS. 4C and 4D). In wildtype (WT) mice with PD-L1^{+/+} NK cells implanted with PD-L1 deficient tumor cells, the tumor burden was significantly decreased when the PD-L1 antibody was used compared to IgG control-treated mice (FIG. 4E and FIG. 10B). This suggests that host cells' PD-L1 may play a positive role in controlling tumor development. However, for the similar experiment when PD-L1^{-/-} mice (i.e., lack of PD-L1 expression on NK cells) were used, results did not show significant antitumor activity of anti-PD-L1 antibody vs. IgG control (FIG. 4E and FIG. 10B). NK cell percentages did not change in tumor-bearing WT and PD-L1^{-/-} mice with or without PD-L1 mAb treatment (FIG. 10C). These data exclude the possible involvement of PD-1 (on T cells) and the mechanism appears to be the PD-1-independent mechanism. It was observed that tumor burden was lower in WT mice compared to PD-L1^{-/-} mice, which may indicate the effect of an-PD-L1 antibody was mediated by NK cells. NK cells were depleted in WT mice implanted with PD-L1 deficient tumor cells and the mice were treated with an anti-PD-L1 antibody or IgG control (FIG. 10D). Results showed that when NK cells are absent, there were no effects of anti-PD-L1 antibody (FIG. 4E and FIG. 10B), suggesting that NK cells play a role in mediating the effects of anti-PD-L1 antibody in our animal model. Together, these results suggest that PD-L1+ NK cells are essential for antitumor activity of the PD-L1 mAb in mice bearing PD-L1⁻ tumors, and that the antitumor effects of the mAb is acting directly on NK cells. The in vivo mouse studies herein suggest that the use of ant-PD-L1 mAb to target PD-L1⁺ NK cells can be a new strategy to be considered for cancer immunotherapy in the absence of a PD-L1+ tumor.

Anti-PD-L1 Antibody Augments Human PD-L1⁺ NK Cell Antitumor Activity In Vivo

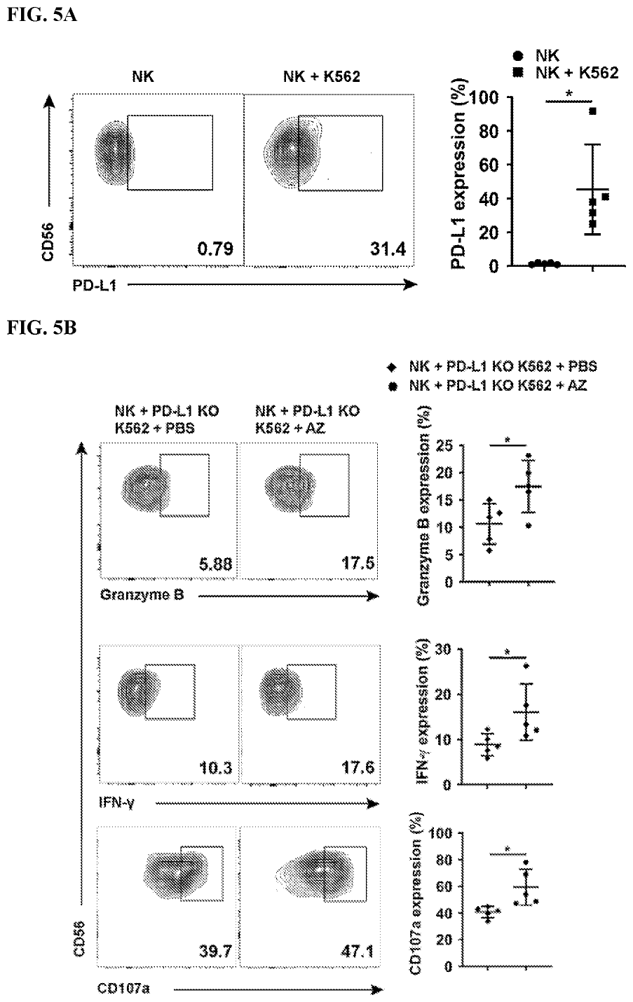

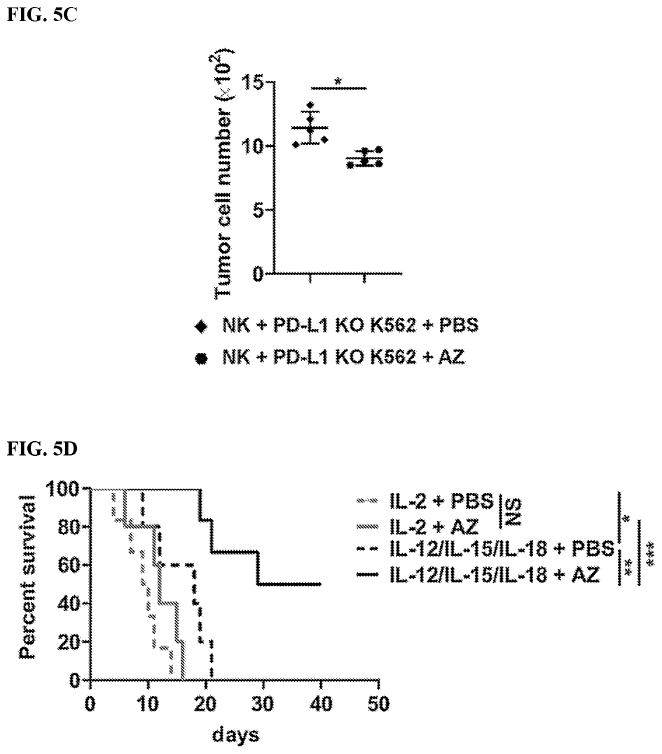

Having hypothesized from the AML patients' data that PD-L1⁺ NK cells could have antitumor activity in vivo (FIGS. 2L and 2M), and having shown an improved anti-tumor effect of mouse NK cells by delivering an anti-PD-L1 mAb against a malignant mouse tumor lacking PD-LI in vivo, experiments were conducted to reproduce this finding in an orthotopic mouse model using human NK cells and the K562 myeloid leukemia followed by in vivo delivery of the humanized anti-PD-L1 mAb AZ versus placebo (placebo was PBS and not isotype IgG, to avoid activating NK cell ADCC that is not active with AZ). For this purpose, human primary NK cells were transplanted into NSG mice, without or with PD-L1-KO K562 myeloid leukemia cells. Results showed that the presence of the PD-L1-KO K562 cells resulted in a very significant increase in the expression of PD-L1 on the human NK cells in vivo (FIG. 5A). Treatment of these mice with either a placebo or the AZ resulted in the latter group showing a significant increase in NK cell expression of granzyme B, IFN-γ, and CD107α when compared to the NK cells in the placebo-treated group (FIG. 5B). Further, results showed that the mice treated with AZ had a significantly lower tumor burden compared to the placebo-treated mice by enumerating the PD-L1-KO K562 myeloid leukemia cells upon sacrifice of the mice (FIG. 5C).

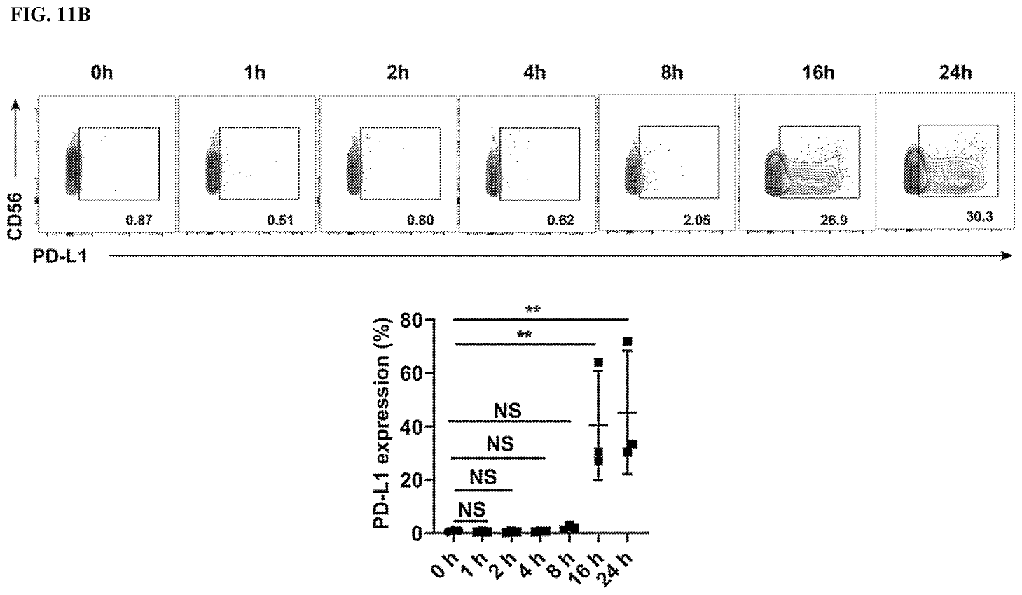

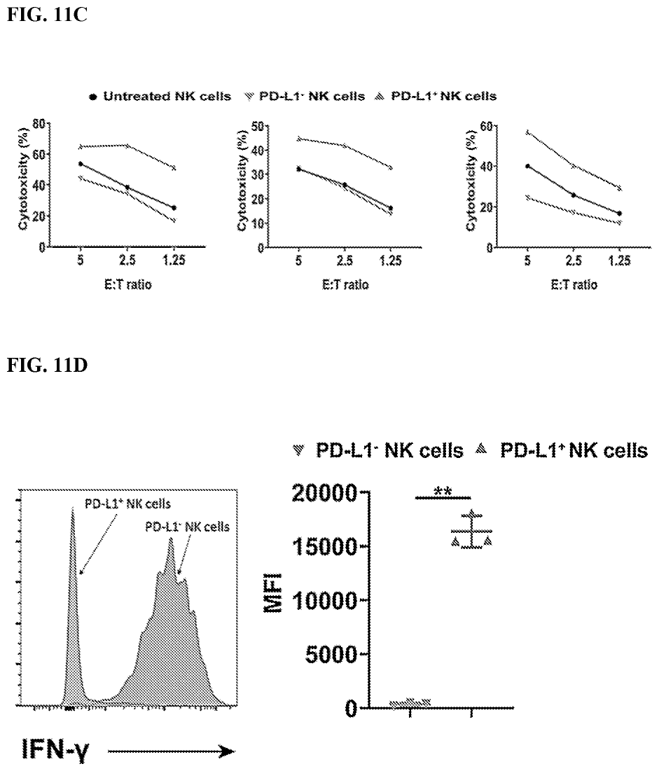

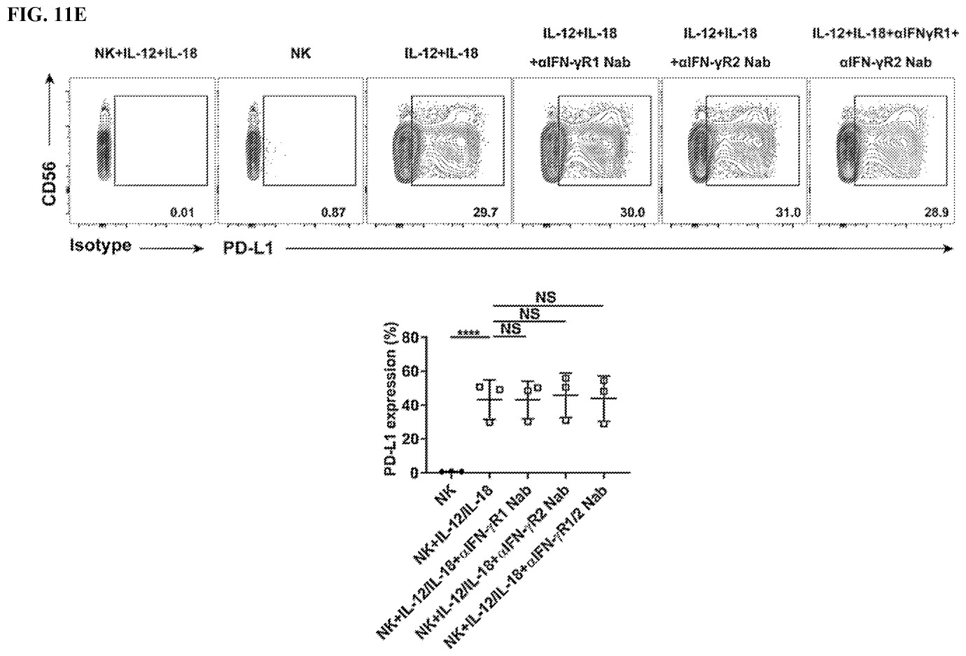

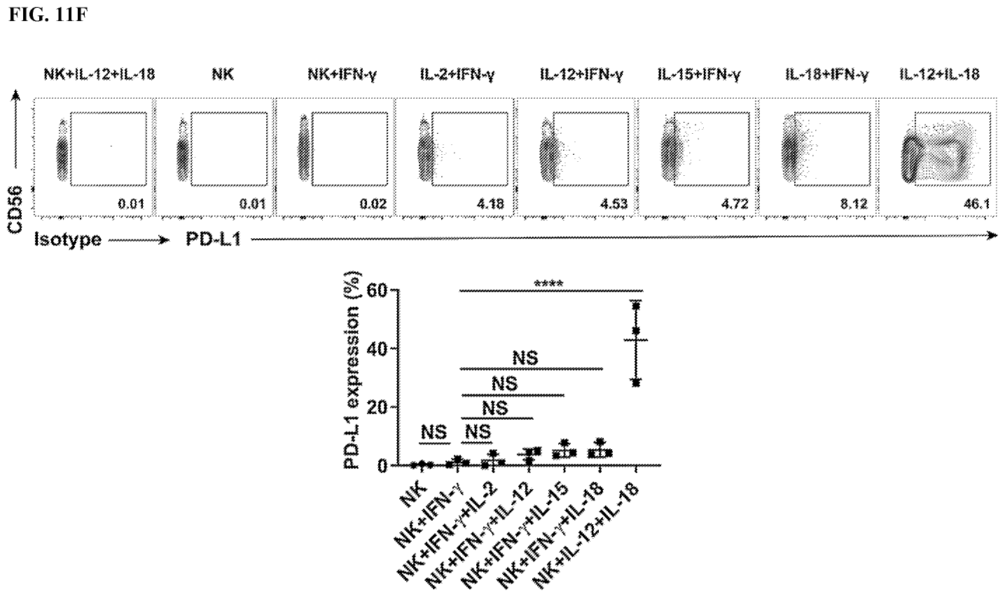

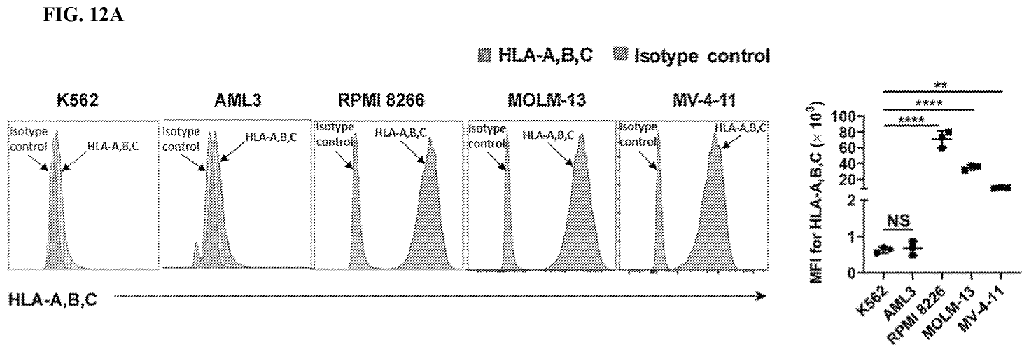

The effects of NK cell-activating cytokines on NK cell PD-L1 expression in the absence or presence of the PD-L1-KO K562 myeloid leukemia cells were assessed. Alone, IL-2 had essentially no effect on NK cell PD-L1 expression in the absence or presence of the K562 cells. In contrast, IL-12, -15 and -18 each had a significant effect on NK cell PD-L1 expression in the absence or presence of the K562 cells, and this was further increased when used in various combinations as shown in FIG. 11A. The PD-L1 expression kinetics were evaluated in culture with IL-12 and IL-18, one of strongest stimuli among all cytokines and their combinations that were tested. NK cell PD-L1 expression induced by IL-12 and IL-18 showed a pattern of induction that was similar to that seen in the setting of the K562 cells (FIG. 11B). NK cells with PD-L1 expression induced by culturing with IL-12 and IL-18 showed markedly higher levels of cytotoxicity and IFN-γ production compared to PD-L1⁻ NK cells and untreated NK cells (FIGS. 11C and 11D). IFN-γ is a potent inducer of PD-L1 expression in tumor cells [see, for example, 25]; however, blocking IFN-γ signaling did not affect NK cell PD-L1 expression induced by IL-12 and IL-18 despite both this combination of cytokines inducing massive amounts of IFN-γ in NK cells [for example, 26] (FIG. 11E). In addition, recombinant IFN-γ could not induce PD-L1 expression on NK cells alone or in combination with other cytokines (FIG. 11F). These results suggest that like tumor cells, cytokine combinations are also potent stimuli to induce PD-L1 expression on NK cells. It was hypothesized that the cytokine-induced PD-L1 on NK cells should respond to AZ treatment, providing a rationale for exploring the combination of NK-activating cytokines and AZ for the treatment of cancer.

In an attempt to assess the effect of treatment with the humanized anti-PD-L1 mAb AZ on survival of mice engrafted with human NK cells and the human PD-L1-KO K562 myeloid leukemia cells, mice were treated with various combinations of NK-activating cytokines in the absence or presence of AZ. As predicted from earlier in vitro work showing IL-2 alone did not increase PD-L1 expression on NK cells (FIG. 11A), neither the in vivo administration of IL-2 alone had much effect on survival, nor did IL-2 administered in combination with AZ, prolonged any survival beyond day 16 (FIG. 5D). The administration of combinations of IL-12 and IL-15 or IL-12 and IL-18 had substantial effects on increasing PD-L1 expression on NK cells in vitro (FIG. 11A), but the combination of these three cytokines in the absence of AZ only modestly improved survival over IL-2 plus AZ, with no survival beyond day 21 (FIG. 5D). In distinct contrast, when mice were treated with IL-12, IL-15 and IL-18 in combination with AZ, the mice showed a significant improvement in survival with 40% of mice alive at day 40 (FIG. 5D). These data, together with those in FIG. 4E, implied that anti-PD-L1 mAb AZ directly acts on PD-L1+ human NK cells in combination with three NK-activating cytokines to significantly prolong survival in mice engrafted with a lethal dose of human myeloid leukemia.

The PI3K/AKT Signaling Pathway Regulates PD-L1 Expression on NK Cells

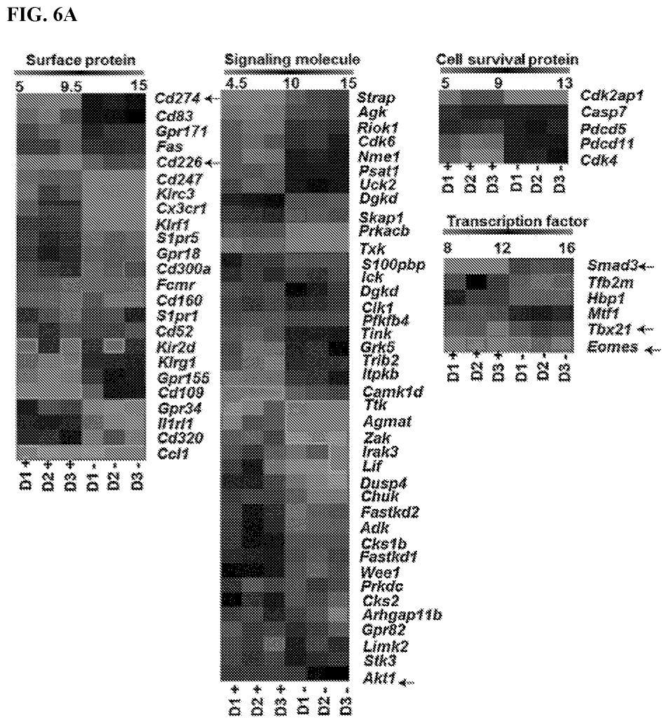

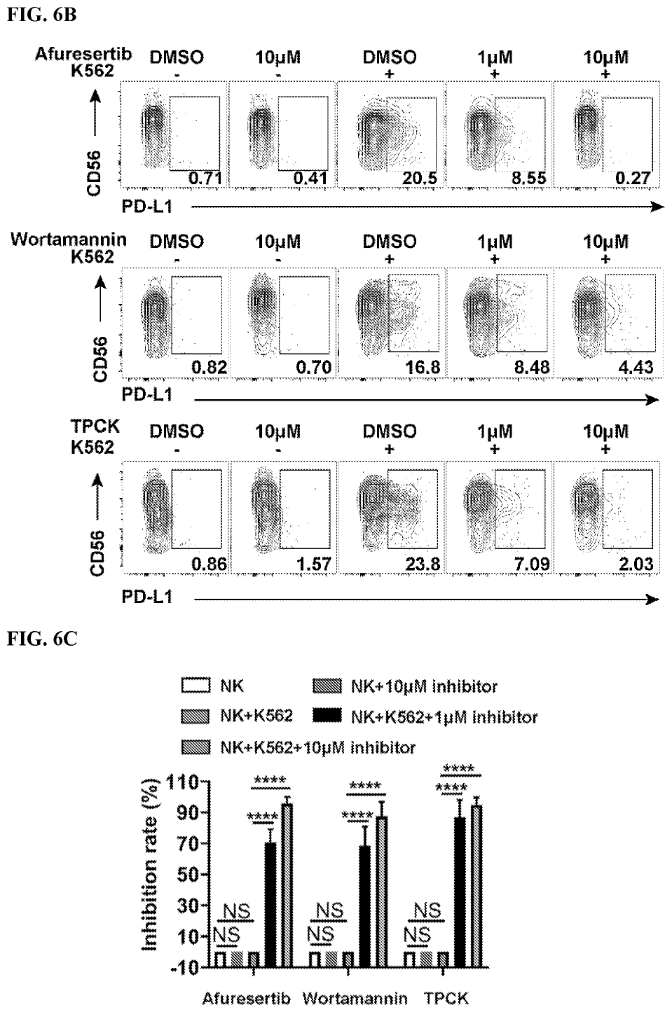

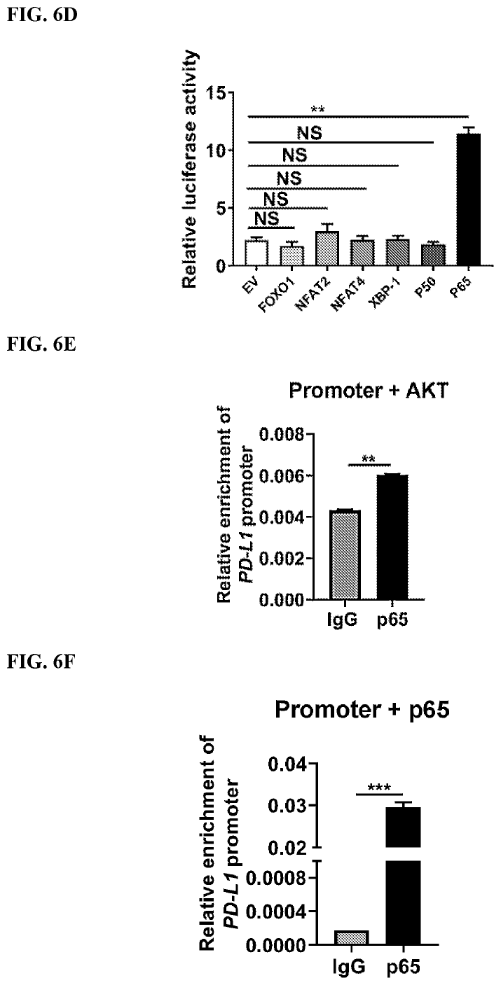

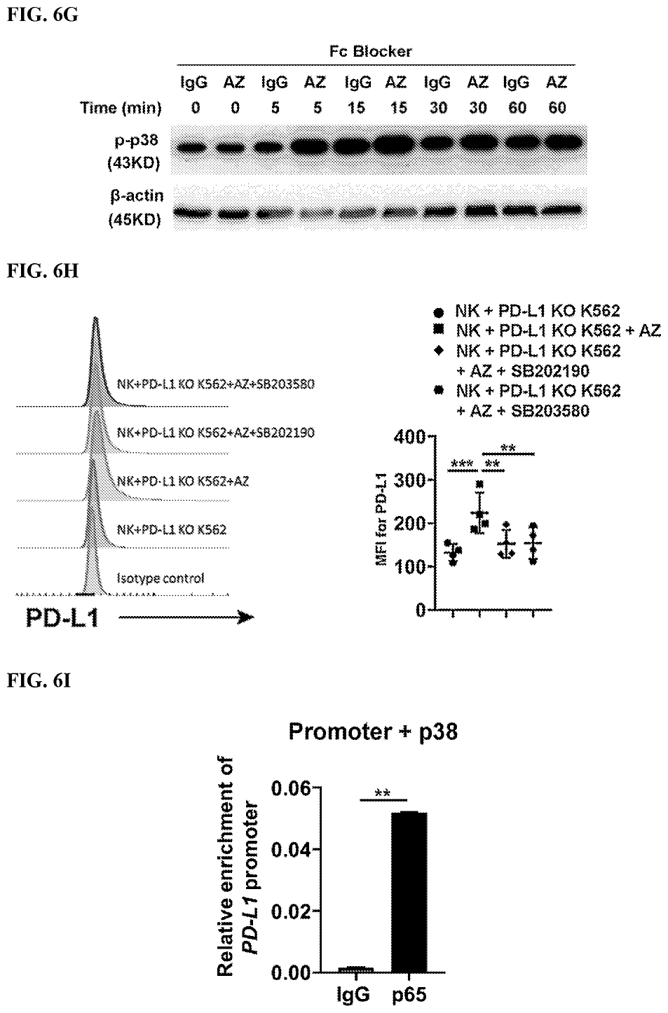

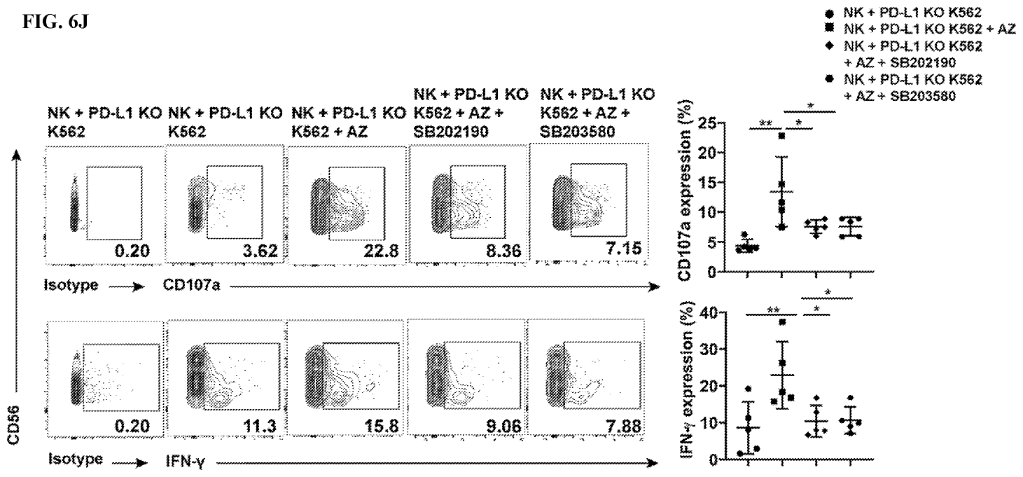

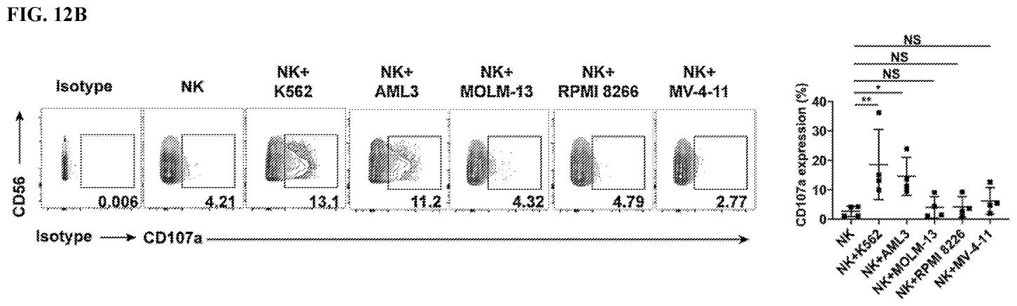

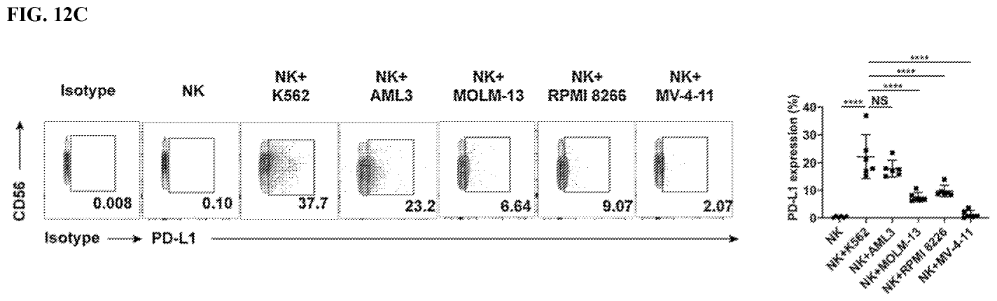

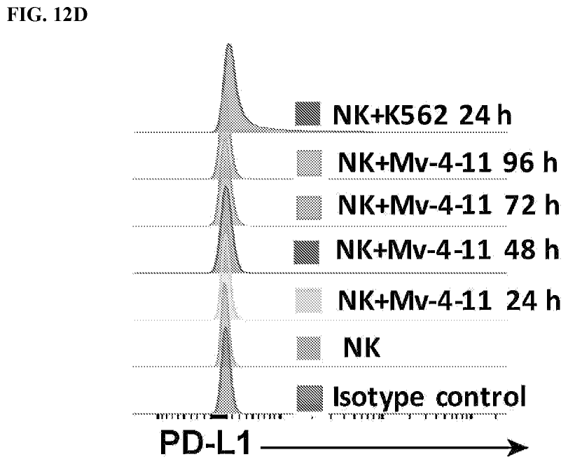

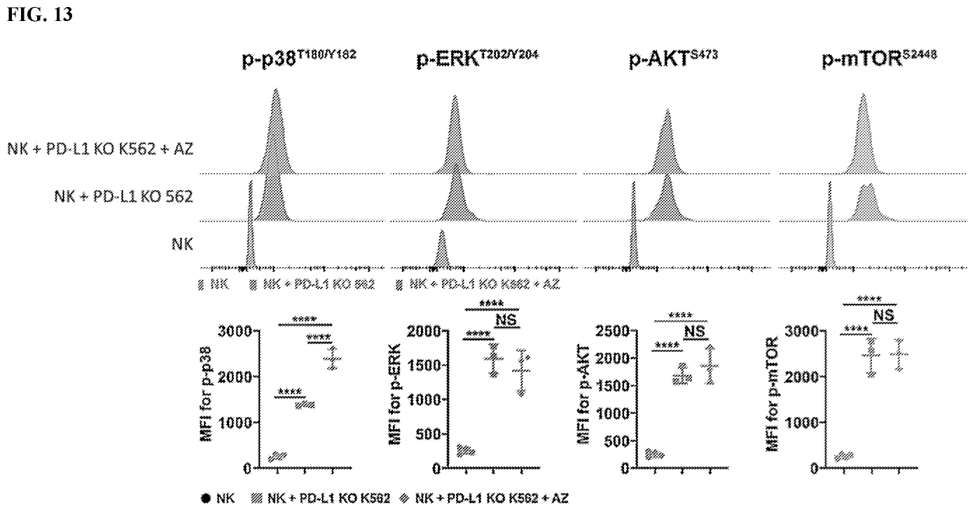

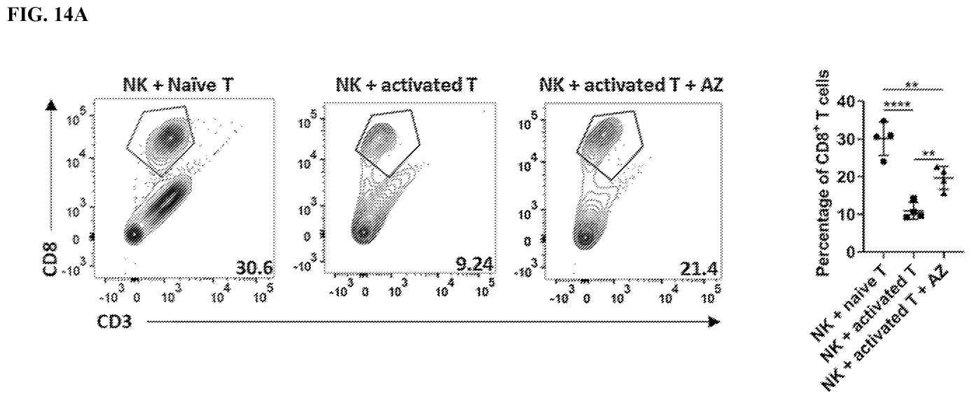

To investigate mechanisms by which PD-L1 is induced in NK cells by myeloid leukemia cells, an RNA microarray was performed to profile gene expression in PD-L1⁺ NK cells vs PD-L1⁻ NK cells, both of which were FACS-purified from bulk NK cells after being co-cultured with K562 cells. The results showed that the PD-L1⁺ NK cell subset had higher expression levels of TBX21 and EOMES, which are the two signature transcriptional factors required for NK cells to gain functional maturity [for example 27, 28]. CD226, an activation marker for NK cells [for example 29], had higher expression in PD-L1⁺ NK cells while the negative regulatory transcriptional factor, SMAD3, had lower expression levels in PD-L1⁺ NK cells [for example 30] (FIG. 6A). These gene expression patterns indicate that PD-L1⁺ NK cells exert unique gene profiling compared to their PD-L1⁻ counterpart and are consistent with our above characterization showing that PD-L1⁺ NK cells are an activating NK cell subset. In addition, the microarray data implied that protein kinase B (AKT) signaling was involved in regulating PD-L1 expression on NK cells (FIG. 6A). The protein kinase B (AKT) family contains three members AKT1, AKT2, and AKT3 [for example 31]. To further confirm whether AKT signaling regulates PD-L1 expression on NK cells, a global AKT inhibitor against AKT1/2/3 (afuresertib) was used to pretreat NK cells. They were subsequently incubated with K562 myeloid leukemia cells and then PD-L1 expression on NK cells was measured. Treatment with afuresertib significantly reduced PD-L1 expression (FIGS. 6B and 6C), suggested that global AKT inhibition is capable of blocking PD-L1 expression. Upstream of the AKT cascade is phosphatidylinositol-3-kinase (PI3K). Treatment with the PI3K inhibitor wortmannin also significantly reduced PD-L1 expression on NK cells when incubated with K562 myeloid leukemia cells (FIG. 6B, top and middle panels, and FIG. 6C). Experiments were conducted to identify which transcription factor(s) downstream of PI3K/AKT signaling regulate PD-L1 expression in NK cells. A PD-L1 promoter was cloned 2.1 kb upstream of the transcription start site, co-transfected with genes for specific transcription factors in 293T cells, and the activity of the PD-L1 promoter was measured by luciferase assay. Results showed most transcriptional factors of the PI3K/AKT cascade including XBP-1, FOXO-1, NFAT-2, and NFAT-4 did not activate the PD-L1 promoter [for example, 32-34]; however, the PI3K/AKT downstream transcription factor p65 enhanced the PD-L1 promoter activity 5-fold compared to the control (FIG. 6D). The p65 subunit comprises part of the nuclear factor kappa B (NF-κB) transcription complex, which plays a crucial role in inflammatory and immune responses [for example 35]. The regulation role of p65 in PD-L1 expression was confirmed using a specific p65 inhibitor TPCK (FIG. 6B, bottom panel, and FIG. 6C). To validate the role of the PI3K/AKT/p65 pathway in regulating PD-L1 expression, the binding of p65 with the PD-L1 promoter was examined. For this purpose, the PD-L1 promoter was co-transfected with AKT or p65 in 293T cells. Results showed that both introduction of AKT and p65 both can enhance the association of p65 on the PD-L1 promoter compare to empty vector control (FIGS. 6E and 6F, FIG. 13). These results collectively show that signaling through PI3K/AKT/NF-κB plays a critical role in regulating PD-L1 expression in NK cells. Since NK cell activation is usually triggered by recognizing the missing MHC-I class molecules [for example 15], experiments were conducted to investigate if the susceptibility of NK cells to tumors is associated with PD-L1 expression on NK cells. For this purpose, the expression of HLA-A, B, C was examined on the target cell lines and found that K562 and AML3 cells have significantly lower expression of HLA-A, B, C, both of which strongly activate NK cells and induce PD-L1 expression (FIG. 12A-12C). In contrast, RPMI 8226, MOLM-13 and MV-4-11 cells with the high levels of HLA-A, B, C expression could not efficiently activate or induce PD-L1 expression (FIG. 12A-12C), even for extended incubation time (FIG. 12D). These data suggest that target susceptibility to NK cell cytotoxicity may be associated with the capacity of PD-L1 induction by tumor cells.

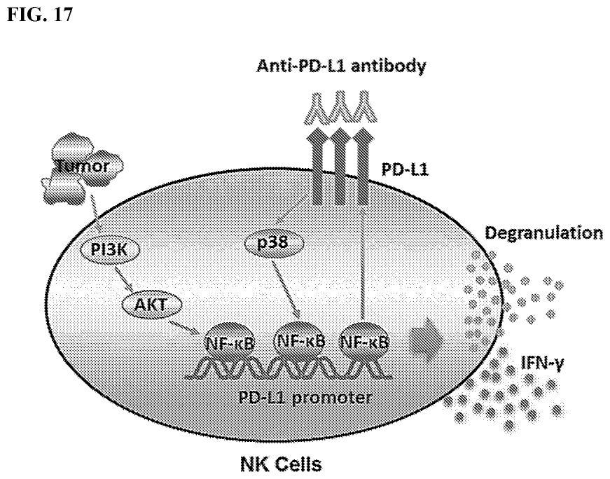

The above experiments reveal that tumor cells induce PD-L1 expression on NK cells via the PI3K/AKT/NF-κB pathway (FIG. 17). Results showed that the binding of AZ to tumor-induced PD-L1 on NK cells led to further NK cell activation (FIG. 3A), suggesting that the induced PD-L1 on NK cells may signal to AZ treatment. Further experiments were conducted to explore the molecular mechanism(s) downstream of the PD-L1/AZ interaction in NK cells. For this purpose, four kinases that have been reported downstream of PD-L1 signaling in other cells were screened. Intracellular flow cytometry analysis in FIG. 13 showed that the level of p38, a kinase in regulating NK cell function and anti-tumor activity [for example 36], was increased by AZ treatment, while the levels of p-ERK, p-AKT, or p-mTOR were not increased. These data were confirmed by immunoblotting (FIG. 6G). Interestingly, it was previously reported that PD-L1 signaling in PD-L1⁺ tumor cells is via the PI3K/AKT pathway [for example 37]; however, the data herein showed that PD-L1 signaling in PD-L1⁺ NK cells was not via the PI3K/AKT pathway in the presence of AZ (FIG. 6G and FIG. 13A). The assays with the p38 inhibitors, SB203580 and SB202190, were found to reduce AZ-induced PD-L1 expression in the presence of K562 tumor cells, suggesting that the positive regulatory effect of p38 signaling in this setting (FIG. 6H). Consistent with this, ChIP assay results also showed that p38 signaling induced the binding of p65 to the PD-L1 promoter (FIG. 6I, FIGS. 13B and 13C). Functionality assays demonstrated that the two p38 inhibitors, SB203580 and SB202190, also inhibited AZ-induced CD107α and IFN-γ expression of NK cells in the presence of K562 tumor cells (FIG. 6J).

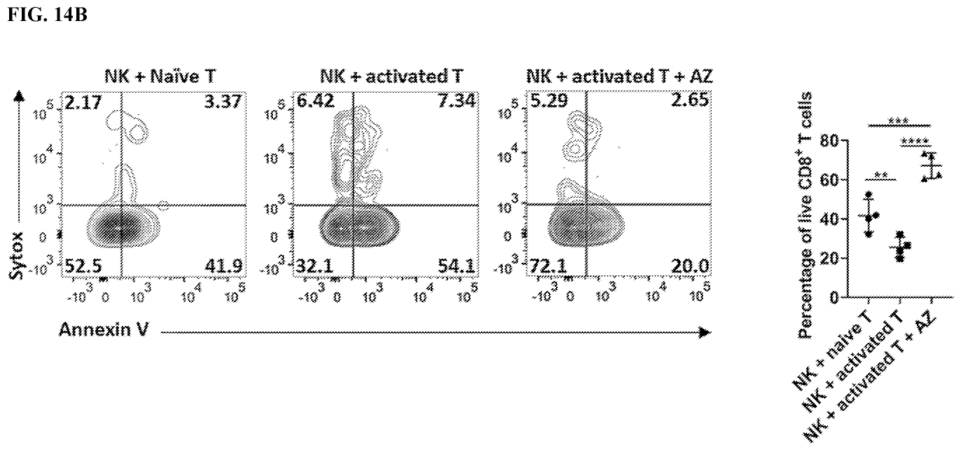

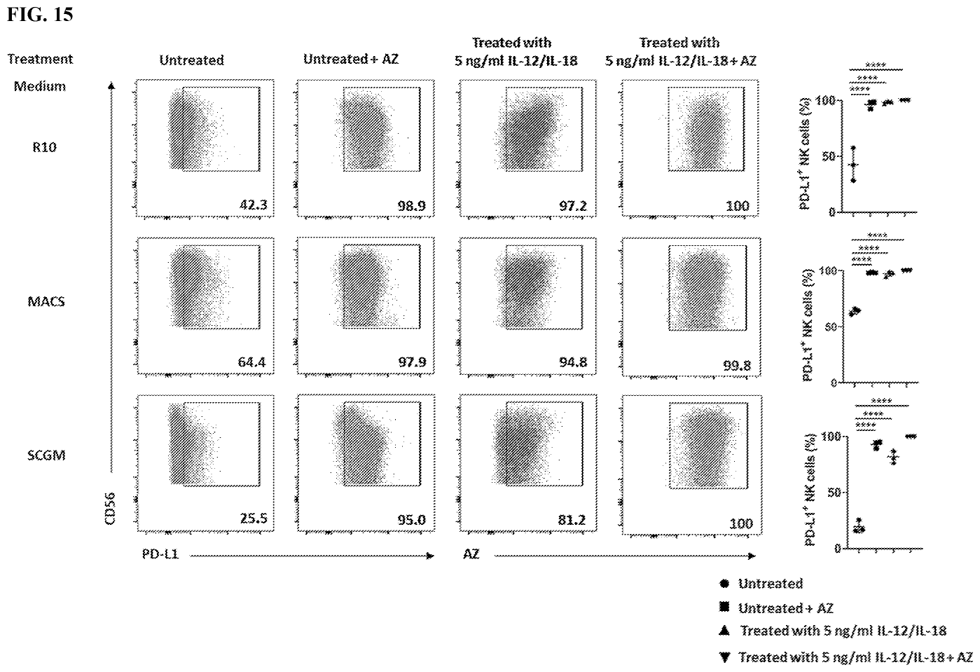

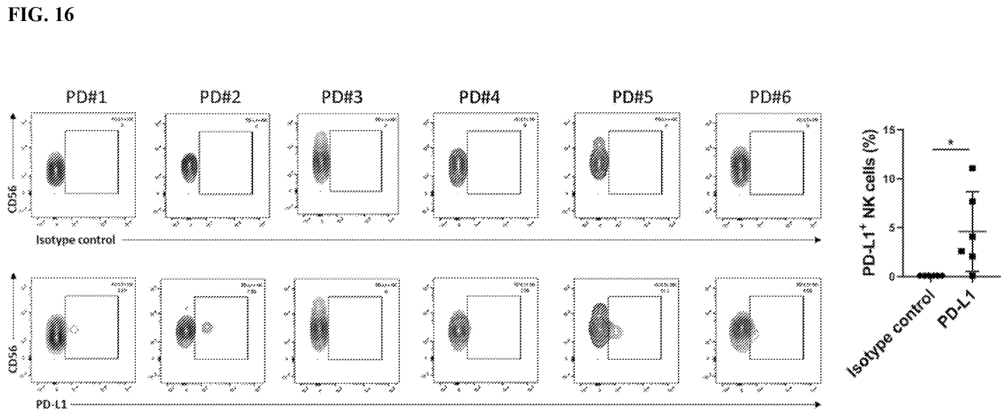

FIG. 14 shows the percentage of CD8⁺ T cells as examined by flow cytometry and shows the apoptosis of CD8⁺ T cells as examined by flow cytometry. Human primary NK cells were expanded by adding the K562 feeder cells (feeder cells were K562 cells with membrane-bound IL21 (62), treated with 100 Gy radiation) in the presence of 10 ng/ml IL-2. Expanded human primary NK cells for 7 days with indicated medium (R10: RPMI1640+10% FBS; MACS: MACS medium+5% human serum; SCGM: SCGM medium+5% human serum) were treated with or without 5 ng/ml IL-12 and IL-18 for 20 h in the presence of AZ or not. The expression of PD-L1 were examined by flow cytometry (FIG. 15). PBMC from lung cancer patients were isolated and examined for PD-L1 expression by flow cytometry (FIG. 16).

Taken together, the data herein suggest a model involving PD-L1 upregulation on activated NK cells via the PI3K/AKT signaling pathway after NK cells and tumor cells encounter with each other (FIG. 17); the model also involves the subsequent anti-PD-L1 binding to the upregulated PD-L1 on NK cells by tumor cells and further activation of NK cells via the p38 signaling; both events lead to NF-κB activation, resulting in a positive feedback loop to continuously induce PD-L1 expression and to activate NK cells. In the loop, the engagement of the anti-PD-L1 antibody with PD-L1 upregulates PD-L1 expression on NK cell surface, providing more binding sites for anti-PD-L1 mAb that could lead to continuous expression of p38, which further transduce stronger activation signaling to NK cells to maintain the cytotoxic and cytokine secretion features of NK cells.

Discussion

PD-L1 is typically expressed on tumor cells, allowing them to suppress PD-1⁺ T cell function thereby enhancing the tumor's ability to evade the immune system [for example 38]. However, the functional consequences of PD-L1 expression on NK cells and the role of PD-L1⁻ NK cells in regulation of the immune response have not been previously characterized. Experiments herein studied PD-L1⁺ and PD-L1⁻ NK cells in both human and mice in the setting of PD-L1⁻ tumors. PD-L1⁺ NK cells were found to have significantly enhanced cytotoxicity and IFN-γ production compared to PD-L1⁻ NK cells. Upon engagement with AZ, PD-L1 is able to further modulate NK cell function through the p38 signaling pathway, thus serving as a functional activation antigen for NK cells. Results showed that a significantly greater fraction of PD-L1⁺ NK cells at the time of CR evaluation compared to the time of diagnosis correlates with attainment of CR, while the total percentage of NK cells at these time points does not correlate with an improved clinical response. Finally, results demonstrated that infusion of AZ in combination with NK cell-activating cytokines significantly improves overall survival in an orthotopic mouse model of human NK cells and PD-L1⁻ K562 myeloid leukemia. Thus, not only do PD-L1⁺ NK cells correlate with disease response to therapy, their presence also provides a possible therapeutic opportunity for improved clinical outcome by using anti-PD-L1 mAb for PD-L1⁻ tumors via a NK cell activation pathway that is independent of T cells and PD-1.

T cells infiltrating into the TME are heterogeneous, contain both effector and bystander CD8⁺ T cells populations [for example 39, 40]. The higher percentage of effector T cells in the TME, the better the prognosis, and vice versa. However, there are fewer studies of NK cells within the TME, and little is known about PD-L1⁺ NK cells. Results herein showed that encountering myeloid leukemia cells, a proportion of NK cells lost most of their cytotoxic activity and became “bystander like” with little or no expression of PD-L1; while a second fraction of NK cells exposed to the same myeloid leukemia cells showed strong induction of PD-L1 expression, a state of activation and developed enhanced effector function toward tumor target cells. The more sensitive the target cell is to NK cytotoxicity and the more direct cell-cell contact of the NK cells with the target cells, the higher was the expression of PD-L1 and the stronger was the activation of the NK cell.

PD-1 expression on NK cells results in a negative regulatory event upon engagement with its ligand [see for example 41-43] as is well known in T cells [see for example 38, 44]. Previous functional analysis indicates that compared to PD-1⁻ NK cells, PD-1⁺ NK cells are less activated with a lower level of degranulation and impaired cytokine production upon their interaction with tumor targets [see for example 43]. In contrast, the study herein showed that PD-L1⁺ NK cells are more activated compared to their PD-L1⁻ counterparts upon their interaction with tumor targets. Results also showed that PD-L1 signaling is a positive regulatory event for NK cells upon engagement of anti-PD-L1 antibody or its ligand PD-1 and the p38/NF-κB signaling pathway, well known signaling important in regulating the function of NK cells [for example 32, 36], involves downstream cell activation of PD-L1⁺ NK cells. Activation of this PD-L1 signaling pathway in NK cells resulted in further expression of PD-L1, which, in the presence of excess anti-PD-L1 mAb further increased p38 signaling. This positive feedback loop continually provides intracellular signaling that allows the NK cell to retain an activated effector state (FIG. 17). Both effects are very likely to contribute to the potent anti-tumor effect seen by NK cells in both the in vitro and in vivo modeling performed in this study. Importantly, it was discovered that the induction of PD-L1 expression following the NK cell interaction with a tumor cell target susceptible to NK killing is via a PI3K/AKT signaling pathway, distinct from the p38 signaling pathway that mediates the effect resulted from the interaction between tumor cell-induced PD-L1 and its antibody.

Thus far, in immunotherapy, reversal and even enhancement of T cell anti-tumor activity through checkpoint blockade has had great success in the cancer clinic [for example 45-47]. It is therefore not unreasonable to believe that NK cell-based anti-tumor activity in the TME could also be reversed and enhanced. Results herein show that anti-PD-L1 mAb therapy enhanced PD-L1⁺ NK cell function against PD-L1⁻ tumors in both mouse and human systems in vitro and significantly improved survival against a PD-L1⁻ tumor in a PD-1-independent fashion in vivo. Further, in the mouse model, results demonstrate that depletion of NK cells or performing the same in vivo experiment in PD-L1 KO mice significantly decreased the anti-tumor effect of anti-PD-L1 mAb therapy, suggesting that the anti-tumor effects are mediated by the NK cells themselves following PD-L1 signaling within the TME. Results herein reveal a new strategy for an increased and prolonged immune response of NK cells in the TME, and provides an explanation on how immune therapy with anti-PD-L1 mAb can be effective in individuals whose tumors lack PD-L1 expression [8, 9].

IL-12, IL-15 and IL-18 cytokines are known to activate and expand NK cells, and each has been investigated in clinical studies [for example 48-51]. IL-12 has demonstrated antitumor effects through its regulation of both innate and adaptive immune cells [for example 52]. Recombinant human IL-15 has entered phase I/II clinical trials for treating various types of cancer [for example 53]. IL-15 has shown promising antitumor effects either when used alone or in combination with other treatments [for example 54, 55]. IL-18 also plays an important role in expansion and priming of NK cells [for example 56, 57]. The study herein study showed that the anti-PD-L1 mAb AZ had a significantly enhanced anti-tumor effect when administered in combination with these NK cell-activating cytokines, leading to a prolonged survival in mice engrafted with human NK cells and human myeloid leukemia, likely through the enhancement of NK cell function.

Together with the clinical correlative data presented on 79 AML patients in this report, the in vitro and in vivo studies herein suggest that for select AML patients with a significant fraction of PD-L1^{hi/+} NK cells at CR, an anti-PD-L1 mAb clinical trial at the time of CR can be considered likely in combination with an NK-activating cytokine such as IL-15. In addition to assessing NK cell function against autologous patient blasts pre- and post-anti-PD-L1 mAb administration, the time to relapse would be an additional important clinical endpoint to measure. This is particularly relevant when considering a highly vulnerable population such as elderly AML patients where the vast majority relapse within 2 years [for example 58].

In summary, the study herein identified a novel and unique subset of NK cells characterized by surface expression of PD-L1 in a fraction of cancer patients, and results were reproduced with both in vitro and in vivo tumor modeling. Data showed that binding of anti-PD-L1 mAb to PD-L1⁺ NK cells induced strong antitumor activity in vitro and in vivo that was independent of the PD-1/PD-L1 axis well-known in therapy with immune checkpoint inhibitors. These anti-tumor effects were shown to be dependent on both NK cells and their expression of PD-L1 and were effective against tumors lacking expression of PD-L1. Collectively, the study would suggest that the presence of PD-L1⁺ NK cells are associated with a favorable response following induction chemotherapy for AML, and the experimental data suggest these PD-L1+ NK cells can be further activated in vivo for an additional anti-tumor effects, likely in combination with an NK-activating cytokine. It is therefore reasonable to consider anti-PD-L1 therapy for the subset of AML patients with a fraction of PD-L1^{high/+} NK cells at CR, especially in the elderly where a substantial number of AML patients achieve CR but the vast majority relapse and die within 2 years [for example 58]. Finally, the data from this report can at least partly explain why some cancer patients with tumors lacking PD-L1 expression can respond favorably to anti-PD-L1 checkpoint inhibitor therapy. PD-L1⁺ NK cells may prove to be another important immune effector cell for checkpoint inhibitor-based cancer immunotherapy.

Materials and Methods

Patient Samples

Peripheral blood samples from 48 healthy donors and 79 patients newly diagnosed with AML were recruited in this study. The diagnosis and classification of AML patients were based on the revised French-American-British (FAB) classification and the 2008 World Health Organization (WHO) criteria [for example 59, 60]. Then 47 newly diagnosed patients were followed for collection of PBMCs before and after treatment. The clinic characteristics of these 47 patients are listed in Table 1. Among these 47 patients, 31 patients achieved CR, while 16 patients did not respond to the treatment. The PD-L1 expression on the NK cells before and after the treatment among these patients was compared. The patients received standard induction chemotherapy (idarubicin 10 mg/m2/day for 3 days, cytarabine 100 mg/m2/day for 7 days). Patients were evaluated for CR after 2 cycles of standard induction chemotherapy. CR was defined by the following: bone marrow blasts less than 5%, absence of blasts with Auer rods, absence of extramedullary disease, absolute neutrophil count greater than 1.0×10⁹/L, platelet count greater than 100×10⁹/L, and independence of red cell transfusions. Patients who failed to achieve these hematologic parameters after 2 cycles of standard induction chemotherapy was considered to have chemo-resistant disease.

Mice

NSG and BALB/c mice were purchased from The Jackson Laboratory.

Cell Lines

K562 and MV-4-11 were obtained from American Type Culture Collection (ATCC) within 6 months of this study. RPMI 8226, YAC-1, MOLM-13 and AML3 cells were obtained from the laboratory of M.A.C. These cells were cultured with Roswell Park Memorial Institute 1640 medium (RPMI 1640) supplied with 10% heat-inactivated fetal bovine serum (FBS, Sigma-Aldrich). These cell lines have not been authenticated but routinely checked for free of mycoplasma contamination, determined by MycoAlert™ Mycoplasma Detection Kit (Lonza). All cell lines used did not exceed 10 passages.

Cell Culture

Peripheral blood samples from healthy donors were obtained from The American Red Cross. Human PBMCs were isolated by Percoll density gradient centrifugation. Primary human NK cells were enriched from the peripheral blood of healthy donors using an NK Cell Enrichment Kit (Miltenyi Biotec, Cat #0.130-115-818). Enriched NK cells with purity about 90% were either sorted by fluorescence-activated cell sorting (FACS) to the purity around 96% and then used or used immediately for in vitro cell culture experiments. NK cells were cultured in RPMI 1640 supplemented with 20% FBS, 100 U/ml penicillin/streptomycin, and 10 ng/ml IL-2. All cell lines were maintained in RPMI 1640 medium supplemented with 10% FBS and 100 U/ml penicillin/streptomycin. For co-culture stimulation experiments, PBMCs, enriched NK cells, or FACS-sorted NK cells were co-incubated with various cell lines including K562, MOLM-13, AML3, RPMI 8226 or MV-4-11 at an effector/target (E/T) ratio of 10:1. NK cells were cultured with 10 ng/ml IL-2 in in vitro co-incubation assays unless indicated otherwise in the figures or figure legends. For transwell assay experiments, 5×10⁵ enriched human primary NK cells were seeded in the upper chamber of a transwell plate. The lower chamber of the transwell plate was seeded with 5×10⁴ K562 myeloid leukemia cells. The transwell plate with cells was incubated at 37° C. for 20 hr.

Antibody Staining and Flow Cytometry

Cells were suspended in 100 μl PBS with 2% FBS and incubated with the indicated mAb (Table 2) at room temperature for 20 mins. After washing with 2% FBS for one time and PBS for another time, cells were fixed in 1% paraformaldehyde buffer for immediate analysis by flow cytometry using a LSRII flow cytometer (BD Biosciences). Cells used for sorting were re-suspended in RPMI 1640 containing 10% FBS. For intracellular flow cytometry, cells were permeabilized and fixed using the Foxp3/Transcription Factor Fixation/Permeabilization kit (eBioscience, Cat #00-5523-00). Data were analyzed by FlowJo software.

Immunostaining Assay

Resting NK cells or NK cells stimulated with K562 cells were seeded on a glass-bottom dish and centrifuged for 10 mins. Cells were stained with 5 μg/mL mouse anti human CD56 antibody (Invitrogen, Cat. #MA1-35249) and rabbit anti-human PD-L1 antibody (Cell Signaling Technology, Cat. #13684) according to the manufacturer's instructions. Cells were then washed and stained with goat anti-rabbit IgG conjugated with Alexa Fluor 488 (Thermos Fisher, Cat. # A-11034) and goat anti-mouse IgG conjugated with Alexa Fluor 594 (Thermos Fisher, Cat. # A-11005). Cells were then stained with DAPI (Sigma, Cat. #D9542-1MG). The stained cells were examined under a LSM 880 Laser Scanning Microscope at 20× objective.

Immunoblotting Assay

Cells were pelleted and lysed in protein extraction reagent (Thermo Fisher, Cat. #78510) supplied with proteinase inhibitors. Standard procedures were used for immunoblotting. Primary antibodies used were PD-L1 (Cell Signaling Technology, Cat. #13684), GAPDH (Cell Signaling Technology, Cat. #2118), β-actin (Cell Signaling Technology, Cat. #4967), p-p38 (Cell Signaling Technology, Cat. #9211) and p-AKT (Cell Signaling Technology, Cat. #9271). Proteins were detected using goat anti-rabbit HRP-conjugated secondary antibodies (Cell Signaling Technology, Cat. #7074).

Microarray

High quality total RNA isolated from FACS-sorted PD-L1+ and PD-L1⁻ NK cells were used for microarray analysis. The integrity and quantity of the RNA were checked by Agilent Bioanalyzer and Nanodrop RNA 6000, respectively. The Clariom™ D Assay chip was used for hybridization following the manufacturer's protocol. Gene expression profile is analyzed using transcriptome analysis console (TAC) 3.0 software. Data collected from three donors were used for microarray analysis.

Real-Time PCR

RNA was isolated using RNA Isolation kit (QIAGEN, Cat. #74106) according to manufacturer's instructions, and cDNA was synthesized using cDNA Synthesis Kit (Thermo Fisher Scientific, Cat. #18080051). The data were collected using a StepOnePlus Real-Time PCR System (Thermo Fisher Scientific) using a reaction protocol of 95° C. for 1 min, followed by 40 cycles of 95° C. for 10 seconds, 60° C. for 30 seconds, and 72° C. for 30 seconds. PD-L1-F: TGGCATTTGCTGAACGCATTT (SEQ ID NO:1); PD-L1-R: TGCAGCCAGGTCTAATTGTTTT (SEQ ID NO:2).

ChIP Assay

Chromatin immunoprecipitation (ChIP) assay was performed using Pierce™ Agarose ChIP Kit from Thermo Scientific™, followed by the manufacturer's instruction. Briefly, 293T cells were transfected with the PD-L1 promoter alone or together with AKT, p38, p65 or empty vector control for 24 hour. The cells were cross-linked at 1% formaldehyde and washed once with Glycine Solution. The chromatin was collected form cell lysate and digested into 20-1000 bp segments by MNase. Digested chromatin was incubated overnight with a p65 ChIP-grade antibody (Cell Signaling Technology, Cat. #8242) or IgG control antibody (Cell Signaling Technology, Cat. #3900). The enriched chromatin was analysis by Real-time PCR (RT-PCR).

Primer-PD-L1 promoter-F:
(SEQ ID NO: 3)
TCAGTCACCTTGAAGAGGCT;
Primer-PD-L1 promoter-R:
(SEQ ID NO: 4)
TTTCACCGGGAAGAGTTTCG.

Cytotoxicity Assay

Cytotoxicity assays were performed as described previously [61]. K562 target cells were labeled with ⁵¹Cr for 1 hour at 37° C. Cells were washed and co-incubated with effector cells (PD-L1+ and PD-L1⁻ NK cells) in a 96-well V-bottom plate at various E/T ratios for 4 ourh at 37° C. Following culture, supernatants were collected in a scintillation vial for analysis. The standard formula of 100×(cpmₑₓₚₑᵣᵢₘₑₙₜₐₗ−cpmₛₚₒₙₜₐₙₑₒᵤₛ)/(cpmₘₐₓᵢₘₐₗ−cpmₛₚₒₙₜₐₙₑₒᵤₛ) was used to calculate percentages of specific lysis, which were displayed as the mean of triplicate samples.

PD-L1-Knockout Cell Line

PD-L1-knockout K562 and YAC-1 cells were generated using CRISPR/Cas9 knockout plasmids purchased from Santa Cruz and used according to manufacturer's instructions. K562 and YAC-1 cells were co-transfected with the homology-directed DNA repair (HDR) plasmid, which incorporates a puromycin resistance gene for selection of cells containing a successful Cas9-induced site-specific human/murine-PD-L1 knockout in genomic DNA. The cells were then selected with media containing 2 μg/mL puromycine. The expression of PD-L1 was examined by flow cytometry.

NSG Mouse Model

8-week old NSG mice were intravenously (i.v.) injected with PD-L1 knockout K562 myeloid leukemia cells at a concentration of 2 million cells per mouse. One day later, each mouse was i.v-injected once with 20 million human primary NK cells and intra-peritoneally (i.p.) injected with IL-2 alone, the combination of IL-12 and IL-15, or the combination or IL-12, IL-15 and IL-18 at a dose of 0.5 μg for each cytokine per mouse. Mice in the atezolizumab (AZ)-treated or control group were simultaneously i.p-injected with 200 μg AZ in 200 μL PBS or 200 μL PBS per mouse. Cytokines and AZ were injected every other day for a total of 7 times. The numbers of NK cells and tumor cells were examined at day 6 post injection.

YAC-1 Mouse Model

8-week old wildtype (WT) and PD-L1^{-/-} BALB/c mice were intra-peritoneally (i.p.)-injected with an anti-PD-L1 antibody or an IgG control antibody at a concentration of 500 μg per mouse. To deplete NK cells, mice were i.p-injected with 10 μL anti-asialo-GM1 antibody one day before inoculation of YAC-1 tumor cells. One day later, mice were intra-venously (i.v.) injected with PD-L1-knockout YAC-1 cells (PD-L1 KO YAC-1) at the dose of 1 million cells per mouse. The antibodies were administered every three days at a dose of 200 μg per mouse for four weeks. The numbers of immune cells and tumor cells were examined at day 30 post injection.

Statistical Analysis

Two independent or paired groups were compared by Student's t-test or paired t-test. Multiple groups were compared using ANOVA models or linear mixed models for repeated measures. For survival data, Kaplan-Meier method was used to estimate survival functions and logrank test was applied to group comparisons. P values were corrected for multiple comparisons by Holm's method or the Holm-Sidak method. A P value less than 0.05 was considered statistically significant (* P<0.05; **P<0.01,***, P<0.001; ****, P<0.0001). For microarray data, paired t test with variance smoothing was applied to group comparisons for each gene after log base 2 transformation and noise-level gene filtering. Both fold change and mean number of false positives (i.e. 5 out of 10,000 genes) were used to identify the top genes.

INFORMAL SEQUENCE LISTING
PD-L1-F primer
(SEQ ID NO: 1)
TGGCATTTGCTGAACGCATTT
PD-L1-R primer
(SEQ ID NO: 2)
TGCAGCCAGGTCTAATTGTTTT
Primer-PD-L1 promoter-F
(SEQ ID NO: 3)
TCAGTCACCTTGAAGAGGCT
Primer-PD-L1 promoter-R
(SEQ ID NO: 4)
TTTCACCGGGAAGAGTTTCG

### REFERENCES

1. Chen, L. and X. Han, Anti-PD-1/PD-L1 therapy of human cancer: past, present, and future. J Clin Invest, 2015. 125(9): p. 3384-91.
2. Rihawi, K., et al., Pembrolizumab in the treatment of metastatic non-small cell lung cancer: a review of current evidence. Ther Adv Respir Dis, 2017. 11(9): p. 353-373.
3. Atkins, M. B., et al., Axitinib in combination with pembrolizumab in patients with advanced renal cell cancer: a non-randomised, open-label, dose-finding, and dose-expansion phase 1b trial. Lancet Oncol, 2018. 19(3): p. 405-415.
4. Ferris, R. L., et al., Nivolumab for Recurrent Squamous-Cell Carcinoma of the Head and Neck. N Engl J Med, 2016. 375(19): p. 1856-1867.
5. Santini, F. C. and C. M. Rudin, Atezolizumab for the treatment of non-small cell lung cancer. Expert Rev Clin Pharmacol, 2017. 10(9): p. 935-945.
6. Kaufman, H. L., et al., Avelumab in patients with chemotherapy-refractory metastatic Merkel cell carcinoma: a multicentre, single-group, open-label, phase 2 trial. Lancet Oncol, 2016. 17(10): p. 1374-1385.
7. Faiena, I., et al., Durvalumab: an investigational anti-PD-L1 monoclonal antibody for the treatment of urothelial carcinoma. Drug Des Devel Ther, 2018. 12: p. 209-215.
8. Herbst, R. S., et al., Predictive correlates of response to the anti-PD-L1 antibody MPDL3280A in cancer patients. Nature, 2014. 515(7528): p. 563-7.
9. Powles, T., et al., MPDL3280A (anti-PD-L1) treatment leads to clinical activity in metastatic bladder cancer. Nature, 2014. 515(7528): p. 558-62.
10. Mandai, M., et al., Dual Faces of IFNgamma in Cancer Progression: A Role of PD-L1 Induction in the Determination of Pro- and Antitumor Immunity. Clin Cancer Res, 2016. 22(10): p. 2329-34.
11. Hartley, G. P., et al., Programmed Cell Death Ligand 1 (PD-L1) Signaling Regulates Macrophage Proliferation and Activation. Cancer Immunol Res, 2018. 6(10): p. 1260-1273.
12. Iraolagoitia, X. L., et al., NK Cells Restrain Spontaneous Antitumor CD8⁺ T Cell Priming through PD-1/PD-L1 Interactions with Dendritic Cells. J Immunol, 2016. 197(3): p. 953-61.
13. Latchman, Y. E., et al., PD-L1-deficient mice show that PD-L1 on T cells, antigen-presenting cells, and host tissues negatively regulates T cells. Proc Natl Acad Sci USA, 2004. 101(29): p. 10691-6.
14. Terme, M., et al., Cancer-induced immunosuppression: IL-18-elicited immunoablative NK cells. Cancer Res, 2012. 72(11): p. 2757-67.
15. Lanier, L. L., NK cell recognition. Annu Rev Immunol, 2005. 23: p. 225-74.
16. Bubenik, J., Tumour MHC class I downregulation and immunotherapy (Review). Oncol Rep, 2003. 10(6): p. 2005-8.
17. Caligiuri, M. A., Human natural killer cells. Blood, 2008. 112(3): p. 461-9.
18. Becknell, B. and M. A. Caligiuri, Natural killer cells in innate immunity and cancer. J Immunother, 2008. 31(8): p. 685-92.
19. Pardoll, D. M., The blockade of immune checkpoints in cancer immunotherapy. Nat Rev Cancer, 2012. 12(4): p. 252-64.
20. Alter, G., J. M. Malenfant, and M. Altfeld, CD107a as a functional marker for the identification of natural killer cell activity. J Immunol Methods, 2004. 294(1-2): p. 15-22.
21. Beider, K., et al., Involvement of CXCR4 and IL-2 in the homing and retention of human NK and NK T cells to the bone marrow and spleen of NOD/SCID mice. Blood, 2003. 102(6): p. 1951-1958.
22. Weinstock, C., et al., U.S. Food and Drug Administration Approval Summary: Atezolizumab for Metastatic Non-Small Cell Lung Cancer. Clin Cancer Res, 2017. 23(16): p. 4534-4539.
23. Berthon, C., et al., In acute myeloid leukemia, B7-H1 (PD-L1) protection of blasts from cytotoxic T cells is induced by TLR ligands and interferon-gamma and can be reversed using MEK inhibitors. Cancer Immunol Immunother, 2010. 59(12): p. 1839-49.
24. Zhou, J., et al., Liver-Resident NK Cells Control Antiviral Activity of Hepatic T Cells via the PD-1-PD-L1 Axis. Immunity, 2019. 50(2): p. 403-417 e4.
25. Blank, C., et al., PD-L1/B7H-1 inhibits the effector phase of tumor rejection by T cell receptor (TCR) transgenic CD8+ T cells. Cancer Res, 2004. 64(3): p. 1140-5.
26. Yu, J., et al., Pro- and antiinflammatory cytokine signaling: reciprocal antagonism regulates interferon-gamma production by human natural killer cells. Immunity, 2006. 24(5): p. 575-90.
27. Gordon, S. M., et al., The transcription factors T-bet and Eomes control key checkpoints of natural killer cell maturation. Immunity, 2012. 36(1): p. 55-67.
28. Luetke-Eversloh, M., et al., NK cells gain higher IFN-gamma competence during terminal differentiation. Eur J Immunol, 2014. 44(7): p. 2074-84.
29. Zhang, Z., et al., DNAM-1 controls NK cell activation via an ITT-like motif. J Exp Med, 2015. 212(12): p. 2165-82.
30. Trotta, R., et al., TGF-beta utilizes SMAD3 to inhibit CD16-mediated IFN-gamma production and antibody-dependent cellular cytotoxicity in human NK cells. J Immunol, 2008. 181(6): p. 3784-92.
31. Martini, M., et al., PI3K/AKT signaling pathway and cancer: an updated review. Ann Med, 2014. 46(6): p. 372-383.
32. Wang, Y., et al., The IL-15-AKT-XBP1s signaling pathway contributes to effector functions and survival in human NK cells. Nat Immunol, 2019. 20(1): p. 10-17.
33. Deng, Y., et al., Transcription factor Foxo1 is a negative regulator of natural killer cell maturation and function. Immunity, 2015. 42(3): p. 457-70.
34. Mancini, M. and A. Toker, NFAT proteins: emerging roles in cancer progression. Nat Rev Cancer, 2009. 9(11): p. 810-20.
35. Tak, P. P. and G. S. Firestein, NF-κB: a key role in inflammatory diseases. J Clin Invest, 2001. 107(1): p. 7-11.
36. Mavropoulos, A., et al., Stabilization of IFN-gamma mRNA by MAPK p38 in IL-12- and IL-18-stimulated human NK cells. Blood, 2005. 105(1): p. 282-8.
37. Dong, P., et al., Tumor-Intrinsic PD-L1 Signaling in Cancer Initiation, Development and Treatment: Beyond Immune Evasion. Front Oncol, 2018. 8: p. 386.
38. Kamphorst, A. O., et al., Rescue of exhausted CD8 T cells by PD-1-targeted therapies is CD28-dependent. Science, 2017. 355(6332): p. 1423-1427.
39. Simoni, Y., et al., Bystander CD8(+) T cells are abundant and phenotypically distinct in human tumour infiltrates. Nature, 2018. 557(7706): p. 575-579.
40. Zheng, C., et al., Landscape of Infiltrating T Cells in Liver Cancer Revealed by Single-Cell Sequencing. Cell, 2017. 169(7): p. 1342-1356 e16.
41. Liu, Y., et al., Increased expression of programmed cell death protein 1 on NK cells inhibits NK-cell-mediated anti-tumor function and indicates poor prognosis in digestive cancers. Oncogene, 2017. 36(44): p. 6143-6153.
42. Benson, D. M., Jr., et al., The PD-1/PD-L1 axis modulates the natural killer cell versus multiple myeloma effect: a therapeutic target for CT-011, a novel monoclonal anti-PD-1 antibody. Blood, 2010. 116(13): p. 2286-94.
43. Pesce, S., et al., Identification of a subset of human natural killer cells expressing high levels of programmed death 1: A phenotypic and functional characterization. J Allergy Clin Immunol, 2017. 139(1): p. 335-346 e3.
44. Hui, E., et al., T cell costimulatory receptor CD28 is a primary target for PD-1-mediated inhibition. Science, 2017. 355(6332): p. 1428-1433.
45. Sanmamed, M. F. and L. Chen, A Paradigm Shift in Cancer Immunotherapy: From Enhancement to Normalization. Cell, 2018. 175(2): p. 313-326.
46. Schwartzentruber, D. J., et al., gp100 peptide vaccine and interleukin-2 in patients with advanced melanoma. N Engl J Med, 2011. 364(22): p. 2119-27.
47. Ascierto, P. A., et al., Ipilimumab 10 mg/kg versus ipilimumab 3 mg/kg in patients with unresectable or metastatic melanoma: a randomised, double-blind, multicentre, phase 3 trial. Lancet Oncol, 2017. 18(5): p. 611-622.
48. Lenzi, R., et al., Phase II study of intraperitoneal recombinant interleukin-12 (rhIL-12) in patients with peritoneal carcinomatosis (residual disease <1 cm) associated with ovarian cancer or primary peritoneal carcinoma. J Transl Med, 2007. 5: p. 66.
49. Gollob, J. A., et al., Phase I trial of twice-weekly intravenous interleukin 12 in patients with metastatic renal cell cancer or malignant melanoma: ability to maintain IFN-gamma induction is associated with clinical response. Clin Cancer Res, 2000. 6(5): p. 1678-92.
50. Conlon, K. C., et al., Redistribution, hyperproliferation, activation of natural killer cells and CD8 T cells, and cytokine production during first-in-human clinical trial of recombinant human interleukin-15 in patients with cancer. J Clin Oncol, 2015. 33(1): p. 74-82.
51. Robertson, M. J., et al., A Dose-escalation Study of Recombinant Human Interleukin-18 in Combination With Ofatumumab After Autologous Peripheral Blood Stem Cell Transplantation for Lymphoma. J Immunother, 2018. 41(3): p. 151-157.
52. Tugues, S., et al., New insights into IL-12-mediated tumor suppression. Cell Death Differ, 2015. 22(2): p. 237-46.
53. Perez-Martinez, A., et al., A phase I/II trial of interleukin-15-stimulated natural killer cell infusion after haplo-identical stem cell transplantation for pediatric refractory solid tumors. Cytotherapy, 2015. 17(11): p. 1594-603.
54. Yan, Y., et al., Combined therapy with CTL cells and oncolytic adenovirus expressing IL-15-induced enhanced antitumor activity. Tumour Biol, 2015. 36(6): p. 4535-43.
55. Sim, G. C. and L. Radvanyi, The IL-2 cytokine family in cancer immunotherapy. Cytokine Growth Factor Rev, 2014. 25(4): p. 377-90.
56. Madera, S. and J. C. Sun, Cutting edge: stage-specific requirement of IL-18 for antiviral NK cell expansion. J Immunol, 2015. 194(4): p. 1408-12.
57. Chaix, J., et al., Cutting edge: Priming of NK cells by IL-18. J Immunol, 2008. 181(3): p. 1627-31.
58. Kolitz, J. E., Very late relapse of acute myeloid leukemia. Leuk Lymphoma, 2007. 48(1): p. 3-4.
59. Bennett, J. M., et al., Proposed revised criteria for the classification of acute myeloid leukemia. A report of the French-American-British Cooperative Group. Ann Intern Med, 1985. 103(4): p. 620-5.
60. Vardiman, J. W., et al., The 2008 revision of the World Health Organization (WHO) classification of myeloid neoplasms and acute leukemia: rationale and important changes. Blood, 2009. 114(5): p. 937-51.
61. Yu, J., et al., Pro- and Antiinflammatory Cytokine Signaling: Reciprocal Antagonism Regulates Interferon-gamma Production by Human Natural Killer Cells. Immunity, 2006. 24(5): p. 575-590.
62. Denman C J, Senyukov V V, Somanchi S S, Phatarpekar P V, Kopp L M, Johnson J L, et al. Membrane-bound IL-21 promotes sustained ex vivo proliferation of human natural killer cells. PLoS One 2012; 7:e30264.

### P-Embodiments

Embodiment P-1. A method of treating cancer in a subject comprising:
a. detecting an amount of PD-L1(+) natural killer (NK) cells in a biological sample from said subject; and
b. treating said subject with an anticancer therapy.
   Embodiment P-2. The method of Embodiment P-1, wherein the cancer is acute myeloid leukemia or lung cancer.
   Embodiment P-3. The method of any one of Embodiments P-1 or P-2, wherein the cancer comprises PDL1-negative tumor cells.
   Embodiment P-4. The method of any one of Embodiments P-1 or P-2, wherein the cancer comprises PDL1-positive tumor cells.
   Embodiment P-5. The method of any one of Embodiments P-1-P-4, wherein detecting comprises a method selected from flow cytometry, fluorescence-activated cell sorting, antibody cell staining, immunohistochemistry (IHC), reverse transcriptase-quantitative polymerase chain reaction (RT-qPCR), immunofluorescent assay, and a combination thereof.
   Embodiment P-6. The method of any one of Embodiments P-1-P-5, wherein the amount of PD-L1(+) NK cells is about equal to or greater than the amount of PD-L1(−) NK cells.
   Embodiment P-7. The method of Embodiment P-6, wherein more PD-L1(+) NK cells correlates with increased response to anti-cancer therapy.
   Embodiment P-8. The method of any one of Embodiments P-1-P-7, wherein the anticancer therapy is selected from chemotherapy, radiation therapy, surgery, targeted therapy, immunotherapy, and cell therapy.
   Embodiment P-9. The method of Embodiment P-8, wherein the immunotherapy comprises a checkpoint inhibitor.
   Embodiment P-10. The methods of Embodiment P-9, wherein the checkpoint inhibitor is a PD-1 inhibitor.
   Embodiment P-11. The method of Embodiment P-10, wherein the PD-1 inhibitor is pembrolizumab and nivolumab.
   Embodiment P-12. The methods of Embodiment P-9, wherein the checkpoint inhibitor is a PD-L1 inhibitor.
   Embodiment P-13. The method of Embodiment P-12, wherein the PD-L1 inhibitor is selected from atezolizumab, avelumab, and durvalumab.
   Embodiment P-14. The method of Embodiment P-13, wherein the PD-L1 inhibitor is atezolizumab.
   Embodiment P-15. The method of Embodiment P-8, wherein the cell therapy comprises PD-L1(+) NK cells.
   Embodiment P-16. The method of any one of Embodiments P-1-P-15, wherein the anticancer therapy comprises a PD-L1 inhibitor and PD-L1(+) NK cells.
   Embodiment P-17. The method of Embodiment P-16, wherein the PD-L1(+) NK cells are enriched or purified.
   Embodiment P-18. The method of any one of Embodiments P-1-P-15, wherein the anticancer therapy comprises a PD-L1 inhibitor and bulk NK cells comprising PD-L1(+) NK cells.
   Embodiment P-19. The method of any one of Embodiments P-1-P-15, wherein the anticancer therapy comprises a PD-L1 inhibitor and a NK cell activating agent.
   Embodiment P-20. The method of Embodiment P-19, wherein the NK cell-activating agent is a feeder cell.
   Embodiment P-21. The method of Embodiment P-20, wherein the feeder cell is selected from a K562 cell and a K562 cell expressing IL-15 and/or IL-21.
   Embodiment P-22. The method of Embodiment P-19 wherein the NK cell-activating agent is a cytokine.
   Embodiment P-23. The method of Embodiment P-22, wherein the cytokine is selected from IL-2, IL-12, IL-15, IL-18, and a combination thereof.
   Embodiment P-24. A method of treating cancer in a patient comprising:
a. isolating natural killer (NK) cells from a subject thereby producing a population of isolated NK cells;
b. deriving a population of PD-L1(+) NK cell from said population of isolated NK cells; and
c. administering the population of PD-L1(+) NK cells into said patient.
   Embodiment P-25. The method of Embodiment P-24, wherein the cancer is acute myeloid leukemia or lung cancer.
   Embodiment P-26. The method of any one of Embodiments P-24 or P-25, wherein the cancer comprises PD-L1(−) tumor cells.
   Embodiment P-27. The method of any one of Embodiment P-24 or P-25, wherein the cancer comprises PD-L1(+) tumor cells.
   Embodiment P-28. The method of any one of Embodiments P-24-P-27, wherein said patient is selected from a newly diagnosed cancer patient, a cancer patient relapsed from a treatment, or a cancer patient that has undergone hematopoietic stem cell transplantation.
   Embodiment P-29. The method of any one of Embodiments P-24-P-28, wherein said patient has PD-L1 (+) NK cells, has no PD-L1 (+) NK cells, has an NK cell deficiency, or has NK cell suppression.
   Embodiment P-30. The method of any one of Embodiments P-24-P-29, wherein isolating comprises fluorescence-activated cell sorting, magnetic bead separation, and/or column purification.
   Embodiment P-31. The method of any one of Embodiments P-24-P-30, wherein the subject is selected from an autologous cancer patient, a healthy donor, a matched heterologous hematopoietic stem cell donor, and a partially matched heterologous hematopoietic stem cell donor.
   Embodiment P-32. The method of any one of Embodiment P-24-P-31, wherein deriving comprises expanding PD-L1(+) NK cells by exposing the isolated NK cells to a feeder cell thereby producing a population of PD-L1(+) NK cell.
   Embodiment P-33. The method of Embodiment P-32, wherein the feeder cell is selected from a K562 cell and a K562 cell expressing IL-15 and/or IL-21.
   Embodiment P-34. The method of any one of Embodiment P-24-P-31, wherein deriving comprises fluorescence-activated cell sorting, magnetic bead separation, and/or column purification thereby producing a population of PD-L1(+) NK cell.
   Embodiment P-35. The method of any one of Embodiment P-24-P-31, wherein deriving comprises exposing the isolated NK cells to an NK activating agent to induce PD-L1 expression thereby producing a population of PD-L1(+) NK cell.
   Embodiment P-36. The method of Embodiment P-35, wherein the population of PD-L1(+) NK cell is expanded prior to administering into the patient.
   Embodiment P-37. The method of any one of Embodiments P-35 or P-36, wherein the NK cell-activating agent is a cytokine selected from IL-2, IL-12, IL-15, IL-18, and a combination thereof.
   Embodiment P-38. The method of any one of Embodiment P-35 or P-36, wherein the NK cell-activating agent is a feeder cell.
   Embodiment P-39. The method of any one of Embodiments P-24-P-31, wherein deriving comprises genetically engineering PD-L1 expression in the population of isolated NK cells thereby producing a population of PD-L1(+) NK cell.
   Embodiment P-40. The method of any one of Embodiments P-24-P-39, further comprising administering an anticancer therapy selected from chemotherapy, radiation therapy, surgery, targeted therapy, immunotherapy, cell therapy, and a combination thereof.
   Embodiment P-41. The method of Embodiment P-40, wherein the immunotherapy comprises a checkpoint inhibitor.
   Embodiment P-42. The methods of Embodiment P-41, wherein the checkpoint inhibitor is a PD-1 inhibitor.
   Embodiment P-43. The method of Embodiment P-42, wherein the PD-1 inhibitor is pembrolizumab and nivolumab.
   Embodiment P-44. The methods of Embodiment P-40, wherein the checkpoint inhibitor is a PD-L1 inhibitor.
   Embodiment P-45. The method of Embodiment P-44, wherein the PD-L1 inhibitor is selected from atezolizumab, avelumab, and durvalumab.
   Embodiment P-46. The method of claim 45, wherein the PD-L1 inhibitor is atezolizumab,
   Embodiment P-47. The method of any one of Embodiments P-24-P-40, wherein the anticancer therapy comprises an NK cell-activating agent.
   Embodiment P-48. The method of Embodiment P-47, wherein the NK cell-activating agent is a cytokine.
   Embodiment P-49. The method of Embodiment P-48, wherein the cytokine is selected from IL-2, IL-12, IL-15, IL-18, and a combination thereof.
   Embodiment P-50. A method of treating cancer in a subject comprising administering an NK cell activating agent and an immunotherapeutic to said subject.
   Embodiment P-51. The method of Embodiment P-50, wherein the NK cell activating agent is a feeder cell.
   Embodiment P-52. The method of Embodiment P-51, wherein the NK cell activating agent is a cytokine.
   Embodiment P-53. The method of Embodiment P-52, wherein the cytokine is selected from IL-2, IL-12, IL-15, IL-18, and a combination thereof.
   Embodiment P-54. The method of any one of Embodiments P-50-P-53, wherein the immunotherapeutic is a check point inhibitor.
   Embodiment P-55. The method of Embodiment P-54, wherein the checkpoint inhibitor is a PD-1 inhibitor.
   Embodiment P-56. The method of Embodiment P-55, wherein the PD-1 inhibitor is pembrolizumab and nivolumab.
   Embodiment P-57. The methods of Embodiment P-54, wherein the checkpoint inhibitor is a PD-L1 inhibitor.
   Embodiment P-58. The method of Embodiment P-57, wherein the PD-L1 inhibitor is selected from atezolizumab, avelumab, and durvalumab.
   Embodiment P-59. The method of Embodiment P-58, wherein the PD-L1 inhibitor is atezolizumab.

1. A method of treating cancer in a subject comprising:
a. detecting an amount of PD-L1(+) natural killer (NK) cells in a biological sample from said subject; and
b. treating said subject with an anticancer therapy.

2.-5. (canceled)

6. The method of claim 1, wherein the amount of PD-L1(+) NK cells is about equal to or greater than the amount of PD-L1(−) NK cells.

7. The method of claim 6, wherein more PD-L1(+) NK cells correlates with increased response to anti-cancer therapy.

8-14. (canceled)

15. The method of claim 1, wherein the anticancer therapy comprises PD-L1(+) NK cells.

16. The method of claim 15, wherein the anticancer therapy further comprises a PD-L1 inhibitor.

17.-18. (canceled)

19. The method of claim 1, wherein the anticancer therapy comprises a PD-L1 inhibitor and a NK cell activating agent.

20.-23. (canceled)

24. A method of treating cancer in a patient comprising:
a. isolating natural killer (NK) cells from a subject thereby producing a population of isolated NK cells;
b. deriving a population of PD-L1(+) NK cell from said population of isolated NK cells; and
c. administering the population of PD-L1(+) NK cells into said patient.

25. The method of claim 24, wherein the cancer is acute myeloid leukemia or lung cancer, or wherein the cancer comprises PD-L1(−) tumor cells, or wherein the cancer comprises PD-L1(+) tumor cells.

26.-27. (canceled)

28. The method of claim 24, wherein said patient is selected from a newly diagnosed cancer patient, a cancer patient relapsed from a treatment, or a cancer patient that has undergone hematopoietic stem cell transplantation, or wherein said patient has PD-L1 (+) NK cells, has no PD-L1 (+) NK cells, has an NK cell deficiency, or has NK cell suppression, or both.

29. (canceled)

30. The method of claim 24, wherein isolating comprises fluorescence-activated cell sorting, magnetic bead separation, or column purification.

31. The method of claim 24, wherein the subject is selected from an autologous cancer patient, a healthy donor, a matched heterologous hematopoietic stem cell donor, and a partially matched heterologous hematopoietic stem cell donor.

32. The method of claim 24, wherein deriving comprises expanding PD-L1(+) NK cells by exposing the isolated NK cells to a feeder cell optionally selected from a K562 cell and a K562 cell expressing IL-15 or IL-21 or both thereby producing a population of PD-L1(+) NK cell, or wherein deriving comprises fluorescence-activated cell sorting, magnetic bead separation, or column purification thereby producing a population of PD-L1(+) NK cell, or wherein deriving comprises exposing the isolated NK cells to an NK activating agent to induce PD-L1 expression thereby producing a population of PD-L1(+) NK cell, or wherein deriving comprises genetically engineering PD-L1 expression in the population of isolated NK cells thereby producing a population of PD-L1(+) NK cell.

33.-35. (canceled)

36. The method of claim 32, wherein the population of PD-L1(+) NK cell is expanded prior to administering into the patient.

37. The method of claim 32, wherein the NK cell-activating agent is a cytokine selected from IL-2, IL-12, IL-15, IL-18, and a combination thereof, or wherein the NK cell-activating agent is a feeder cell.

38.-39. (canceled)

40. The method of claim 24, further comprising administering an anticancer therapy selected from chemotherapy, radiation therapy, surgery, targeted therapy, immunotherapy, cell therapy, and a combination thereof.

41. The method of claim 40, wherein the immunotherapy comprises a checkpoint inhibitor, optionally a PD-1 inhibitor or a PD-L1 inhibitor.

42.-44. (canceled)

45. The method of claim 41, wherein the PD-1 inhibitor is pembrolizumab or nivolumab, and wherein the PD-L1 inhibitor is selected from atezolizumab, avelumab, and durvalumab.

46. (canceled)

47. The method of claim 24, wherein the anticancer therapy comprises an NK cell-activating agent.

48. The method of claim 47, wherein the NK cell-activating agent is a cytokine, optionally selected from IL-2, IL-12, IL-15, IL-18, or a combination thereof.

49. (canceled)

50. A method of treating cancer in a subject comprising administering an NK cell activating agent and an immunotherapeutic to said subject.

51.-59. (canceled)
